(19) United States
(12) Patent Application Publication
(10) Pub. No.: US 2025/0222143 A1
(43) Pub. Date: Jul. 10, 2025

(54) TARGETED RADIOTHERANOSTICS BASED ON POLYAZAMACROCYCLIC, MIXED-DONOR SCAFFOLDS LINKED TO A TARGETING VECTOR

(71) Applicant: The Research Foundation for the State University of New York, Albany, NY (US)

(72) Inventors: Eszter BOROS, Stony Brook, NY (US); Brett VAUGHN, Stony Brook, NY (US); Jennifer WHETTER, Stony Brook, NY (US)

(73) Assignee: The Research Foundation for the State University of New York, Albany, NY (US)

(21) Appl. No.: 18/703,086

(22) PCT Filed: Oct. 19, 2022
(86) PCT No.: PCT/US2022/078389

§ 371 Date: Apr. 19, 2024

Related U.S. Application Data
(60) Provisional application No. 63/257,821, filed on Oct. 20, 2021.

(51) Int. Cl. A61K 51/04

(52) U.S. Cl. CPC A61K 51/0402; A61K 51/0406; A61K 51/0497; A61K 2123/00

(57) ABSTRACT

The present invention provides a compound having the structure:

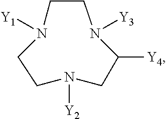

and methods of using the compound in targeted PET and SPECT imaging.

This application claims the benefit of U.S. Provisional Application No. 63/257,821, filed Oct. 20, 2021, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Picaga is a picolinic acid-functionalized triaza-cyclononane heptadentate chelator (FIG. 1A). The 7-coordinate bifunctional chelator provides a kinetically inert coordination environment for targeted in vivo applications with various radioisotopes such as scandium and lutetium. Picaga has been successfully appended to a targeting vector to incorporate 2-[3-(1,3-dicarboxypropyl)ureido]pentanedioic acid (DUPA) targeting the prostate specific membrane antigen (PSMA), commonly overexpressed in metastasizing prostate cancers (Ghosh, A. & Heston, W. D. 2004). The resulting conjugate picaga-DUPA can be radiolabeled with scandium radioisotopes at room temperature, remains inert in vitro and in vivo and produces excellent target-to-background uptake as evidenced by PET imaging and biodistribution analysis with the conjugate ⁴⁴Sc(picaga-DUPA) (Vaughn, B. A. et al. 2020). Furthermore, picaga may be chelated with either ⁴⁷Sc or ¹⁷⁷Lu to produce ⁴⁷Sc(picaga)-DUPA or ¹⁷⁷Lu (picaga)-DUPA, which demonstrated tumor-growth attenuating effects in mice bearing xenograft PSMA+PiP tumors at a dose significantly below that expected to exhibit radiotoxicities for such agents (Vaughn, B. A. et al. 2021).

⁴⁴Scandium is an ideal short-lived radioisotope with a half-life well matched to the typical pharmacokinetics of small molecules, peptides and small biologics and with ideal emission properties (t_{1/2}=3.97 h, Eₘₑₐₙ β⁺=632 keV) for PET imaging. The isotope ⁴⁷Sc, a low-energy β⁻ emitter (t₁/2=80.4 h, Eₘₑₐₙ β⁻=162 keV) is an isotope with identical chemical properties to ⁴⁴Sc and highly suited for radiotherapeutic applications (Schmitt, M. et al. 2014). ¹⁷⁷Lu is also ideally suited for a therapeutic radiometal. Its half-life (6.65 days) and β-emission energy (Avg Eβ⁻=134 keV, tissue penetration 1.5 mm) make it an attractive candidate for use in targeted radiotherapy (Price, E. W. & Orvig, C. 2014; Price, E. W. et al. 2013).

Although picaga provides a suitable approach to room temperature radiolabeling with Sc isotopes for preclinical studies, improvement of apparent molar activities (AMA) for clinical scale production of radiopharmaceuticals is desirable. Additionally, attempts to radiolabel picaga with ¹⁷⁷Lu at room temperature have been unsuccessful. Towards this end, described herein is the preparation of novel Picaga analogs with the purpose of enabling improved chelation methods. These analogs along with the chelation methods can be used to prepare conjugates with improved properties with broad application for use in imaging and therapy.

Fluorine-18 remains the most widely clinically utilized radionuclide globally and plays a pivotal role in diagnostic cancer imaging with positron emission tomography (PET). The emergence of therapeutic isotopes for the management of disease has produced a pronounced interest in matched, theranostic isotope pairs that can be employed in tandem for the diagnosis and stratification of patients for subsequent radiotherapy. F-18 however does not have a suitable therapeutic isotopologue, thus F-18 PET probes represent suboptimal diagnostic partners to chemically dissimilar, frequently radiometal-based endoradiotherapies. Here, the formation of Sc-¹⁸F ternary complexes was demonstrated to be feasible under mild, aqueous conditions, producing chemically robust radiopharmaceuticals in high radiochemical yield and specific activity. A corresponding in vivo imaging and biodistribution study with a cancer-targeting Sc-¹⁸F tracer indicates excellent in vivo stability and produces exquisite PET image quality, rendering the ¹⁸F/⁴⁷Sc isotope pair an unusual, yet chemically matched theranostic pair with excellent potential for clinical translation.

### SUMMARY OF THE INVENTION

The present invention provides a compound having the structure:

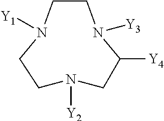

wherein
Y₁ and Y₂ are each, independently, —H, alkylheteroaryl, alkyl-CO₂H, alkylaryl-CO₂H,
   alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂, or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃; and
Y₃ and Y₄ are each, independently, —H, Z₁-L(A), Z₁-L(A)(B), L(A), L(A)(B),

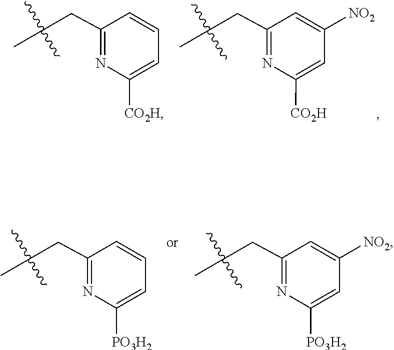

wherein
Z₁ is

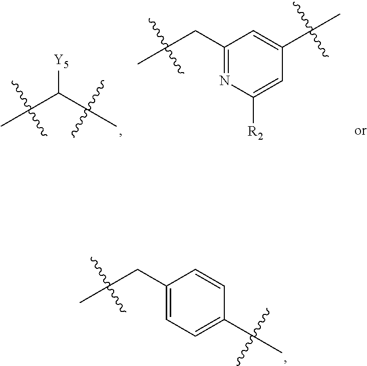

Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not

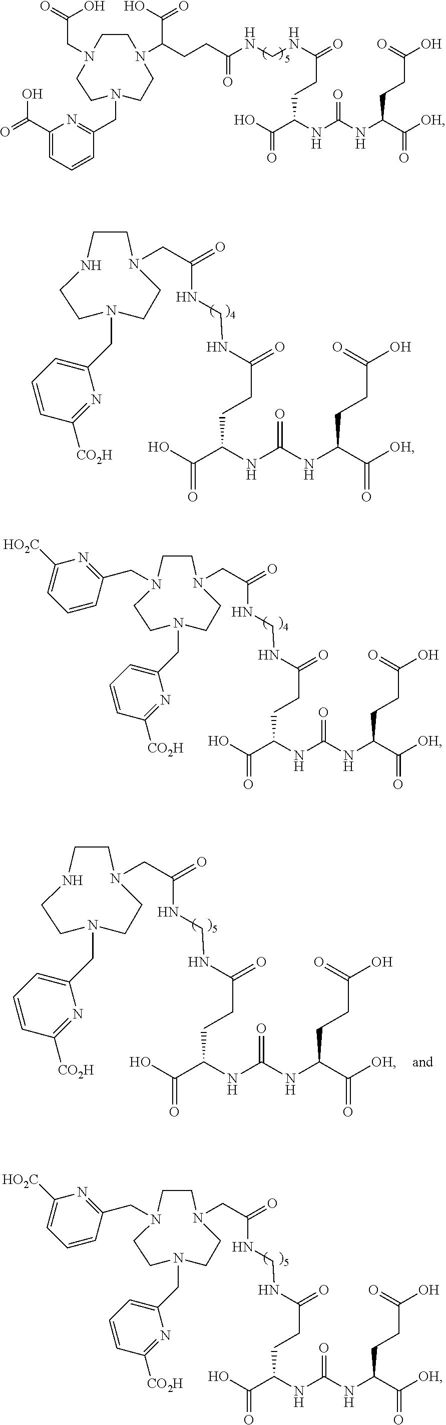

or a pharmaceutically acceptable salt of the compound.

The present invention provides a process for producing a metal complex having the structure:

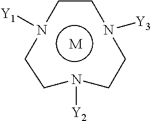

wherein
M is ⁴⁴Sc-¹⁸F, ⁴⁷Sc-¹⁸F, ¹³²La-¹⁸F, ¹³⁵La-¹⁸F or ¹⁷⁷Lu-¹⁸F,
   comprising
   (a) contacting the compound having the structure:

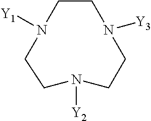

with a preformed M complex in a first suitable solvent to produce a metal complex having the structure:

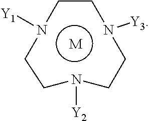

The present invention provides a method of detecting cancer cells in a subject comprising administering an effective amount of a metal complex or a composition, and imaging the subject with a molecular imaging device to detect the metal complex or composition in the subject, wherein the cancer cells are prostate cancer cells, wherein the cancer cells have elevated levels of prostate-specific membrane antigen (PSMA), wherein the metal complex or composition comprising a compound having the structure:

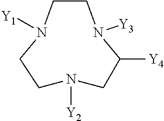

wherein:
Y₁ and Y₂ are each, independently, —H, alkylheteroaryl, alkyl-CO₂H, alkylaryl-CO₂H, alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂, or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃; and
Y₃ and Y₄ are each, independently, —H, Z₁-L(A), Z₁-L(A)(B), L(A), L(A)(B),

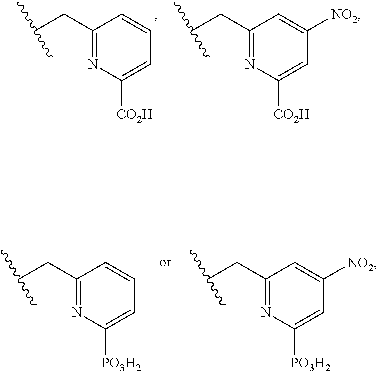

wherein
Z₁ is

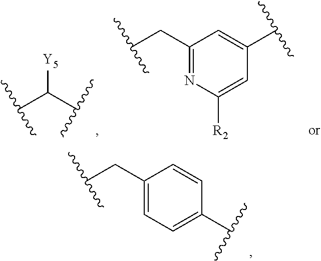

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not

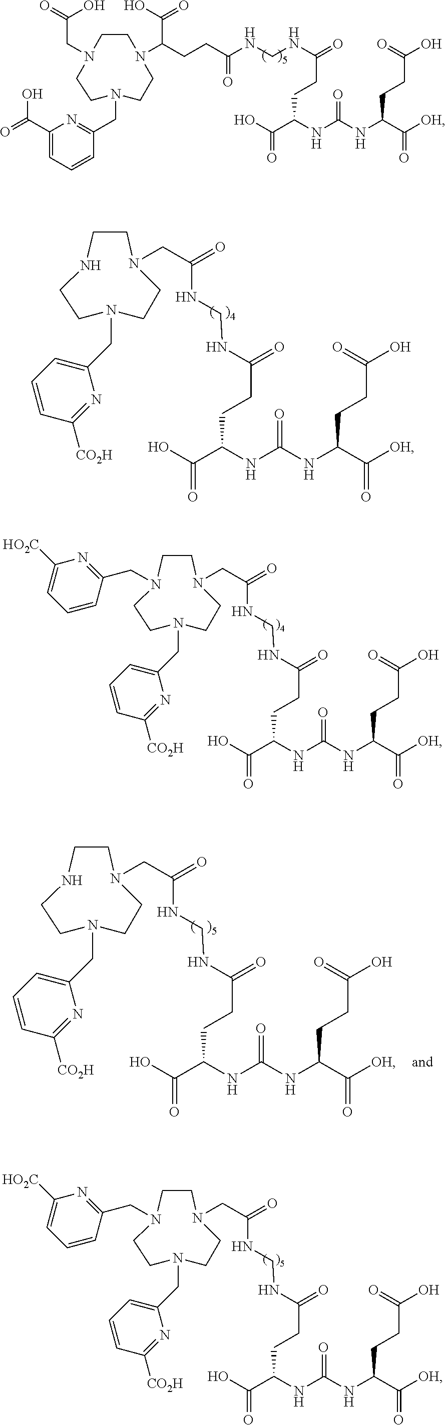

or a pharmaceutically acceptable salt of the compound;
wherein the metal or metal-ion in the metal complex is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb), or Scandium-Fluorine-18 (^{nat}Sc-¹⁸F), Lanthanum-Fluorine-18 (^{nat}La-¹⁸F), or Lutetium-Fluorine-18 (^{nat}Lu-¹⁸F).

The present invention provides a method of imaging prostate cancer cells in a subject comprising:
1) administering to the subject an effective amount of a metal complex or a pharmaceutically acceptable salt thereof, or a composition or a pharmaceutically acceptable salt thereof, wherein the compound specifically accumulates at prostate cancer cells in the subject;
2) detecting in the subject the location of the metal complex or the composition; and
3) obtaining an image of the cancer cells in the subject based on the location of the metal complex or the composition in the subject,
   wherein the metal complex or composition comprising a compound having the structure:

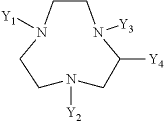

wherein
Y₁ and Y₂ are each, independently, —H, alkylheteroaryl, alkyl-CO₂H, alkylaryl-CO₂H,
   alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂, or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃; and
Y₃ and Y₄ are each, independently, —H, Z₁-L(A), Z₁-L(A)(B), L(A), L(A)(B),

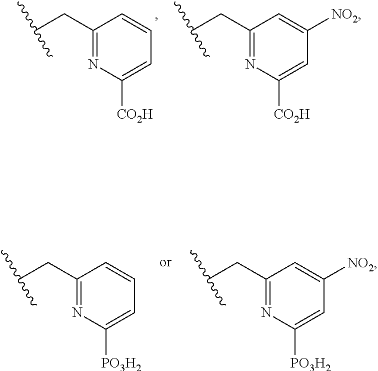

wherein
Z₁ is

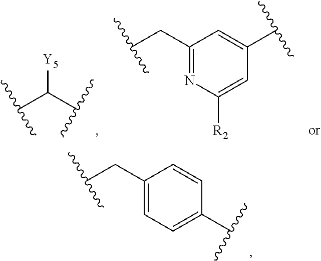

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not

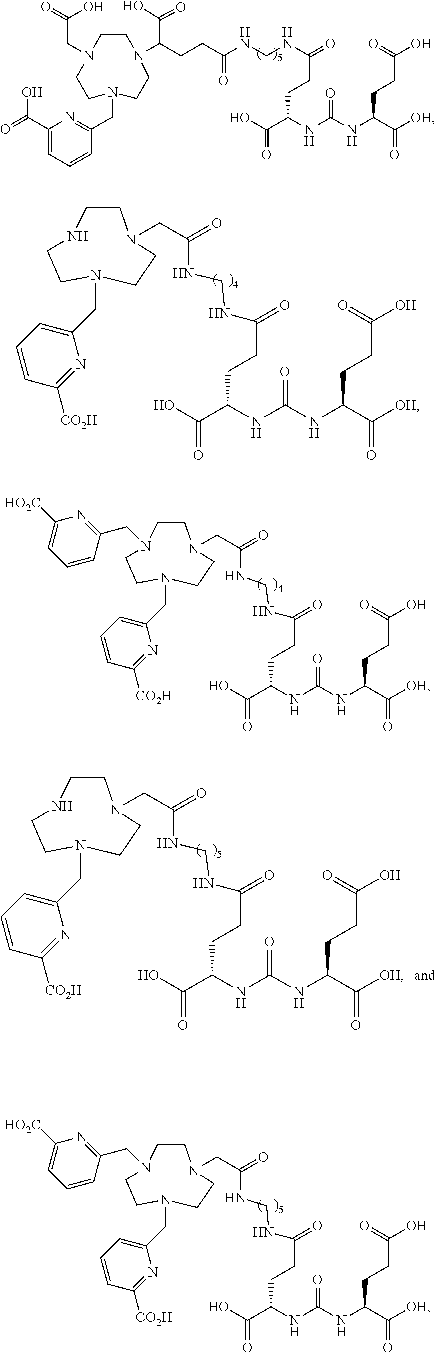

or a pharmaceutically acceptable salt of the compound;
wherein the metal or metal-ion in the metal complex is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb), or Scandium-Fluorine-18 (^{nat}Sc-¹⁸F), Lanthanum-Fluorine-18 (^{nat}La-¹⁸F), or Lutetium-Fluorine-18 (^{nat}Lu-¹⁸F).

The present invention provides a method of detecting the presence of prostate cancer cells in a subject which comprises determining if an amount of a metal complex or a pharmaceutically acceptable salt thereof, or a composition or a pharmaceutically acceptable salt thereof, is present in the subject at a period of time after administration of the metal complex or composition to the subject, thereby detecting the presence of the prostate cancer cells based on the amount of the metal complex or composition determined to be present in the subject,

wherein the metal complex or composition comprising a compound having the structure:

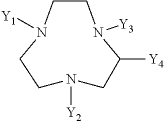

wherein
Y₁ and Y₂ are each, independently, —H, alkylheteroaryl, alkyl-CO₂H, alkylaryl-CO₂H,
   alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂, or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃; and
Y₃ and Y₄ are each, independently, —H, Z₁-L(A), Z₁-L(A)(B), L(A), L(A)(B),

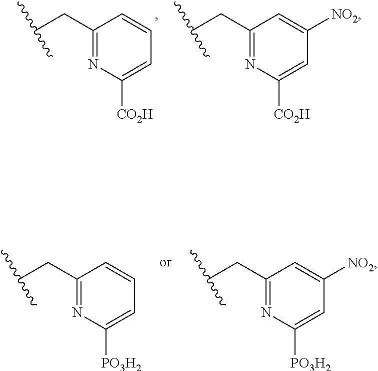

wherein
Z₁ is

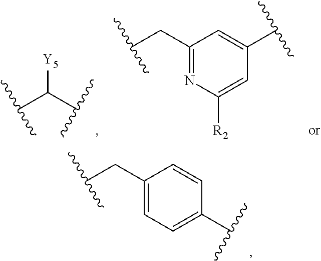

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not

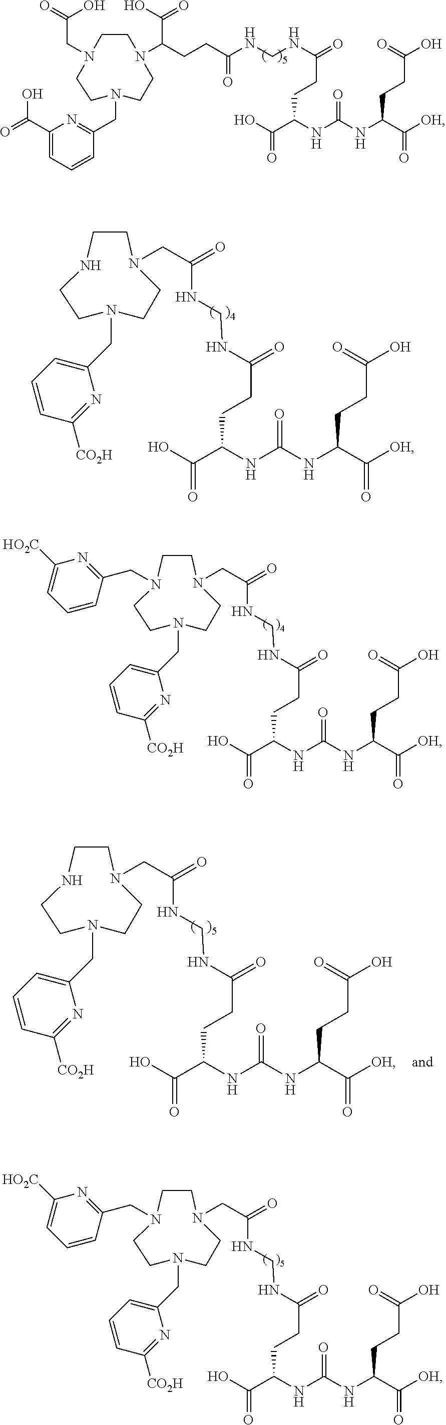

or a pharmaceutically acceptable salt of the compound;

wherein the metal or metal-ion in the metal complex is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb), or Scandium-Fluorine-18 (^{nat}Sc-¹⁸F), Lanthanum-Fluorine-18 (^{nat}La-¹⁸F), or Lutetium-Fluorine-18 (^{nat}Lu-¹⁸F).

The present invention provides a method of reducing the size of a prostate tumor or of inhibiting proliferation of prostate cancer cells comprising contacting the tumor or cancer cells with the metal complex or a pharmaceutically acceptable salt thereof, or a composition or a pharmaceutically acceptable salt thereof, so as to thereby reducing the size of the tumor or inhibit proliferation of the cancer cells, wherein the metal complex or the composition comprising a compound having the structure:

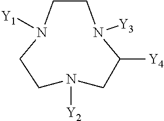

wherein
Y₁ and Y₂ are each, independently, —H, alkylheteroaryl, alkyl-CO₂H, alkylaryl-CO₂H,
   alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂, or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃; and
Y₃ and Y₄ are each, independently, —H, Z₁-L(A), Z₁-L(A)(B), L(A), L(A)(B),

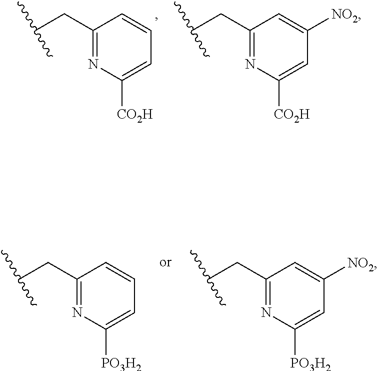

wherein
Z₁ is

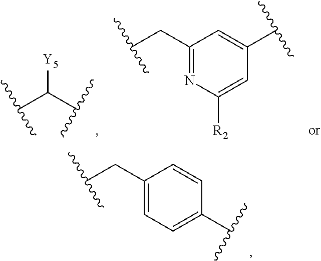

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not

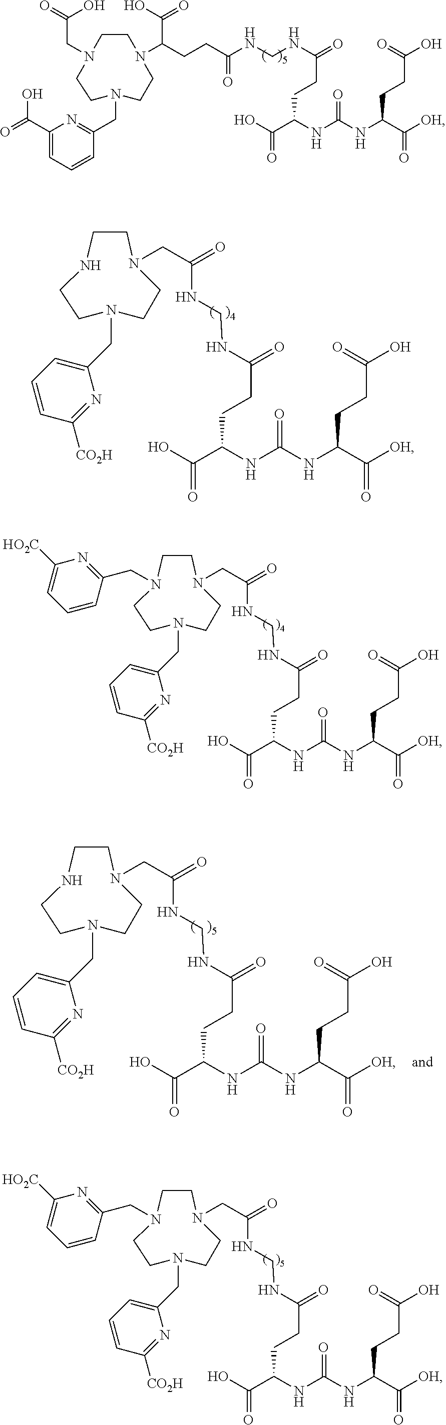

or a pharmaceutically acceptable salt of the compound;
wherein the metal or metal-ion in the metal complex is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb), or Scandium-Fluorine-18 (^{nat}Sc-¹⁸F), Lanthanum-Fluorine-18 (^{nat}La-¹⁸F), or Lutetium-Fluorine-18 (^{nat}Lu-¹⁸F).

### BRIEF DESCRIPTION OF THE FIGURES

FIG. 1: (A). Structures of Picaga (L1) and representative second-generation derivatives L2, L3 and L4. (B). Structures of additional second-generation derivatives L2, L3 and L4.

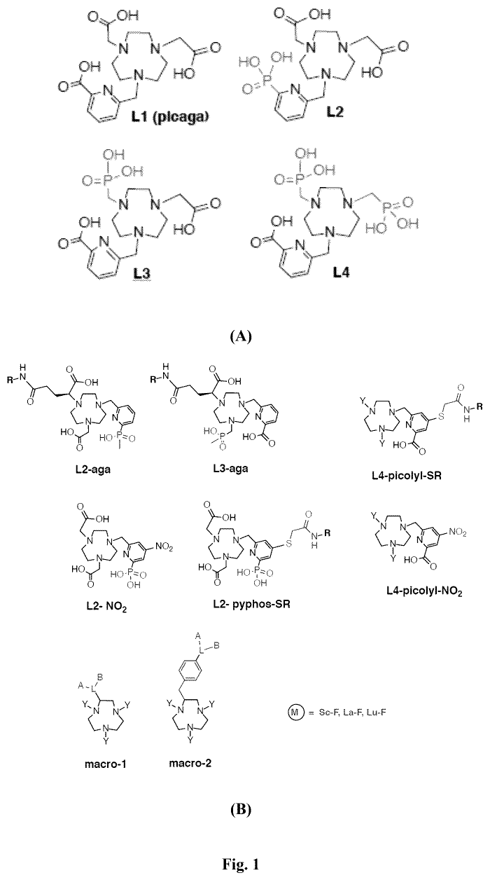

FIG. 2: Chemical structure of picaga (L1) and m-phospaten (L2) and apparent molar activity of Sc triaza-macrocycle based chelators. Respective ⁴⁴Sc radiolabeling yields in dependence of quantity of ligand employed is shown for both ⁴⁴Sc(picaga) and ⁴⁴Sc (m-phospatcn).

FIG. 3: (A). Chemical structure of first protonated complex species [Lu(HL2)]. (B). Concentration versus radiochemical yield plot of Lu-177 radiolabeling experiments with L2 demonstrates rapid and efficient radiolabeling with L2 at room temperature. (C) pH dependent speciation diagram of [Lu(L2)]-species as determined by spectrophotometric titration shows the formation of the first complex species at pH 0.9—a marked shift from 2.4 for the Lu(L1) complex.

FIG. 4: Identification of mpaten as a lead ligand for formation of complex with Sc-¹⁸F. [Sc¹⁸F(mpatcn)]⁻ is Formed by reaction of mpaten (800 nmol) with pre-formed Sc-18F complex (˜1 mCi 18F, 0.2 eq. ScCl₃) at 100° C., pH 4.

FIG. 5: (A). Optimized conditions for the preparation of [Sc¹⁸F(mpatcn)]⁻. (B). Dependence of complex formation on ligand concentration.

FIG. 6: Complexation of picaga-DUPA conjugate with preformed Sc-18F complex.

FIG. 7: Formulation stability of [Sc-¹⁸F(picaga-DUPA)]⁻ in (A) DPBS and (B) saline.

FIG. 8: (A). Biodistribution of [Sc-¹⁸F(picaga-DUPA)]⁻ in a mouse model of prostate cancer. (B). Comparison between ⁴⁴Sc-(picaga)-DUPA and clinically validated PSMA SPECT probe.

FIG. 9: PSMA-targeting tracers of A) M(PSMA-617), M³⁺=Ga³⁺/Sc³⁺/Lu³⁺/Y³⁺; B) M(picaga)-DUPA, M³⁺=Sc³⁺/Lu³⁺; C) M(PSMA-Alb-53/56), M³⁺=Lu³⁺; D) M(picaga-HSA), M³⁺=Lu³⁺; and E) M(EB-PSMA-617), M³⁺=Y³⁺/Lu³⁺.

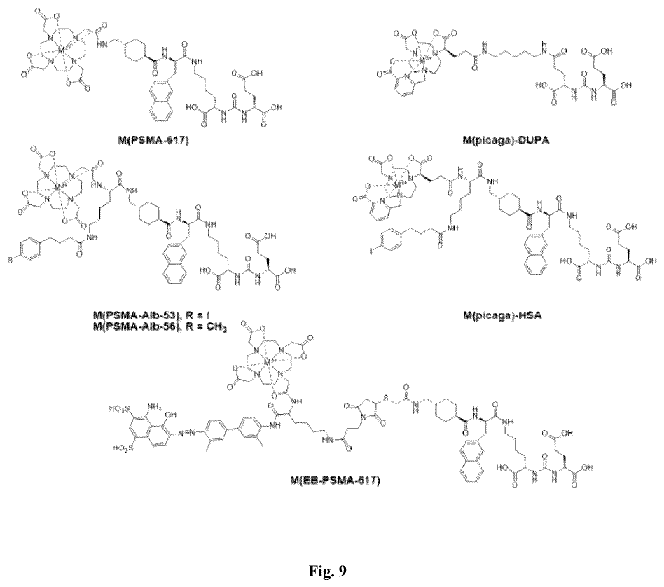

FIG. 10: Displacement assay curves obtained with concentration dependent challenge of MIP-1427 with Lu-picaga-HSA and DCFPyL.

FIG. 11: ICP-OES quantitation of Lu in filtrate of Lu-(picaga)-HSA, Lu-PSMA-617, Lu-mpaten in PBS pH 7.4 and 4.5% HSA in PBS pH 7.4.

FIG. 12: Radio-HPLC chromatograms of ¹⁷⁷Lu-PSMA-617 and ¹⁷⁷Lu-(picaga)-HSA.

FIG. 13: Cell binding and internalization of ¹⁷⁷Lu-(picaga)-HSA and ¹⁷⁷Lu-PSMA-617 in PSMA+PC-3 PIP tumor-bearing mice.

FIG. 14: (A). Comparison of biodistribution between ¹⁷⁷Lu and ⁴⁷Sc. (B). Comparison of radiotherapy efficacy between ¹⁷⁷Lu and ⁴⁷Sc. (C). A Biodistribution of ¹⁷⁷Lu-(picaga)-HSA and ¹⁷⁷Lu-PSMA-617 for 2, 24, and 72 hours post injection (n=4).

FIG. 15: SPECT images as maximum intensity projections (MIPs) of PSMA+PC3 PIP tumor bearing mice at 4 h (left), 48 h (middle), and 96 h (right) post-injection. Arrow indicates PSMA+tumor.

FIG. 16: Whole-body activity clearance from therapy cohort mice treated with ¹⁷⁷Lu-(picaga)-HSA or ¹⁷⁷Lu-PSMA-617.

FIG. 17: (A) Mean tumor growth relative to the tumor volume at Day 0 (set to 1). Therapy study performed with ¹⁷⁷Lu-(picaga)-HSA, ¹⁷⁷Lu-PSMA-617 and saline control in PC-3 PIP tumor-bearing mice. (B) Mean body weight relative to the body weight at Day 0 (set to 1). Curves are shown to the first animal euthanized in each cohort. (C) Survival curve.

FIG. 18: Relative tumor volume plot (A) and relative body weight plot (B) of mice treated with ¹⁷⁷Lu-picaga-DUPA (n=4) and saline (n=6).

FIG. 19: Kidney samples from surviving mice treated with ¹⁷⁷Lu-(picaga)-HSA (A-D), naïve mice (E-F), and ¹⁷⁷Lu-PSMA-617 (G).

FIG. 20: UV-vis titration to endpoint to determine ligand concentrations of PSMA-617 andpicaga-HSA.

FIG. 21: Relative tumor volume plots (A) and relative body weight plots (B) of mice treated with ¹⁷⁷Lu-(picaga)-HSA, ¹⁷⁷Lu-PSMA-617, and saline (n=6).

FIG. 22: HRMS of mpaten. HRMS calc. for C₁₇H₂₅N₄O₆: 381.1769. Found: 381.1765 [M+H]⁺.

FIG. 23: HRMS of Sc(mpatcn). HRMS calc. for C₁₇H₂₂N₄O₆Sc: 423.1093. Found: 423.1093 [M+H]⁺.

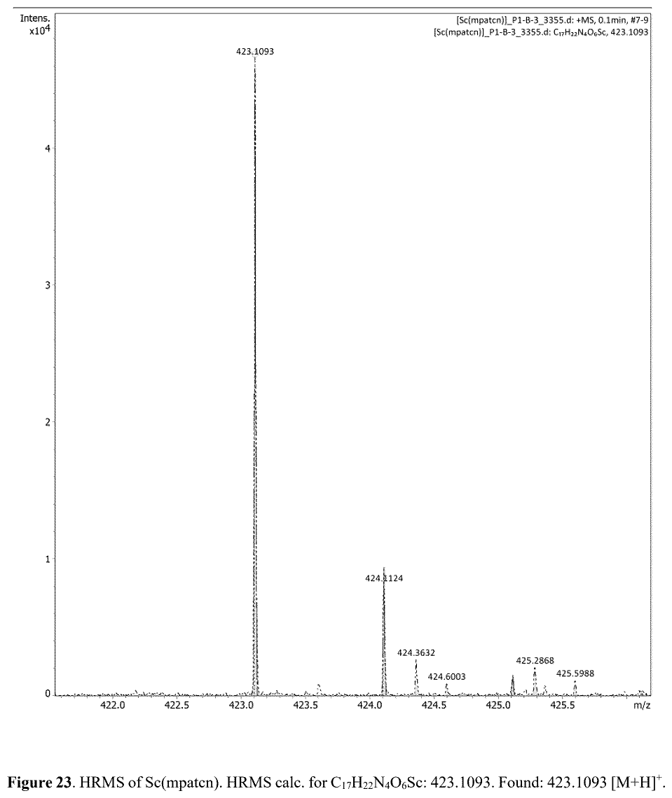

FIG. 24: HRMS of [ScF(mpatcn)]⁻. HRMS calc. for C₁₇H₂₃FN₄O₆Sc: 443.1155. Found: 443.1152. [M+2H]⁺. HRMS calc. for C₁₇H₂₁FN₄O₆Sc: 441.1010. Found: 441.1003 [M]⁻ (negative mode, not shown).

FIG. 25: (A). LCMS UV chromatogram of mpatcn. Retention time (t_{R})=1.760 min (Method A). (B). LCMS UV chromatogram of [Sc(mpatcn)]⁻. Retention time (t_{R})=1.392 min (Method A). (C). LCMS UV chromatogram of [ScF(mpatcn)]⁻. Retention time (t_{R})=1.516 min (Method B, above). Retention time (t_{R})=1.931 min (Method A, not shown).

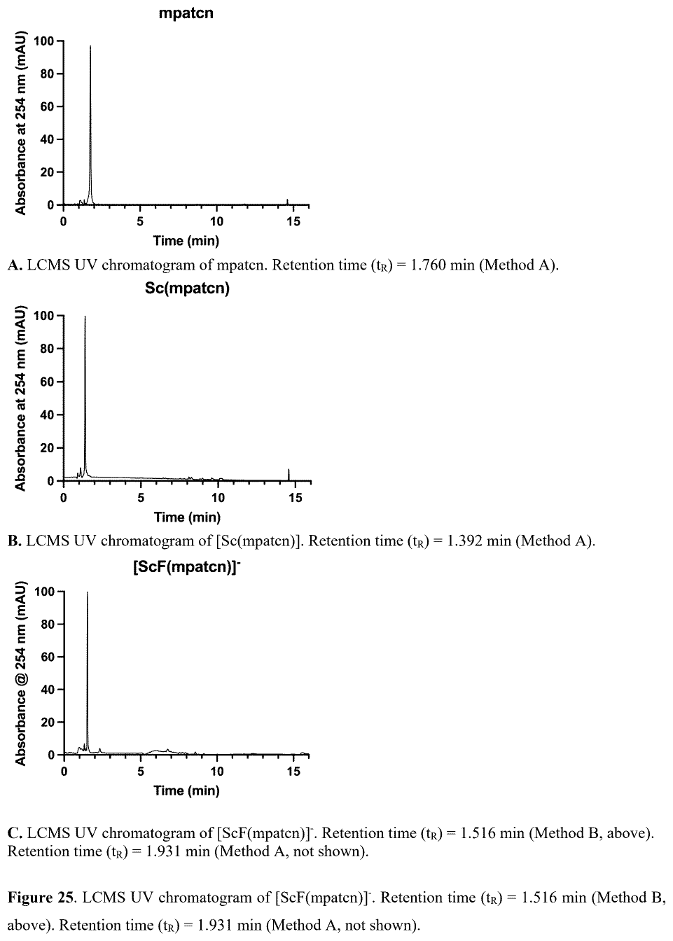

FIG. 26: ¹H NMR of mpatcn (bottom) and [ScF(mpatcn)]⁻ (top). 400 MHZ, D₂O.

FIG. 27: A. 19F NMR of NH₄F (bottom) and [ScF(mpatcn)]⁻ (top) aligned to the TFA (8-75.5) signal. 400 MHz, D₂O. NH₄F: δ −118.12. [ScF(mpatcn)]⁻: δ −15.5, broad. B. ⁴⁵Sc NMR of pH 1.5 ScCl₃ (bottom) and [ScF(mpatcn)]⁻ (top). 400 MHZ, D₂O. ScCl₃: δ −0.45. [ScF(mpatcn)]⁻: δ 61.5. [Sc(mpatcn)]: δ 80.1 (from previous work,¹ not shown).

FIG. 28: (A). Spectrophotometric titration of H₃mpaten with Cu²⁺. UV-Vis absorbance spectra of H₃mpatcn upon Cu²⁺ addition (left) and UV-vis titration to endpoint to determine ligand concentration (right). Analysis reveals 49.7% w/w content of ligand in TFA salt following deprotection. (B). Spectrophotometric titration of picaga-DUPA with Cu²⁺. UV-Vis absorbance spectra of picaga-DUPA upon Cu²⁺ addition (left) and UV-vis titration to endpoint to determine ligand concentration (right). Analysis reveals 79.8% w/w content of ligand in TFA salt following deprotection. Due to the low sample concentration, this is considered an approximation.

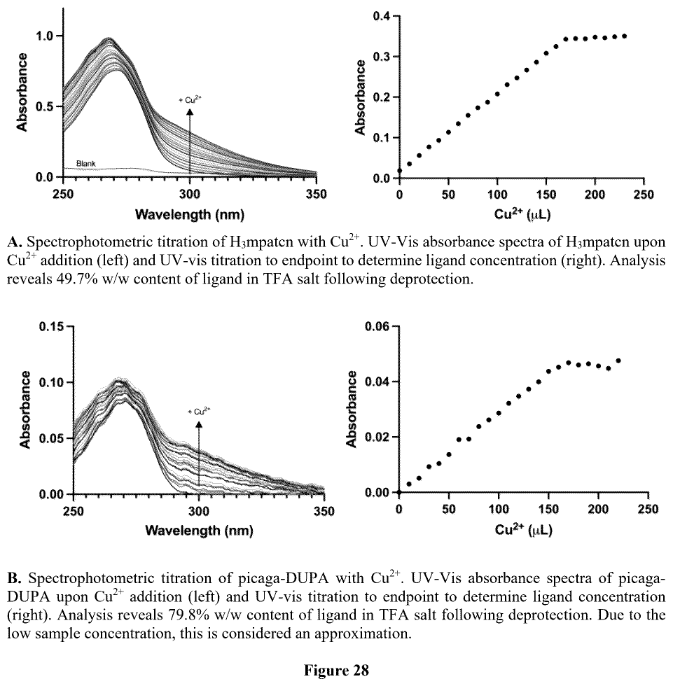

FIG. 29: (A). Concentration-dependent radioHPLC traces of the [Sc¹⁸F(mpatcn)]⁻ complex (Method C). Sc-¹⁸F retention time (t_{R})=1.18 min, [Sc¹⁸F(mpatcn)]⁻ retention time (t_{R})=2.73 min. Yields (%) are summarized in FIG. 33B. (B). Time and temperature dependent radioHPLC traces of [Sc¹⁸F(mpatcn)]⁻ in the presence and absence of 10% EtOH (Method C). Sc-¹⁸F retention time (t_{R})=1.18 min, [Sc¹⁸F(mpatcn)]⁻ retention time (t_{R})=2.73 min. Yields (%) are summarized in FIG. 33C.

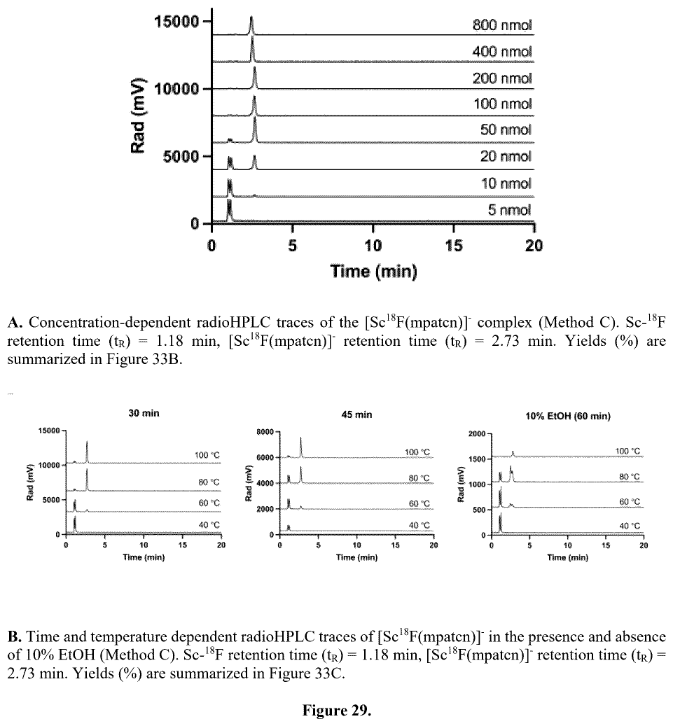

FIG. 30: HPLC trace of picaga-DUPA stacked with crude radioHPLC trace of [¹⁸F]Sc—F(picaga)-DUPA showing minor (A, retention time=5.20 min) and major (B, retention time=5.57 min) species (Method C). The major species was collected for in vivo studies.

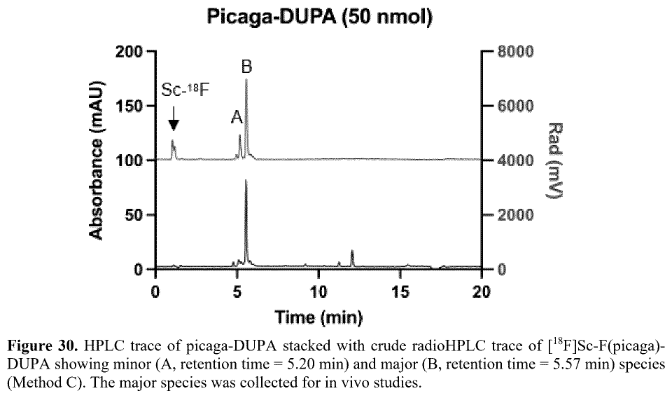

FIG. 31: Chemical structures of the clinically established [¹⁸F]-AIF-NODA, and Sc(mpatcn) derivatives discussed herein.

FIG. 32: DFT structures of a. Δ−[ScF(mpatcn)]⁻ and b. Δ-[Sc(H₂O)(mpatcn)]⁻ calculated at the B3LYP-D3 (BJ)/cc-pVDZ level of theory with SMD solvation.

FIG. 33: (A). Reaction scheme and corresponding radioHPLC trace produced by reacting the Sc-¹⁸F precursor with mpaten chelator. (B). Concentration dependent radiolabeling of 1 mCl ¹⁸F identifies an apparent molar activity of 20 mCi/μmol. (C). Temperature dependent radiolabeling of 50 nmol H₃mpaten identifies >10% yields above 60 Celsius in direct contrast with Al-¹⁸F labeling which only proceeds above 95 Celsius.

FIG. 34: (A). RadioHPLC chromatogram of [¹⁸F]Sc—F(picaga)-DUPA following purification and storage in PBS for 4 hours. No degradation or defluorination is observed. (B). Whole-body volume-rendered PET/CT in mice bearing PSMA PC3 PIP (yellow arrow) and PSMA (−) PC3 flu (white arrow) tumors shows good tumor conspicuity with no significant uptake in off-target tissues, with color scale showing standard uptake value (SUV). (C). Biodistribution analysis of [¹⁸F]Sc—F(picaga)-DUPA in direct comparison with ⁴⁷Sc-(picaga)-DUPA¹⁵ shows excellent, direct correlation of biodistribution data using both isotopes.

FIG. 35: HRMS of compound 4a. HRMS calc. for C₂₃H₃₁N₅O₉P: 552.1854. Found: 552.1854 [M+H]⁺.

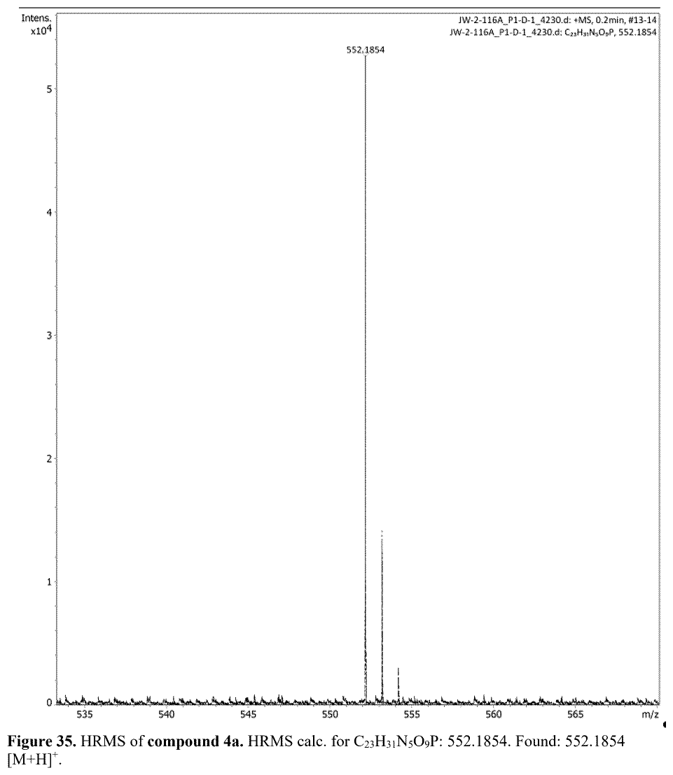

FIG. 36: HRMS of compound 4b. HRMS calc. for C₂₃H₃₁N₅O₉P: 552.1854. Found: 552.1857 [M+H]⁺.

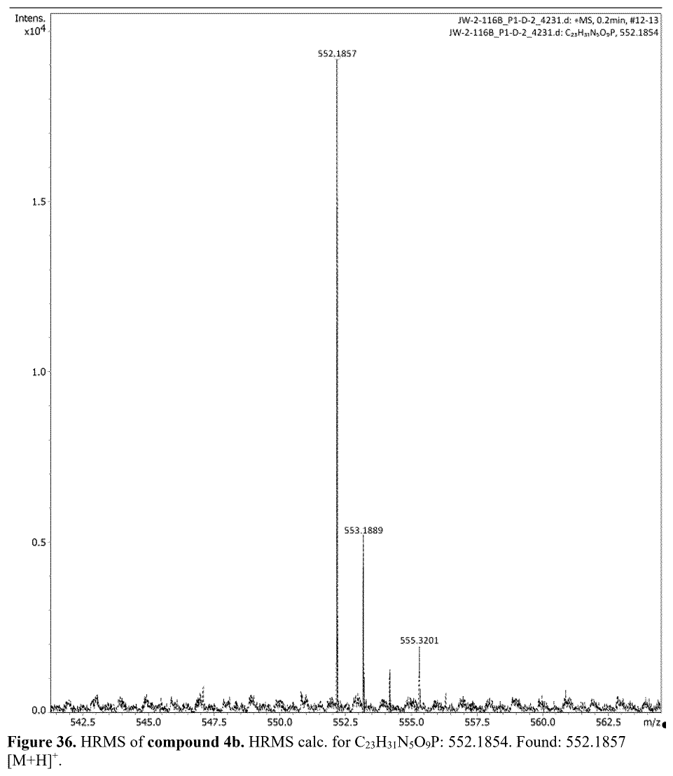

FIG. 37: (A). Full (left) and zoomed (right) HPLC chromatogram of compounds 2a-b (Method B). (B). Full (left) and zoomed (right) HPLC chromatogram of compounds 3a-b (Method B). (C). Full (left) and zoomed (right) HPLC chromatogram of compounds 4a-b (Method B).

FIG. 38: (A). ³¹P NMR of compounds 2a (12.71 ppm, black) and 2b (12.98 ppm, pink). 400 MHz in D₂O. (B). 31P NMR of compounds 3a (9.05 ppm, black) and 3b (8.91 ppm, pink). 400 MHz in CDCl₃.

FIG. 39: ³¹P NMR of compound 4a (−1.7180 ppm, black). 400 MHz in D₂O.

FIG. 40: HRMS of L3. HRMS calc. for C₁₆H₂₆N₄O₇P: 417.1534. Found: 417.1528 [M+H]⁺.

FIG. 41: (A). HPLC of 6-((4-(carboxymethyl)-7-(phosphonomethyl)-1,4,7-triazonan-1-yl)methyl)picolinic acid (L3). Retention time (t_{R})=1.97 minutes (Method B). (B). ³¹P NMR 6-((4-(carboxymethyl)-7-(phosphonomethyl)-1,4,7-triazonan-1-yl)methyl)picolinic acid (L3). 400 MHZ, CD₃OD.

### DETAILED DESCRIPTION OF THE INVENTION

The present invention provides a compound having the structure:

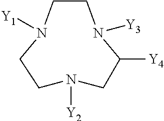

wherein
Y₁ and Y₂ are each, independently, —H, alkylheteroaryl, alkyl-CO₂H, alkylaryl-CO₂H,
   alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂, or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃; and
Y₃ and Y₄ are each, independently, —H, Z₁-L(A), Z₁-L(A)(B), L(A), L(A)(B),

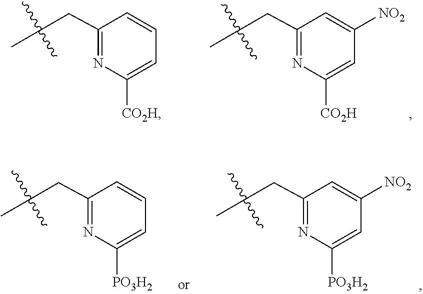

wherein
Z₁ is

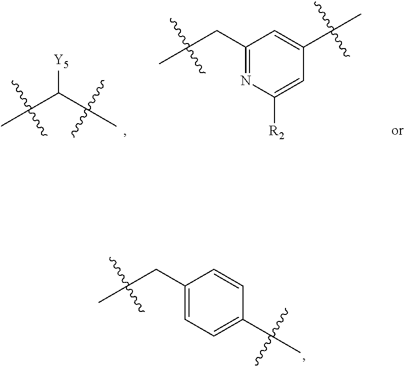

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not

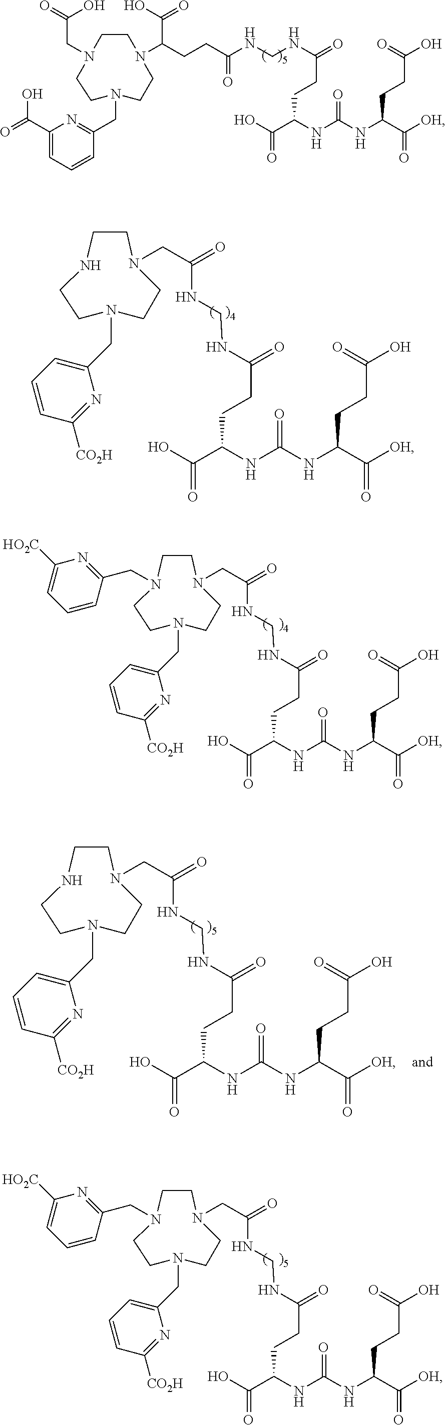

or a pharmaceutically acceptable salt of the compound.

In some embodiments, Y₃ is Z₁-L(A), Z₁-L(A)(B), L(A) or L(A)(B) and Y₄ is —H.

In some embodiments, Y₁ and Y₂ are each, independently, —H, alkyl-CO₂H, alkylaryl-CO₂H, alkylheteroaryl-CO₂H, alkylheteroaryl-(NO₂)CO₂H, alkyl-P(O)(OH)₂, alkylaryl-P(O)(OH)₂, alkylheteroaryl-P(O)(OH)₂ or alkylheteroaryl-(NO₂) P(O)(OH)₂.

In some embodiments, Y₁ and Y₂ are each, independently, alkyl-CO₂H, alkylaryl-CO₂H, alkylheteroaryl-CO₂H, alkylheteroaryl-(NO₂)CO₂H, alkyl-P(O)(OH)₂, alkylaryl-P(O)(OH)₂, alkylheteroaryl-P(O)(OH)₂ or alkylheteroaryl-(NO₂) P(O)(OH)₂, Y₃ is Z₁-L(A) or Z₁-L(A)(B) and Y₄ is —H.

In some embodiments, the heteroaryl is pyridyl.

In some embodiments, the present invention provides a compound having the structure:

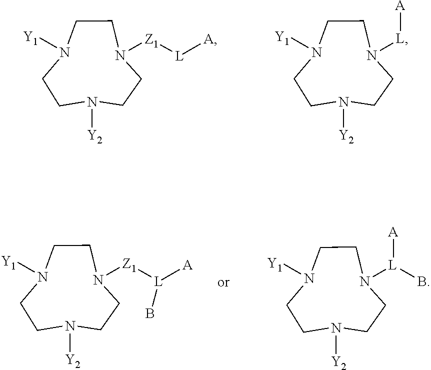

In some embodiments, the present invention provides a compound having the structure:

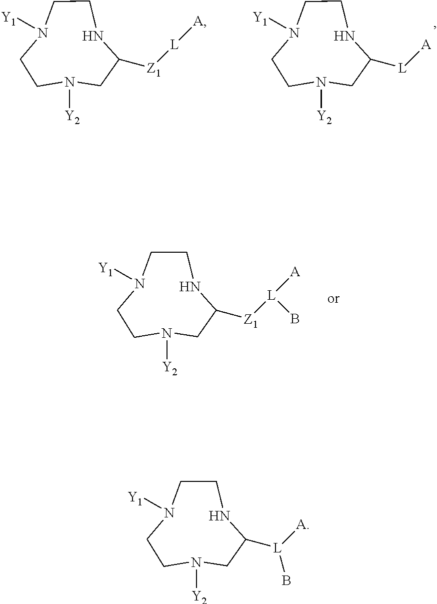

In some embodiments,
Z₁ is

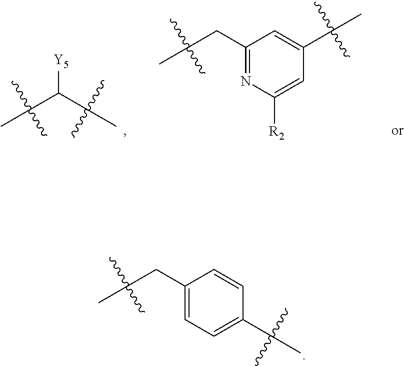

In some embodiments, the targeting moiety A is a moiety with specificity for a target protein on the surface of a cell.

In some embodiments, the targeting moiety A is a moiety with specificity for a target antigen on the surface of a cell.

In some embodiments, the targeting moiety A is a small molecule, a peptide, a protein or an antibody or a derivative or fragment thereof.

In some embodiments, the targeting moiety A is ((5-(2-(4-(aminomethyl)cyclohexane-1-carboxamido)-3-(naphthalen-2-yl)propanamido)-1-carboxypentyl)carbamoyl)glutamic acid or a derivative or fragment thereof.

In some embodiments, the targeting moiety A is 2-[3-(1,3-dicarboxypropyl)ureido]pentanedioic acid (DUPA) or a derivative or fragment thereof.

In some embodiments, the targeting moiety A is trastuzumab, bombesin or somatostatin or a derivative or fragment thereof.

In some embodiments, the targeting moiety A is covalently attached to the chemical linker L.

In some embodiments, the bond between the targeting moiety A and the chemical linker L is formed by reacting a first terminal reactive group on the targeting moiety A with a second terminal reactive group on the chemical linker L.

In some embodiments, the bond between the targeting moiety A and the chemical linker L is formed by reacting an amine moiety on the targeting moiety A with a carboxylic acid moiety on the chemical linker L.

In some embodiments, the bond between the targeting moiety A and the chemical linker L is formed by reacting a carboxylic acid moiety on the targeting moiety A with an amine moiety on the chemical linker L.

In some embodiments, the chemical linker L is an alkyl, alkenyl, alkynyl, alkylether, alkylthioether, alkylamino, alkylamido, alkylester, alkylaryl, alklyheteroaryl, aryl, heteroaryl, a natural amino acid, an unnatural amino acid, a disulfide or thioether containing linker or combinations thereof.

In some embodiments, the present invention provides a compound having the structure:

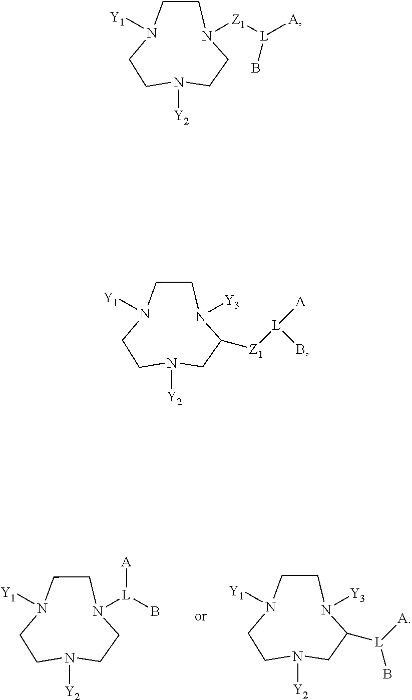

In some embodiments, the albumin-binding moiety B is a small molecule, a peptide, a protein or an antibody or a derivative or fragment thereof.

In some embodiments, the albumin-binding moiety B is 4-(4-iodophenyl)butanoic acid or a derivative or fragment thereof.

In some embodiments, the albumin-binding moiety B is 4-(4-methyl)butanoic acid or a derivative or fragment thereof.

In some embodiments, the albumin-binding moiety B is covalently attached to the chemical linker L.

In some embodiments, both the targeting moiety A and the albumin-binding moiety B are both covalently attached to the chemical linker L.

In some embodiments, the bond between the albumin-binding moiety B and the chemical linker L is formed by reacting a first terminal reactive group on the albumin-binding moiety B with a second terminal reactive group on the chemical linker L.

In some embodiments, the bond between the albumin-binding moiety B and the chemical linker L is formed by reacting a carboxylic acid moiety on the albumin-binding moiety B with an amine moiety on the chemical linker L.

In some embodiments, the bond between the albumin-binding moiety B and the chemical linker L is formed by reacting an amine moiety on the albumin-binding moiety B with a carboxylic acid moiety on the chemical linker L.

In some embodiments, L has the structure:

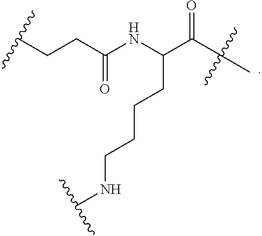 [SMILES: *CCC(=O)NC(CCCCN*)C(*)=O]

In some embodiments, the chemical linker L is a releasable linker.

In some embodiments, the chemical linker L is a non-releasable linker.

In some embodiments, the present invention provides a compound having the structure:

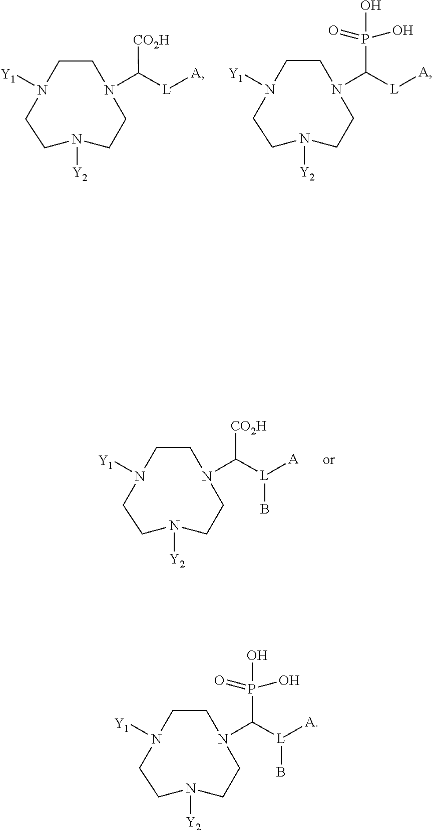

In some embodiments, the present invention provides a compound having the structure:

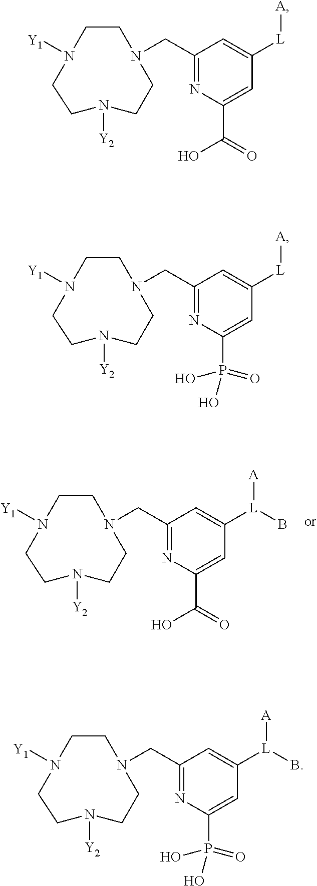

In some embodiments, the present invention provides a compound having the structure:

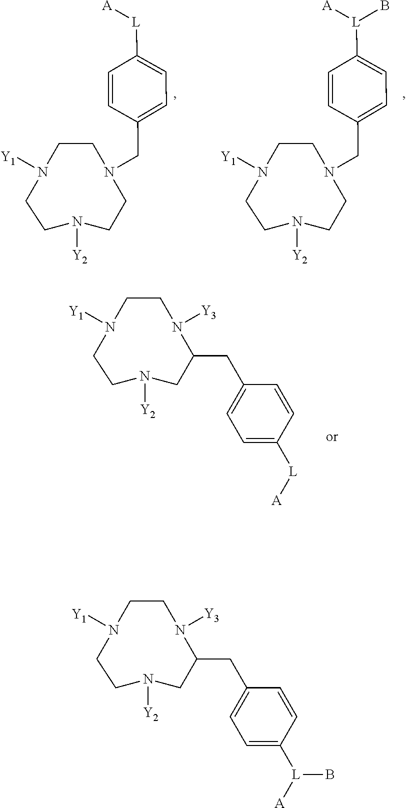

In some embodiments, Y₁ and Y₂ are each, independently, —H, alkyl-CO₂H, alkylaryl-CO₂H, alkylheteroaryl-CO₂H, alkylheteroaryl-(NO₂)(CO₂H), alkyl-P(O)(OH)₂, alkylaryl-P(O)(OH)₂, alkylheteroaryl-P(O)(OH)₂ or alkylheteroaryl-(NO₂)(P(O)(OH)₂).

In some embodiments, one of Y¹ or Y₂ is —H, or each of Y₁ and Y₂ is —H.

In some embodiments, one of Y¹ or Y₂ is alkyl-CO₂H, or each of Y₁ and Y₂ is alkyl-CO₂H.

In some embodiments, one of Y¹ or Y₂ is alkylaryl-CO₂H or alkylheteroaryl-CO₂H, or each of Y₁ and Y₂ is alkylaryl-CO₂H or alkylheteroaryl-CO₂H.

In some embodiments, one of Y¹ or Y₂ is alkyl-P(O)(OH)₂, or each of Y₁ and Y₂ is alkyl-P(O)(OH)₂.

In some embodiments, one of Y¹ or Y₂ is alkylaryl-P(O)(OH)₂ or alkylheteroaryl-P(O)(OH)₂, or each of Y₁ and Y₂ is alkylaryl-P(O)(OH)₂ or alkylheteroaryl-P(O)(OH)₂.

In some embodiments, Y₁ is alkyl-CO₂H, and Y₂ is alkylaryl-CO₂H or alkylheteroaryl-CO₂H.

In some embodiments, Y₁ is alkyl-CO₂H, and Y₂ is alkylaryl-P(O)(OH)₂ or alkylheteroaryl-P(O)(OH)₂.

In some embodiments, Y₁ is alkyl-P(O)(OH)₂, and Y₂ is alkylaryl-CO₂H or alkylheteroaryl-CO₂H.

In some embodiments, the heteroaryl is pyridyl.

In some embodiments, the present invention provides a compound having the structure:

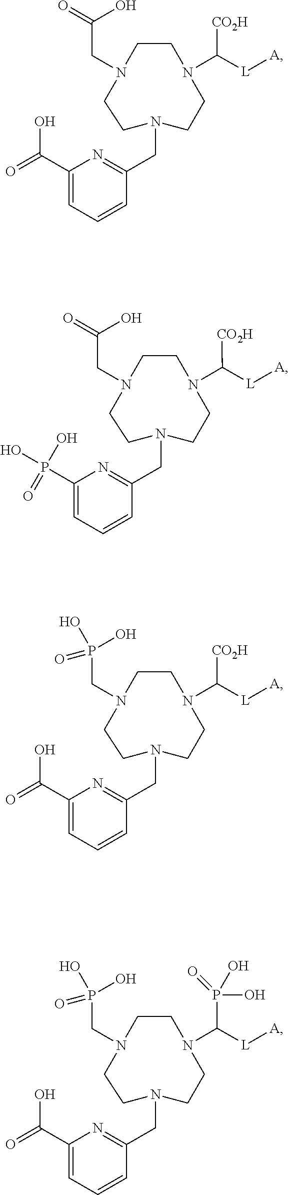

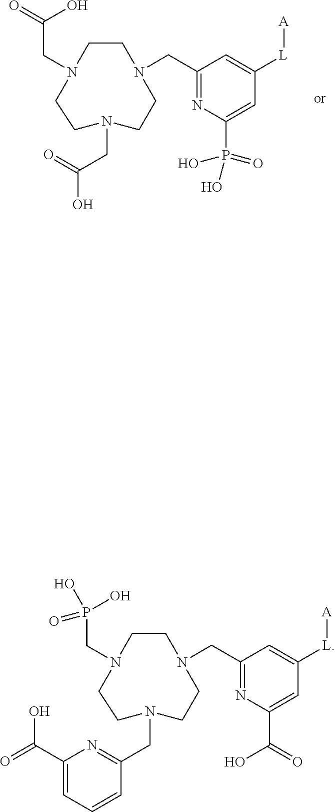

In some embodiments, the present invention provides a compound having the structure:

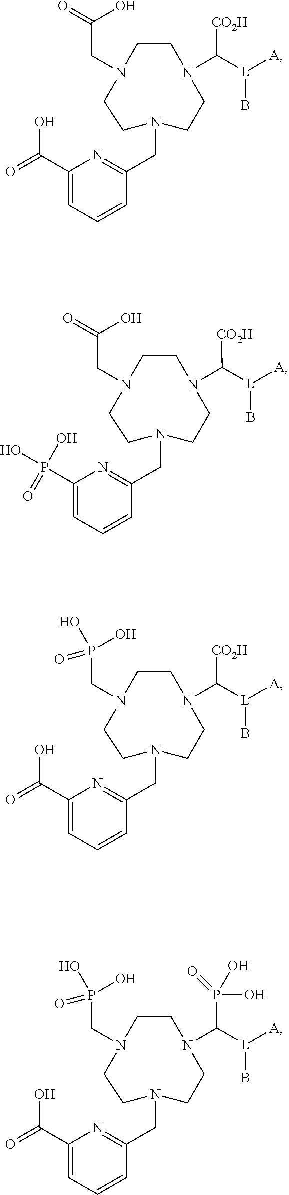

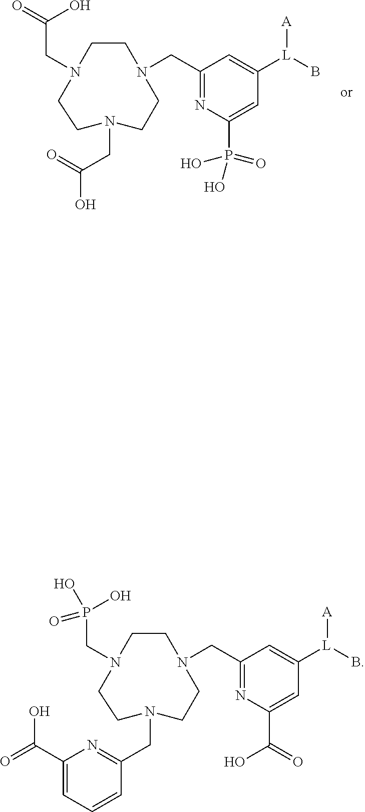

In some embodiments, the present invention provides a compound having the structure:

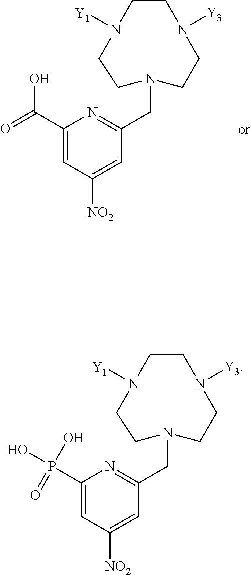

The present invention also provides a pharmaceutical composition comprising the compound of the present invention and a pharmaceutically acceptable carrier.

In some embodiments, a metal complex comprising the compound of the present invention, wherein the compound coordinates or chelates or complexes to a metal or metal-ion (M).

In some embodiments, the metal is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb).

In some embodiments, the metal-ion is Scandium-Fluorine-18 (^{nat}Sc-¹⁸F), Lanthanum-Fluorine-18 (^{nat}La-¹⁸F), or Lutetium-Fluorine-18 (^{nat}Lu-¹⁸F).

In some embodiments, the metal-ion is Scandium-44-Fluorine (⁴⁴Sc—F), Scandium-47-Fluorine (⁴⁷Sc—F), Lanthanum-132-Fluorine (¹³²La—F), Lanthanum-135-Fluorine (¹³⁵La—F) or Lutetium-177-Fluorine (¹⁷⁷Lu—F).

In some embodiments, the Fluorine is Fluorine-18 (¹⁸F).

In some embodiments, the present invention provides a metal complex having the structure:

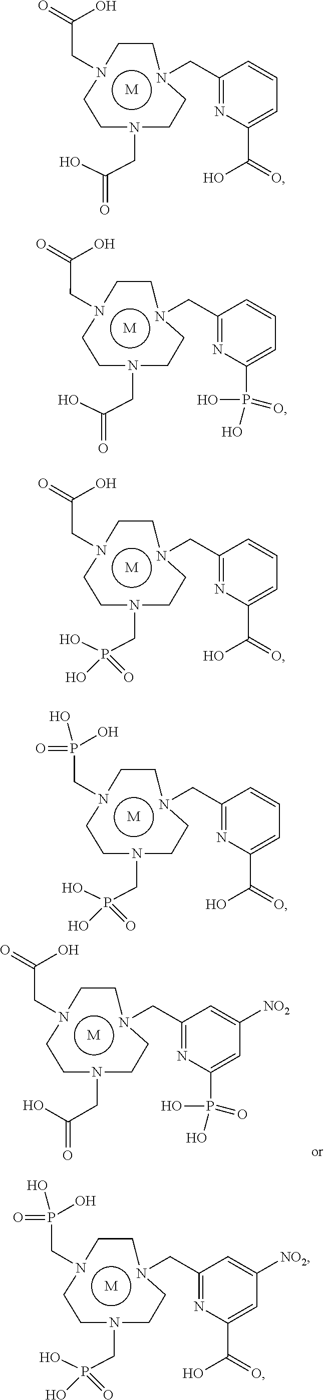

or a pharmaceutically acceptable salt thereof.

In some embodiments, the present invention provides a metal complex having the structure:

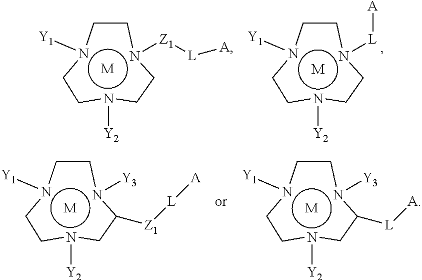

or a pharmaceutically acceptable salt thereof.

In some embodiments, the present invention provides a metal complex having the structure:

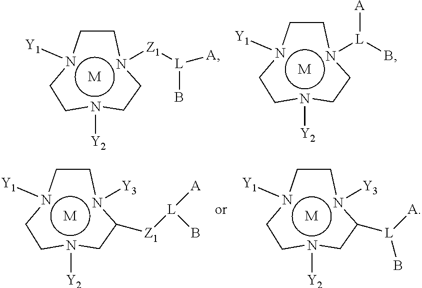

or a pharmaceutically acceptable salt thereof.

In some embodiments, the present invention provides a metal complex having the structure:

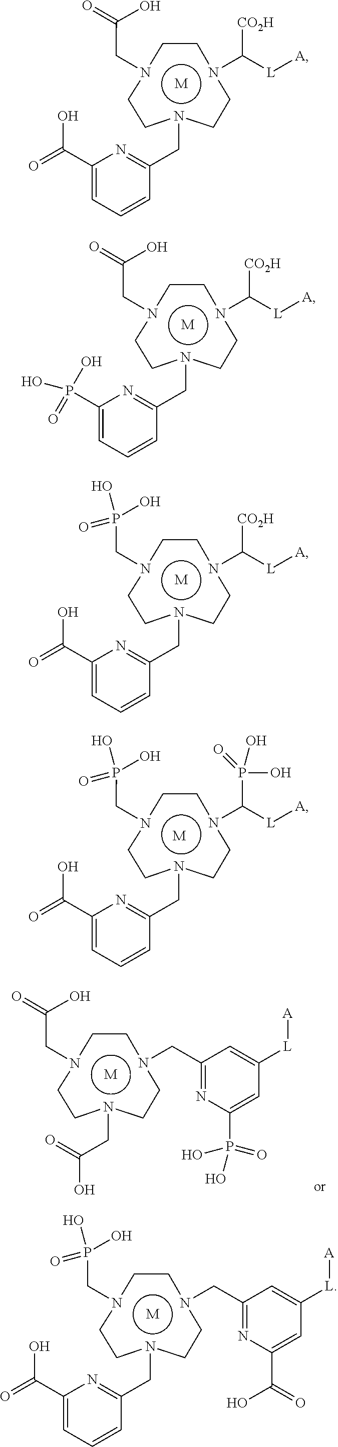

In some embodiments, the present invention provides a metal complex having the structure:

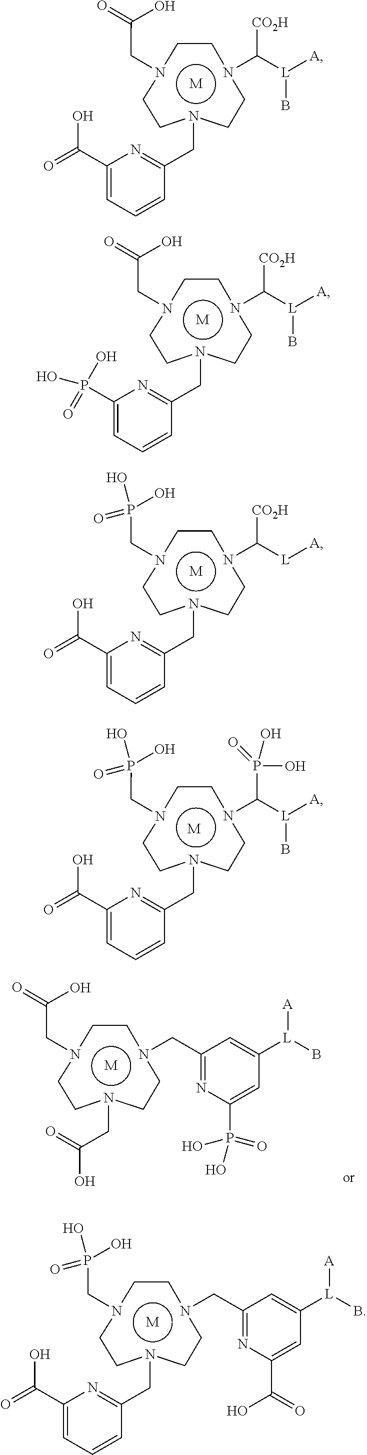

In some embodiments, a pharmaceutical composition comprising the metal complex of the present invention and a pharmaceutically acceptable carrier.

The present invention provides a method of detecting target cells in a subject comprising administering an effective amount of the metal complex of the present invention or the composition of the present invention to the subject, and imaging the subject with a molecular imaging device to detect the metal complex or composition in the subject.

In some embodiments, the target cells are cancer cells.

The present invention provides a method of imaging target cells in a subject comprising:
1) administering to the subject an effective amount of the metal complex of the present invention or a pharmaceutically acceptable salt thereof, or the composition of the present invention,
   wherein the compound specifically accumulates at the target cells in the subject;
2) detecting in the subject the location of the metal complex or the composition; and
3) obtaining an image of the target cells in the subject based on the location of the metal complex or the composition in the subject.

The present invention provides a method of detecting the presence of target cells in a subject which comprises determining if an amount of the metal complex of the present invention or a pharmaceutically acceptable salt thereof, or the composition of the present invention is present in the subject at a period of time after administration of the metal complex or composition to the subject, thereby detecting the presence of the target cells based on the amount of the metal complex or composition determined to be present in the subject.

In some embodiments, the detecting is performed by a Positron Emission Tomography (PET) device.

In some embodiments, the detecting is performed by a Single-Photon Emission Computed Tomography (SPECT) device.

In some embodiments, A has the structure:

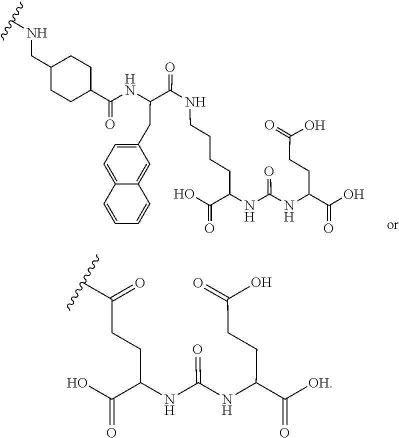 [SMILES: *NCC1CCC(C(=O)NC(Cc2ccc3ccccc3c2)C(=O)NCCCCC(NC(=O)NC(CCC(=O)O)C(=O)O)C(=O)O)CC1 ; *C(=O)CCC(NC(=O)NC(CCC(=O)O)C(=O)O)C(=O)O]

In some embodiments, the present invention provides a compound having the structure:

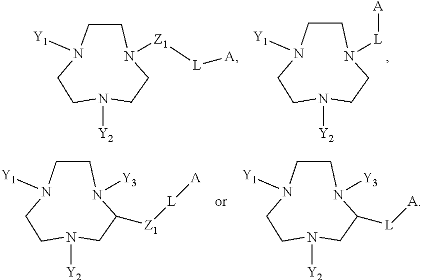

In some embodiments, the present invention provides a compound having the structure:

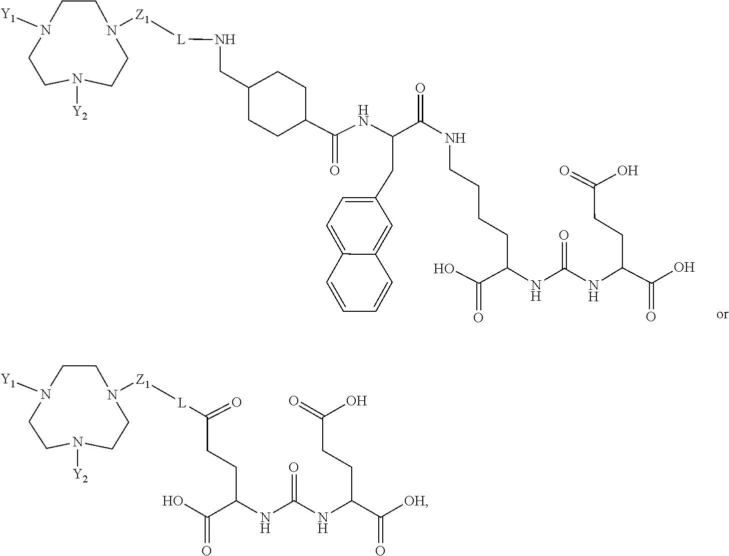

Y₁ and Y₂ are each, independently, —H, alkylheteroaryl, alkyl-CO₂H, alkylaryl-CO₂H,
   alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂ or alkylheteroaryl-(NO₂)(P(O)(OH)₂),
      wherein each occurrence of R₁ is, independently, —H, alkyl, alkenyl, alkynyl, alkyl-aryl, alkyl-heteroaryl,
      aryl, heteroaryl, alkyl-CF₃ or —Si(alkyl)₃;
Z₁ is

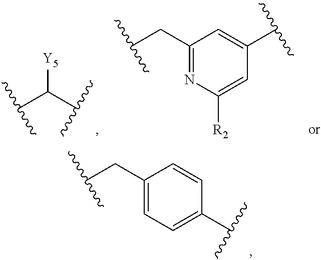

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂; and
L is a chemical linker,
   or a pharmaceutically acceptable salt of the compound.

In some embodiments, the chemical linker L is an alkyl, alkenyl, alkynyl, alkylether, alkylthioether, alkylamino, alkylamido, alkylester, alkylaryl, alklyheteroaryl, aryl, heteroaryl, a natural amino acid, an unnatural amino acid, a disulfide or thioether containing linker or combinations thereof.

In some embodiments, the chemical linker L is alkyl, alkenyl, alkynyl, alkyl-O-alkyl, alkyl-O-alkyl-O-alkyl, alkyl-NH, alkyl-NH-alkyl, alkyl-C(O)O-alkyl, alkyl-OC(O)-alkyl alkyl-CO-alkyl, alkyl-C(O)NH-alkyl, alkyl-NHC(O)-alkyl or alkyl-C(O)NH-alkyl-NH or combinations thereof.

In some embodiments, Z₁ is

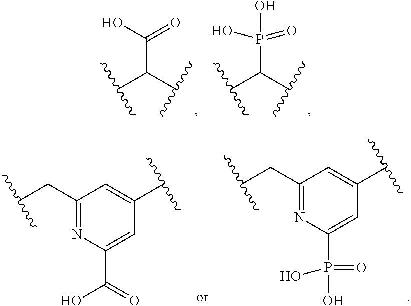 [SMILES: *C(*)C(=O)O ; *C(*)P(=O)(O)O ; *Cc1cc(*)cc(C(=O)O)n1 ; *Cc1cc(*)cc(P(=O)(O)O)n1]

In some embodiments, Y₁ and Y₂ are each, independently, —H, alkyl-CO₂H, alkylaryl-CO₂H, alkylheteroaryl-CO₂H, alkylheteroaryl-(NO₂)(CO₂H), alkyl-P(O)(OH)₂, alkylaryl-P(O)(OH)₂, alkylheteroaryl-P(O)(OH)₂ or alkylheteroaryl-(NO₂)(P(O)(OH)₂).

In some embodiments, Y₁ and Y₂ are each, independently, —H,

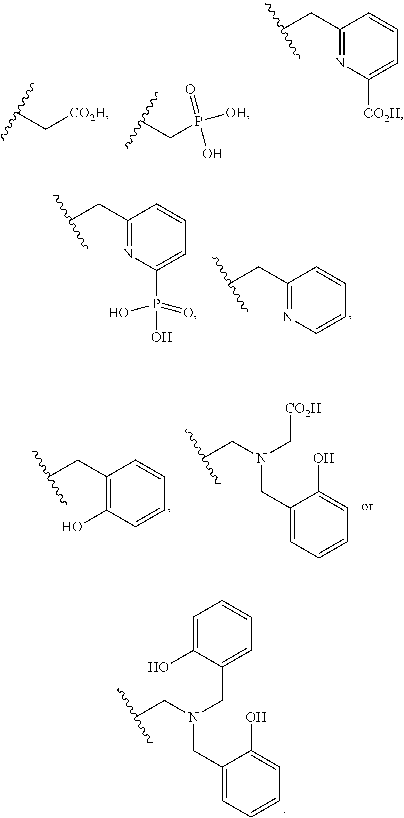

In some embodiments, the present invention provides a compound having the structure:

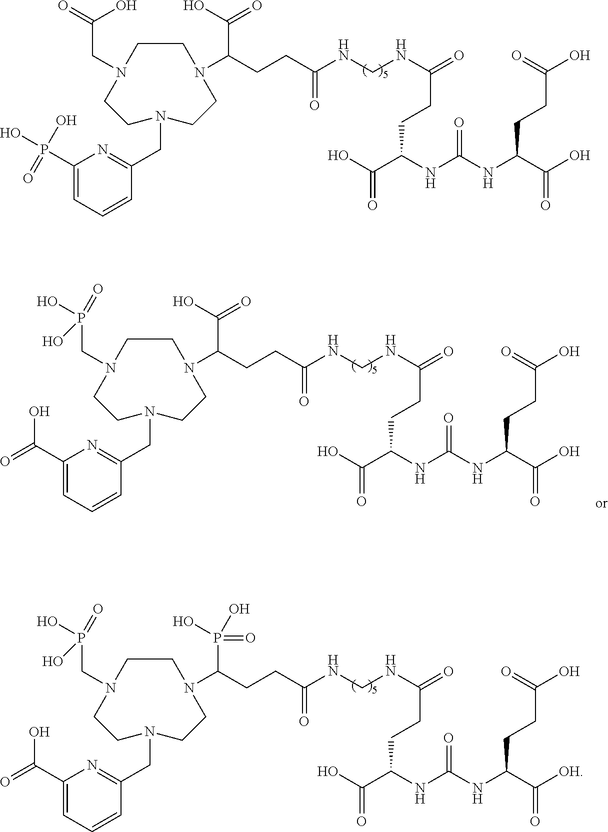

In some embodiments, the present invention provides a compound having the structure:

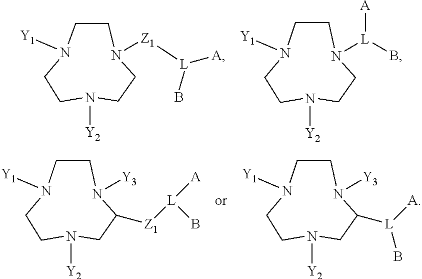

In some embodiments, the present invention provides a compound having the structure:

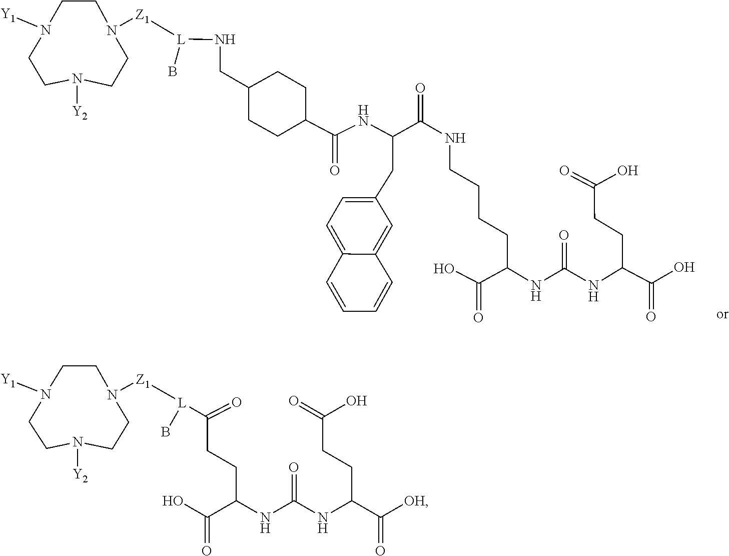

wherein
Y₁ and Y₂ are each, independently, —H, alkylheteroaryl, alkyl-CO₂H, alkylaryl-CO₂H,
   alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂ or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃;
Z₁ is

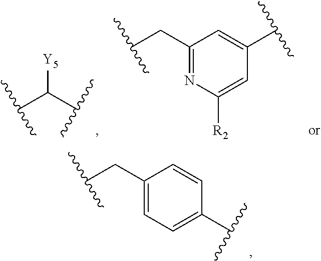

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂;
L is a chemical linker; and
B is an albumin-binding moiety,
   or a pharmaceutically acceptable salt of the compound.

In some embodiments, the chemical linker L is an alkyl, alkenyl, alkynyl, alkylether, alkylthioether, alkylamino, alkylamido, alkylester, alkylaryl, alklyheteroaryl, aryl, heteroaryl, a natural amino acid, an unnatural amino acid, a disulfide or thioether containing linker or combinations thereof.

In some embodiments, the chemical linker L is alkyl, alkenyl, alkynyl, alkyl-O-alkyl, alkyl-O-alkyl-O-alkyl, alkyl-NH, alkyl-NH-alkyl, alkyl-C(O)O-alkyl, alkyl-OC(O)-alkyl alkyl-CO-alkyl, alkyl-C(O)NH-alkyl, alkyl-NHC(O)-alkyl, alkyl-C(O)NH-alkyl-NH, alkyl-C(O)NH-(alkyl-C(O)) (alkyl-NH) or combinations thereof.

In some embodiments, L has the structure:

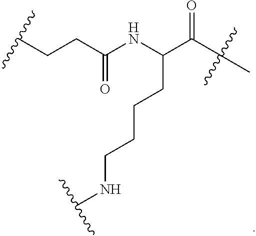 [SMILES: *CCC(=O)NC(CCCCN*)C(*)=O]

In some embodiments, the present invention provides a compound having the structure:

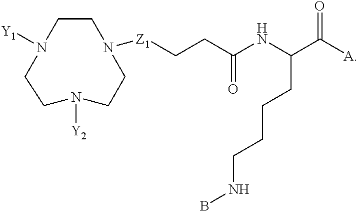

In some embodiments, Z₁ is

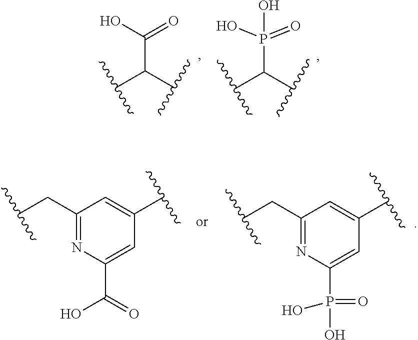 [SMILES: *C(*)C(=O)O ; *C(*)P(=O)(O)O ; *Cc1cc(*)cc(C(=O)O)n1 ; *Cc1cc(*)cc(P(=O)(O)O)n1]

In some embodiments, Y₁ and Y₂ are each, independently, —H, alkyl-CO₂H, alkylaryl-CO₂H, alkylheteroaryl-CO₂H, alkylheteroaryl-(NO₂)(CO₂H), alkyl-P(O)(OH)₂, alkylaryl-P(O)(OH)₂, alkylheteroaryl-P(O)(OH)₂ or alkylheteroaryl-(NO₂)(P(O)(OH)₂).

In some embodiments, Y₁ and Y₂ are each, independently, —H,

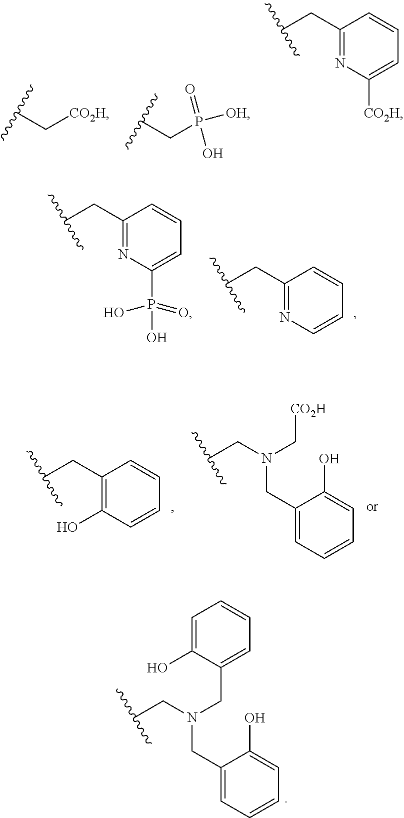

In some embodiments, B has the structure:

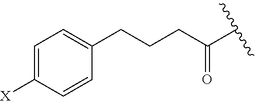

wherein
X is halogen or alkyl.

In some embodiments, the halogne is F, Br, I.

In some embodiments, the halogne is I.

In some embodiments, the present invention provides a compound having the structure:

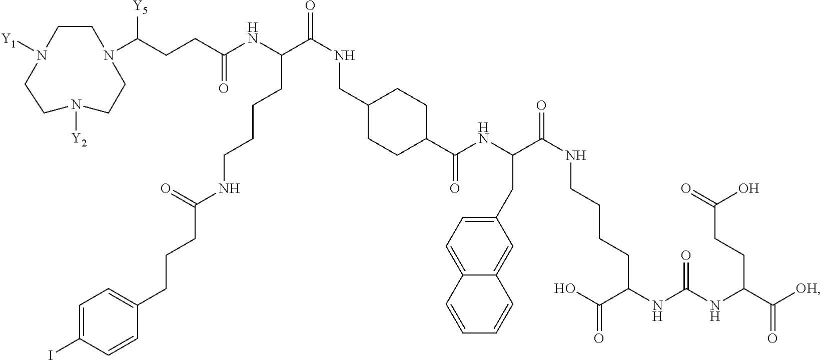

wherein
Y₁ and Y₂ are each, independently —H,

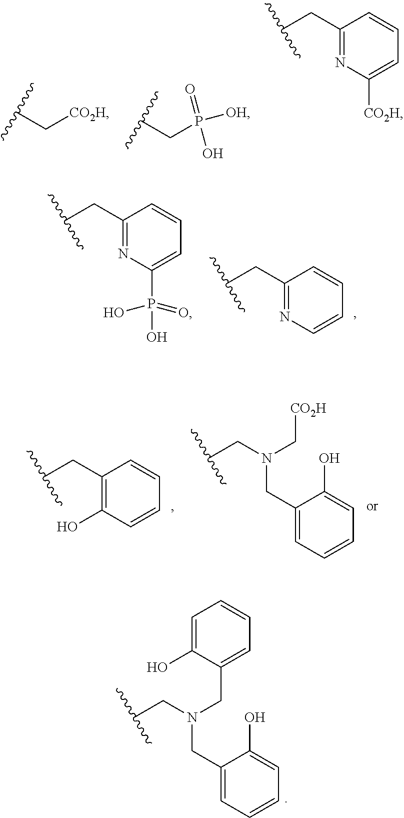

and
Y₅ is —CO₂H or —P(O)(OH)₂,
   or a pharmaceutically acceptable salt of the compound.

In some embodiments, the present invention provides a compound having the structure:

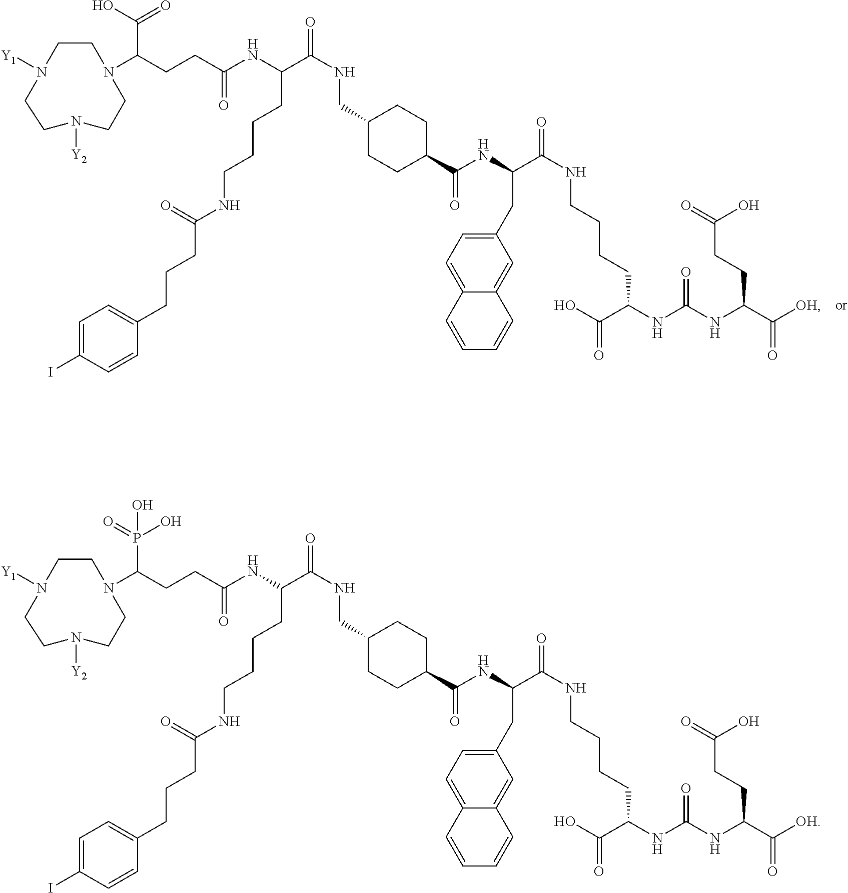

In some embodiments, Y₁ and Y₂ are each, independently

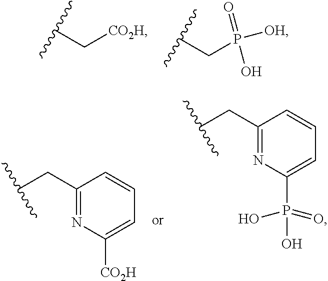

or a pharmaceutically acceptable salt of the compound.

In some embodiments, the present invention provides a compound having the structure:

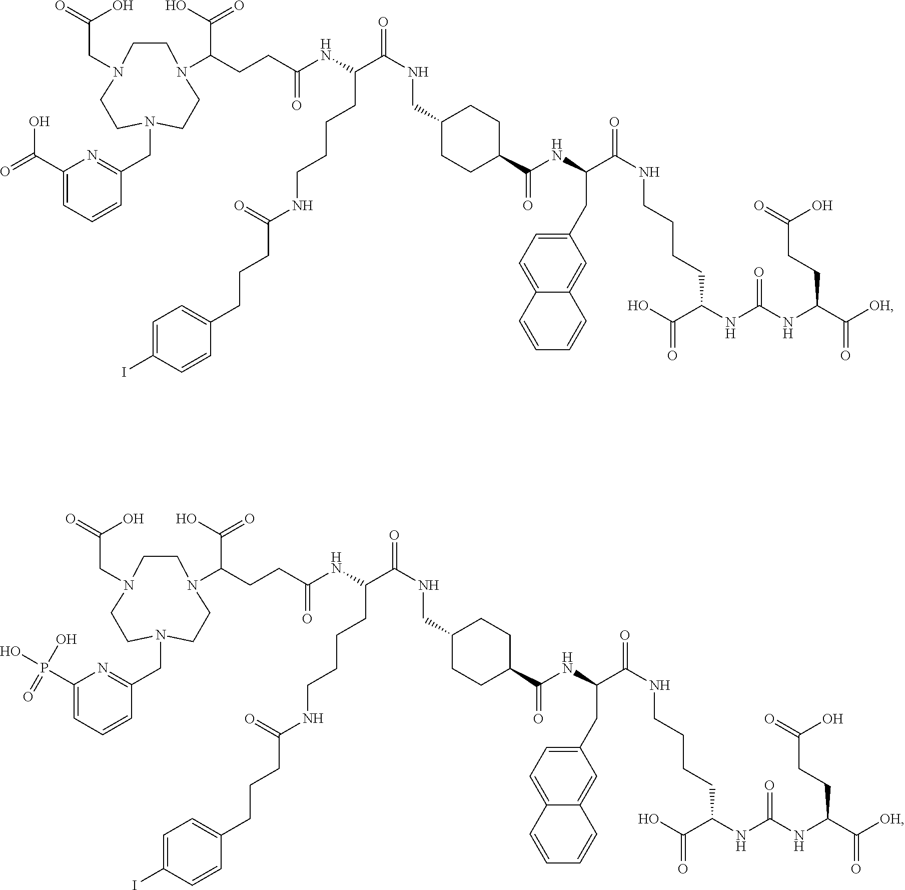

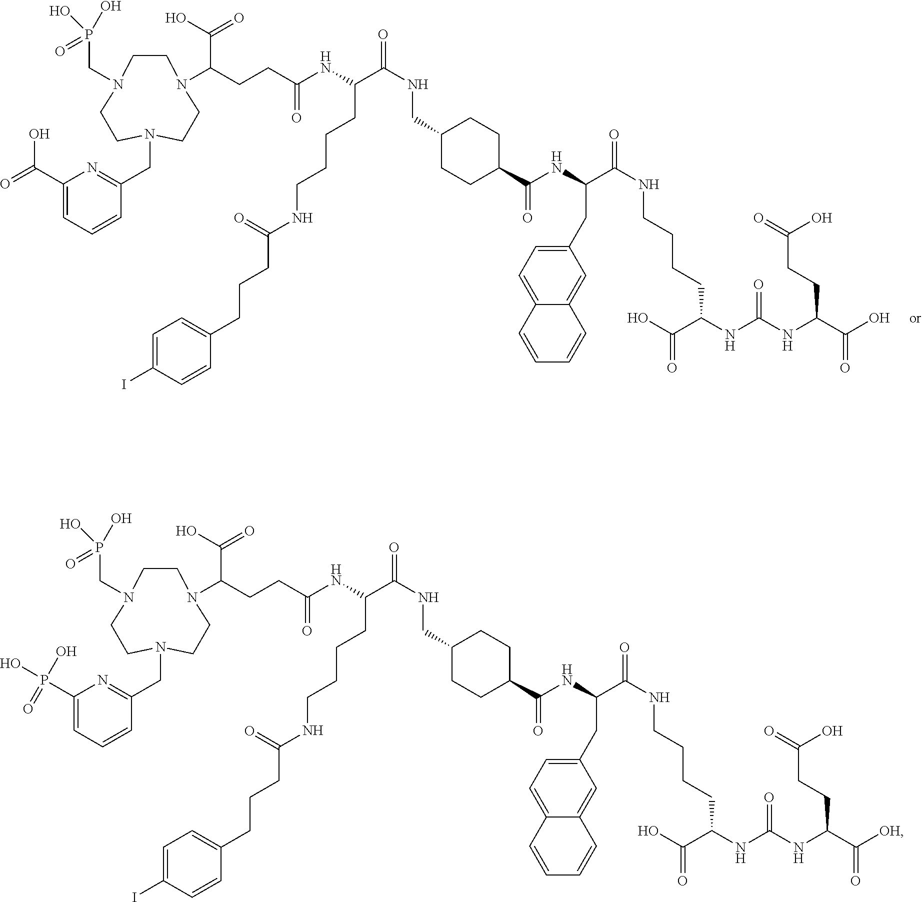

or a pharmaceutically acceptable salt of the compound.

In some embodiments, the present invention provides a pharmaceutical composition comprising the compound of the present invention and a pharmaceutically acceptable carrier.

In some embodiments, the present invention provides a metal complex comprising the compound of the present invention, wherein the compound coordinates or chelates or complexes to a metal or metal-ion (M).

In some embodiments, the metal is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb).

In some embodiments, the metal-ion is Scandium-Fluorine-18 (^{nat}Sc-¹⁸F), Lanthanum-Fluorine-18 (^{nat}La-¹⁸F), or Lutetium-Fluorine-18 (^{nat}Lu-¹⁸F).

In some embodiments, the metal-ion is Scandium-44-Fluorine (⁴⁴Sc—F), Scandium-47-Fluorine (⁴⁷Sc—F), Lanthanum-132-Fluorine (¹³²La—F), Lanthanum-135-Fluorine (¹³⁵La—F) or Lutetium-177-Fluorine (¹⁷⁷Lu—F).

In some embodiments, wherein the Fluorine is Fluorine-18 (¹⁸F).

In some embodiments, the present invention provides a metal complex having the structure:

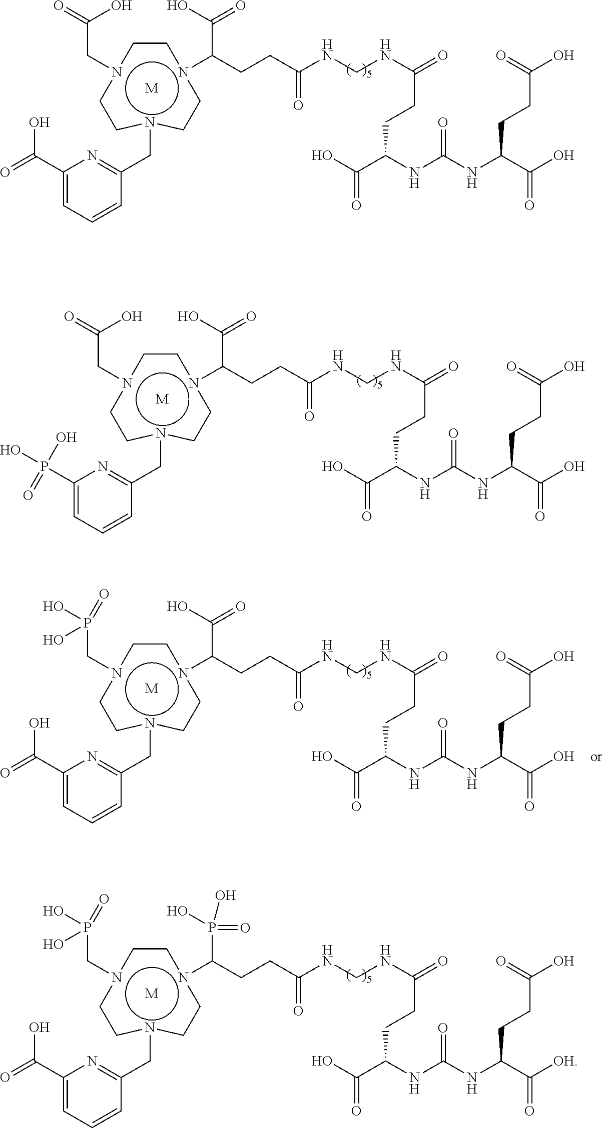

In some embodiments, the present invention provides a metal complex having the structure:

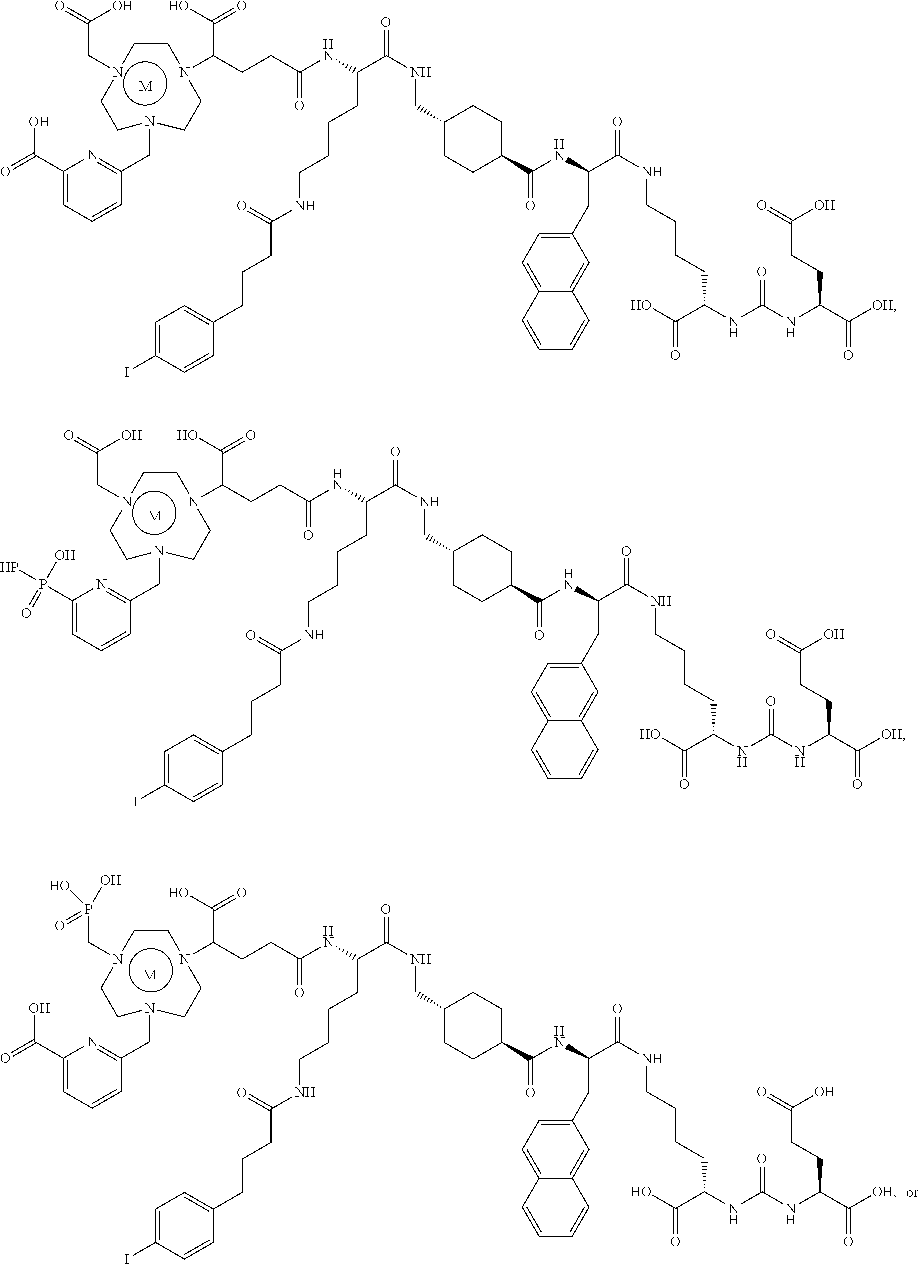

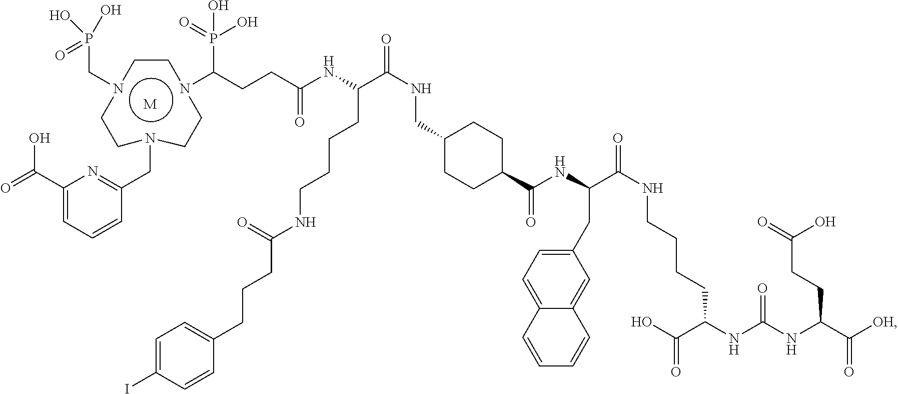

or a pharmaceutically acceptable salt of the compound.

In some embodiments, the present invention provides a pharmaceutical composition comprising the metal complex of the present invention and a pharmaceutically acceptable carrier.

The present invention also provides a method of detecting cancer cells in a subject comprising administering an effective amount of the metal complex of the present invention or the composition of the present invention to the subject, and imaging the subject with a molecular imaging device to detect the metal complex or composition in the subject, wherein the cancer cells are prostate cancer cells, wherein the cancer cells have elevated levels of prostate-specific membrane antigen (PSMA).

The present invention provides a method of imaging prostate cancer cells in a subject comprising:
1) administering to the subject an effective amount of the metal complex of the present invention or a pharmaceutically acceptable salt thereof, or the composition of the present invention,
   wherein the compound specifically accumulates at prostate cancer cells in the subject;
2) detecting in the subject the location of the metal complex or the composition; and
3) obtaining an image of the cancer cells in the subject based on the location of the metal complex or the composition in the subject.

The present invention provides a method of detecting the presence of prostate cancer cells in a subject which comprises determining if an amount of the metal complex of the present invention or a pharmaceutically acceptable salt thereof, or the composition of the present invention is present in the subject at a period of time after administration of the metal complex or composition to the subject, thereby detecting the presence of the prostate cancer cells based on the amount of the metal complex or composition determined to be present in the subject.

The present invention provides a method of reducing the size of a prostate tumor or of inhibiting proliferation of prostate cancer cells comprising contacting the tumor or cancer cells with the metal complex of the present invention or a pharmaceutically acceptable salt thereof, or the composition of the present invention, thereby reducing the size of the tumor or inhibit proliferation of the cancer cells.

The present invention also provides a process for producing a metal complex having the structure:

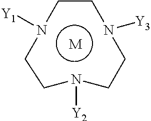

wherein
M is ^{nat}Sc-¹⁸F, ⁴⁴Sc-¹⁸F, ⁴⁷Sc-¹⁸F, ^{nat}La-¹⁸F, ¹³²La-¹⁸F, ¹³⁵La-¹⁸F, ^{nat}Lu-¹⁸F or ¹⁷⁷Lu-¹⁸F,
   comprising
   (a) contacting the compound having the structure:

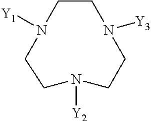

with a preformed M complex in a first suitable solvent to produce a metal complex having the structure:

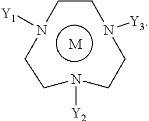

In some embodiments, the present invention provides a peptide consists of between 1-500 residues, wherein the residues can be natural and unnatural amino acids, and wherein the amino acids may be linear, cyclic and bicyclic.

The present invention provides a method of detecting target cells in a subject comprising administering an effective amount of the metal complex of the present invention or the composition of the present invention to the subject, and imaging the subject with a molecular imaging device to detect the metal complex or composition in the subject.

In some embodiments, the compound or composition specifically accumulates at the target cells.

In some embodiments, the target cells are cancer cells.

In some embodiments, the target cells are prostate cancer cells.

In some embodiments, a detection of the compound or composition in the target cells of the subject is an indication that cancers cells are present in subject.

In some embodiments, the compound or composition is detected using a PET imaging device.

The present invention provides a method of imaging target cells in a subject comprising:
1) administering to the subject an effective amount of the metal complex of the present invention or a pharmaceutically acceptable salt thereof, or the composition of the present invention,
   wherein the compound specifically accumulates at the target cells in the subject;
2) detecting in the subject the location of the metal complex or the composition; and
4) obtaining an image of the target cells in the subject based on the location of the metal complex or the composition in the subject.

In some embodiments, the compound or composition is detected using a PET imaging device.

In some embodiments, the image obtained is a three-dimensional image.

The present invention provides a method of detecting the presence of target cells in a subject which comprises determining if an amount of the metal complex of the present invention or a pharmaceutically acceptable salt thereof, or the composition of the present invention is present in the subject at a period of time after administration of the metal complex or composition to the subject, thereby detecting the presence of the target cells based on the amount of the metal complex or composition determined to be present in the subject.

In some embodiments, the detecting is performed by a Positron Emission Tomography (PET) device.

In some embodiments, the detecting is performed by a Single-Photon Emission Computed Tomography (SPECT) device.

In some embodiments, the method further comprising quantifying the amount of the compound in the subject and comparing the quantity to a predetermined control.

In some embodiments, the method further comprising determining whether the subject is afflicted with cancer based on the amount of the compound in the subject.

In some embodiments, the method further comprising determining the stage of the cancer.

The present invention provides a method of reducing the size of a tumor or of inhibiting proliferation of cancer cells comprising contacting the tumor or cancer cells with the metal complex of the present invention or a pharmaceutically acceptable salt thereof, or the composition of the present invention, so as to thereby reducing the size of the tumor or inhibit proliferation of the cancer cells.

In some embodiments, chemical linker L is C₂-C₁₂ alkyl, C₂-C₁₂ alkyl-NH, C₂-C₁₂ alkyl-NHC(O)—C₂-C₁₂ alkyl, C₂-C₁₂ alkyl-C(O)NH—C₂-C₁₂ alkyl or C₂-C₁₂ alkyl-C(O)NH—C₂-C₁₂ alkyl-NH.

In some embodiments, chemical linker L is C₄-alkyl-NH.

In some embodiments, chemical linker L is C₅-alkyl-NH.

In some embodiments, chemical linker L is C₂-alkyl-C(O)NH—C₄ alkyl-NH or C₂-alkyl-C(O)NH—C₅ alkyl-NH.

In some embodiments, chemical linker L is C₄-alkyl-NH or C₅-alkyl-NH.

In some embodiments, chemical linker L is C₂-alkyl-C(O)NH—C₄ alkyl-NH or C₂-alkyl-C(O)NH—C₅ alkyl-NH.

In some embodiments, chemical linker L is C₂-C₁₂ alkyl, C₂-C₁₂ alkyl-NH, C₂-C₁₂ alkyl-NHC(O)—C₂-C₁₂ alkyl, C₂-C₁₂ alkyl-C(O)NH—C₂-C₁₂ alkyl or C₂-C₁₂ alkyl-C(O)NH—C₂-C₁₂ alkyl-NH.

In some embodiments, chemical linker L is

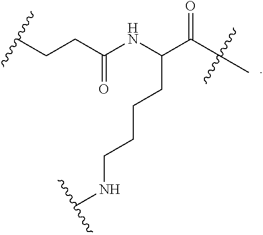 [SMILES: *CCC(=O)NC(CCCCN*)C(*)=O]

In some embodiments, chemical linker L is

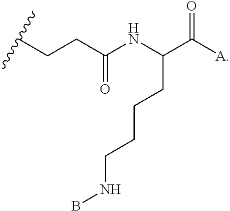

In some embodiments, each of Y₁ and Y₂ is

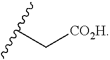 [SMILES: *CC(=O)O]

In some embodiments, the present invention provides a metal complex comprising the compound of the present invention, wherein the compound coordinates to a metal.

In some embodiments, the metal is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb).

In some embodiments, the metal is Scandium-47 (⁴⁷Sc) or Copper-67 (⁶⁷Cu). Furthermore, X-Fluoride-18, where X corresponds to the metal ion bound to the chelator and may be any of the elements mentioned above in its stable (^{nat}La, ^{nat}Sc, ^{nat}Lu) or radioactive form, with Fluorine-18 or Fluorine-19 bound directly to the metal center.

In some embodiments, the present invention provides a pharmaceutical composition comprising the metal complex of the present invention and a pharmaceutically acceptable carrier.

The present invention provides a method of detecting cancer cells in a subject comprising administering an effective amount of the metal complex of the present invention or the composition of the present invention to the subject, and imaging the subject with a molecular imaging device to detect the metal complex or composition in the subject.

In some embodiments, the cancer cells are prostate cancer cells.

In some embodiments, the compound or composition specifically accumulates at prostate cancer cells.

In some embodiments, a detection of the compound or composition in the prostate gland of the subject is an indication that cancers cells are present in the prostate gland.

In some embodiments, the cancer cells have elevated levels of prostate-specific membrane antigen (PSMA).

In some embodiments, the compound or composition is detected using a PET imaging device.

In some embodiments, the compound or composition is detected using a SPECT imaging device.

The present invention provides a method of imaging prostate cancer cells in a subject comprising:
1) administering to the subject an effective amount of the metal complex of the present invention or a pharmaceutically acceptable salt thereof, or the composition of the present invention,
   wherein the compound specifically accumulates at prostate cancer cells in the subject;
2) detecting in the subject the location of the metal complex or the composition; and
3) obtaining an image of the cancer cells in the subject based on the location of the metal complex or the composition in the subject.

In some embodiments, the image obtained is a three-dimensional image.

The present invention provides a method of detecting the presence of prostate cancer cells in a subject which comprises determining if an amount of the metal complex of the present invention or a pharmaceutically acceptable salt thereof, or the composition of the present invention is present in the subject at a period of time after administration of the metal complex or composition to the subject, thereby detecting the presence of the prostate cancer cells based on the amount of the metal complex or composition determined to be present in the subject.

In some embodiments, the detecting is performed by a Positron Emission Tomography (PET) device.

In some embodiments, the compound or composition is detected using a SPECT imaging device.

In some embodiments, the method further comprising quantifying the amount of the compound in the subject and comparing the quantity to a predetermined control.

In some embodiments, the method further comprising determining whether the subject is afflicted prostate cancer based on the amount of the compound in the subject.

In some embodiments, the method further comprising determining the stage of the prostate cancer.

The present invention provides a method of reducing the size of a prostate tumor or of inhibiting proliferation of prostate cancer cells comprising contacting the tumor or cancer cells with the metal complex of the present invention or a pharmaceutically acceptable salt thereof, or the composition of the present invention, so as to thereby reducing the size of the tumor or inhibit proliferation of the cancer cells.

In some embodiments, Y₁ is —H and Y₂ is other than H.

In some embodiments, Y₁ and Y₂ are each —H.

In some embodiments, Y₁ and Y₂ are each other than —H.

In some embodiments, the present invention provides a method for reducing one or more symptoms of disease in a subject, comprising administering an effective amount of the compound of the present invention or the composition of the present invention to the subject so as to treat the disease in the subject.

In some embodiments, the disease is cancer.

In some embodiments, the cancer cells have elevated levels of proteins or antigens or both.

In some embodiments, the metal (M) is a radioisotope.

The present invention provides a pharmaceutical composition comprising a compound of the present invention and a pharmaceutically acceptable carrier.

The present invention provides a method for detecting cancer cells in a subject comprising administering an effective amount of a compound of the present invention or a composition of the present invention to the subject, and imaging the subject with a molecular imaging device to detect the compound or composition in the subject.

In some embodiments of the method, the compound or composition specifically accumulates in cancer cells relative to non-cancer cells.

In some embodiments of the method, a detection of the compound or composition in an organ of the subject is an indication that cancers cells are present in the organ.

In some embodiments of the method, the cancer cells are lung cancer, breast cancer, prostate cancer, cervical cancer, pancreatic cancer, colon cancer, ovarian cancer, stomach cancer, esophagus cancer, skin cancer, heart cancer, liver cancer, bronchial cancer, testicular cancer, kidney cancer, bladder cancer, spleen cancer, thymus cancer, thyroid cancer, brain cancer, or gall bladder cancer cells.

In some embodiments, the present invention provides a method of reducing one or more symptoms of cancer or of imaging cancer cells. Cancers or cells thereof include, but are not limited to, lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma).

In some embodiments of the method, the compound or composition is detected using a PET imaging device.

In some embodiments, the compound or composition is detected using a SPECT imaging device.

In some embodiments of the above method, the image obtained is a two-dimensional image.

In some embodiments of the above method, the image obtained is a three-dimensional image.

The present invention provides methods relate to the administration of a compound containing an imaging moiety linked to a targeting moiety, i.e. an antibody, peptide or small molecule, that recognizes target proteins or antigens in or on target cells in a subject, and to an albumin-binding moiety, i.e. an antibody, peptide or small molecule, that recognizes albumin.

In some embodiments, the imaging moiety is linked to both a targeting moiety and to an albumin-binding moiety.

In some embodiments, the claimed conjugates are capable of high affinity binding to receptors on cancer cells or other cells to be visualized. The high affinity binding can be inherent to the targeting moiety or the binding affinity can be enhanced by the use of a derivative or fragment of the targeting moiety or by the use of particular chemical linkage between the imaging agent and targeting moiety that is present in the conjugate.

In some embodiments, the claimed conjugates are capable of high affinity binding to receptors on cancer cells or other cells to be visualized and to albumin. The high affinity binding can be inherent to the targeting moiety and to the albumin-binding moiety or the binding affinity can be enhanced by the use of a derivative or fragment of the targeting moiety or by the use of particular chemical linkage between the imaging agent, targeting moiety and/or the albumin-binding moiety that is present in the conjugate.

In some embodiments, the present invention provides a compound having the structure:

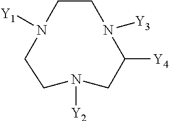

wherein
Y₁ and Y₂ are each, independently, —H, alkylheteroaryl, carboxylic acid, alkyl-carboxylic acid, alkyl-CO₂H, alkylaryl-CO₂H, alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂ or alkylheteroaryl-(NO₂)(P(O)(OH)₂),
   wherein each occurrence of R₁ is, independently, —H, alkyl, alkenyl, alkynyl, alkyl-aryl, alkyl-heteroaryl,
   aryl, heteroaryl, alkyl-CF₃ or —Si(alkyl)₃; and
Y₃ and Y₄ are each, independently, —H, Z₁-L(A), Z₁-L(A)(B), L(A), L(A)(B),

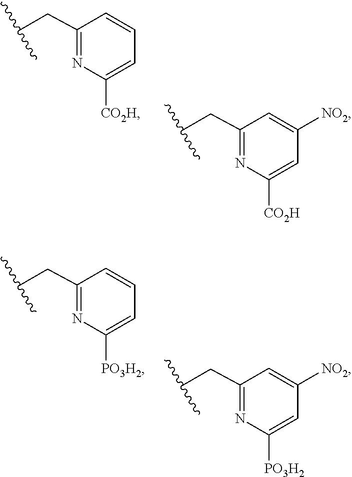

wherein
Z₁ is

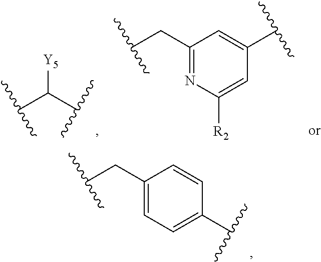

wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
   wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not

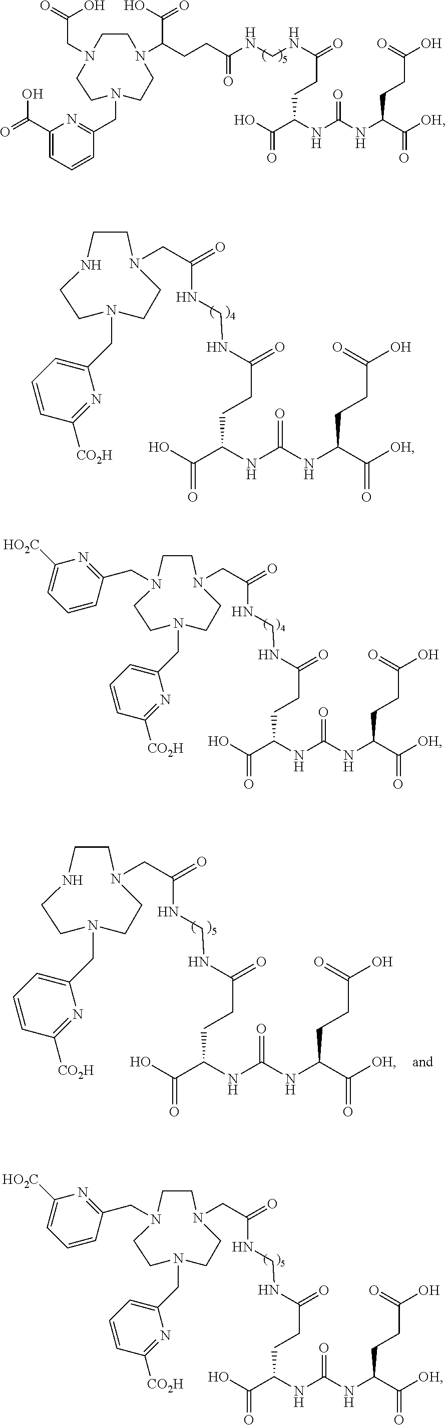

or a pharmaceutically acceptable salt of the compound.

In some embodiments, the present invention provides a compound having the structure:

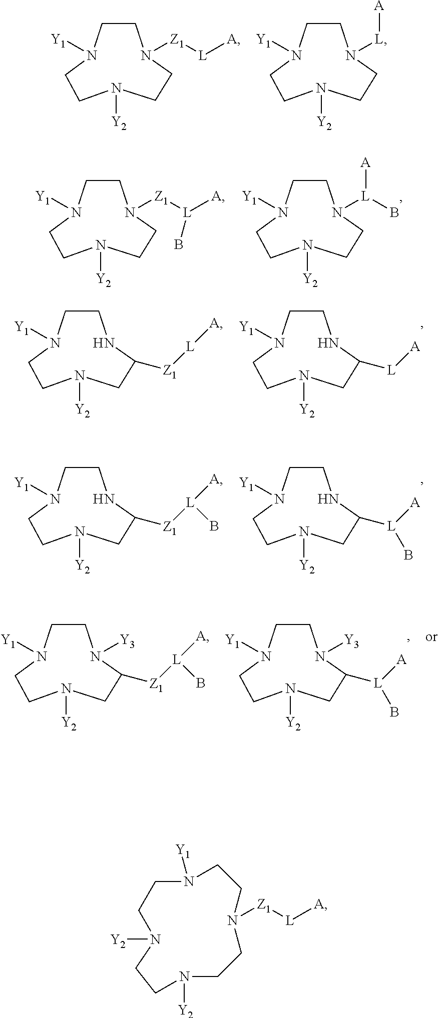

wherein L is a chemical linker, A is a targeting moiety, and B is an albumin-binding moiety.

In some embodiments, Y₁ and Y₂ are each, independently, —H, alkylheteroaryl, carboxylic acid, alkyl-carboxylic acid, alkyl-CO₂H, alkylaryl-CO₂H, alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, or alkyl-N(alkylaryl)₂.

In some embodiments, Y₁ and Y₂ are each, independently, —H, alkylheteroaryl, carboxylic acid, alkyl-carboxylic acid, alkyl-CO₂H, alkylaryl-CO₂H, alkylheteroaryl-CO₂H, or alkylheteroaryl-(NO₂)(CO₂H),

In some embodiments, Y₁ and Y₂ are independently H or carboxylic acid.

In some embodiments, the carboxylic acid is methanoic acid, ethanoic acid, propanoic acid, butanoic acid, pentanoic acid, hexanoic acid, heptanoic acid, octanoic acid, nonanoic acid, or decanoic acid.

In some embodiments, the carboxylic acid is pentanoic acid.

In some embodiments, Y₁ and Y₂ are each, independently, —H, alkylheteroaryl, carboxylic acid, alkyl-carboxylic acid, alkyl-CO₂H, alkylaryl-CO₂H, alkylheteroaryl-CO₂H, alkylheteroaryl-(NO₂)(CO₂H), alkylheteroaryl-P(O)(OH)₂, —P(O)(OH)₂, alkyl-CO₂R₁, alkylaryl-CO₂R₁, alkylheteroaryl-CO₂R₁, alkyl-OH, alkylaryl-OH, alkylheteroaryl-OH, or alkyl-N(alkylaryl)₂.

In some embodiments, Y₁ and Y₂ are each, independently, —H, alkylheteroaryl, carboxylic acid, alkyl-carboxylic acid, alkyl-CO₂H, alkylaryl-CO₂H, alkylheteroaryl-CO₂H, alkylheteroaryl-P(O)(OH)₂, —P(O)(OH)₂, or alkylheteroaryl-(NO₂)(CO₂H).

In some embodiments, Y₁ and Y₂ are independently alkyl-CO₂H, alkylheteroaryl-P(O)(OH)₂, alkylheteroaryl-CO₂H, —P(O)(OH)₂ or carboxylic acid.

In some embodiments, Y₁ or Y₂ is

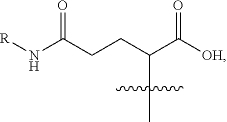

wherein R is H, alkyl, alkenyl, alkynyl, alkyl-aryl, alkyl-heteroaryl, aryl, heteroaryl, alkyl-CF₃ or —Si(alkyl)₃.

In some embodiments, the present invention provides a compound having the structure:

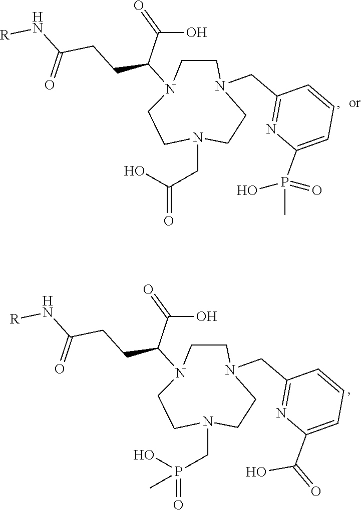

wherein R is H, alkyl, alkenyl, alkynyl, alkyl-aryl, alkyl-heteroaryl, aryl, heteroaryl, alkyl-CF₃ or —Si(alkyl)₃.

In some embodiments, the present invention provides a compound having the structure:

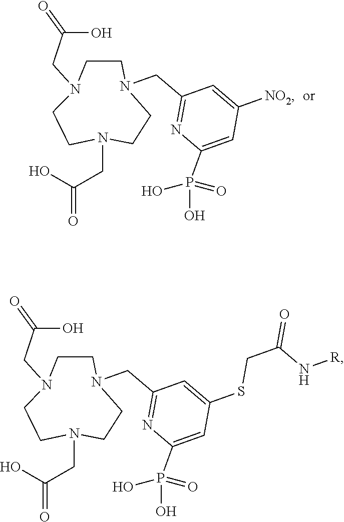

wherein R is H, alkyl, alkenyl, alkynyl, alkyl-aryl, alkyl-heteroaryl, aryl, heteroaryl, alkyl-CF₃ or —Si(alkyl)₃.

In some embodiments, the present invention provides a compound having the structure:

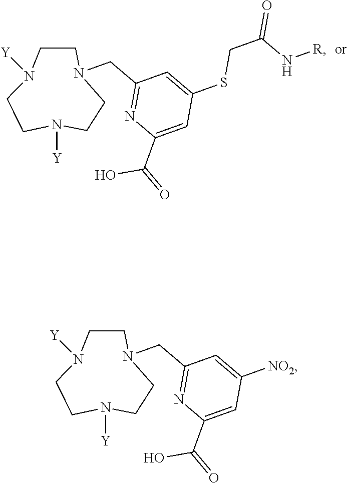

wherein R is H, alkyl, alkenyl, alkynyl, alkyl-aryl, alkyl-heteroaryl, aryl, heteroaryl, alkyl-CF₃ or —Si(alkyl)₃.

In some embodiments, A is ((5-(2-(4-(aminomethyl)cyclohexane-1-carboxamido)-3-(naphthalen-2-yl)propanamido)-1-carboxypentyl)carbamoyl)glutamic acid or a derivative or fragment thereof; 2-[3-(1,3-dicarboxypropyl)ureido]pentanedioic acid (DUPA) or a derivative or fragment thereof; trastuzumab, bombesin or somatostatin or a derivative or fragment thereof.

In some embodiments, the albumin-binding moiety B is a small molecule, a peptide, a protein or an antibody or a derivative or fragment thereof, more preferably, the albumin-binding moiety B is 4-(4-iodophenyl)butanoic acid or a derivative or fragment thereof, or the albumin-binding moiety B is 4-(4-methyl)butanoic acid or a derivative or fragment thereof.

In some embodiments, the present invention provides a metal complex comprising the compound disclosed in this application.

In some embodiments, the metal in the metal complex is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb).

In some embodiments, the metal-ion in the metal complex is Scandium-Fluorine-18 (^{nat}Sc-¹⁸F), Lanthanum-Fluorine-18 (^{nat}La-¹⁸F), or Lutetium-Fluorine-18 (^{nat}Lu-¹⁸F).

In some embodiments, the present invention provides a compound having the structure:

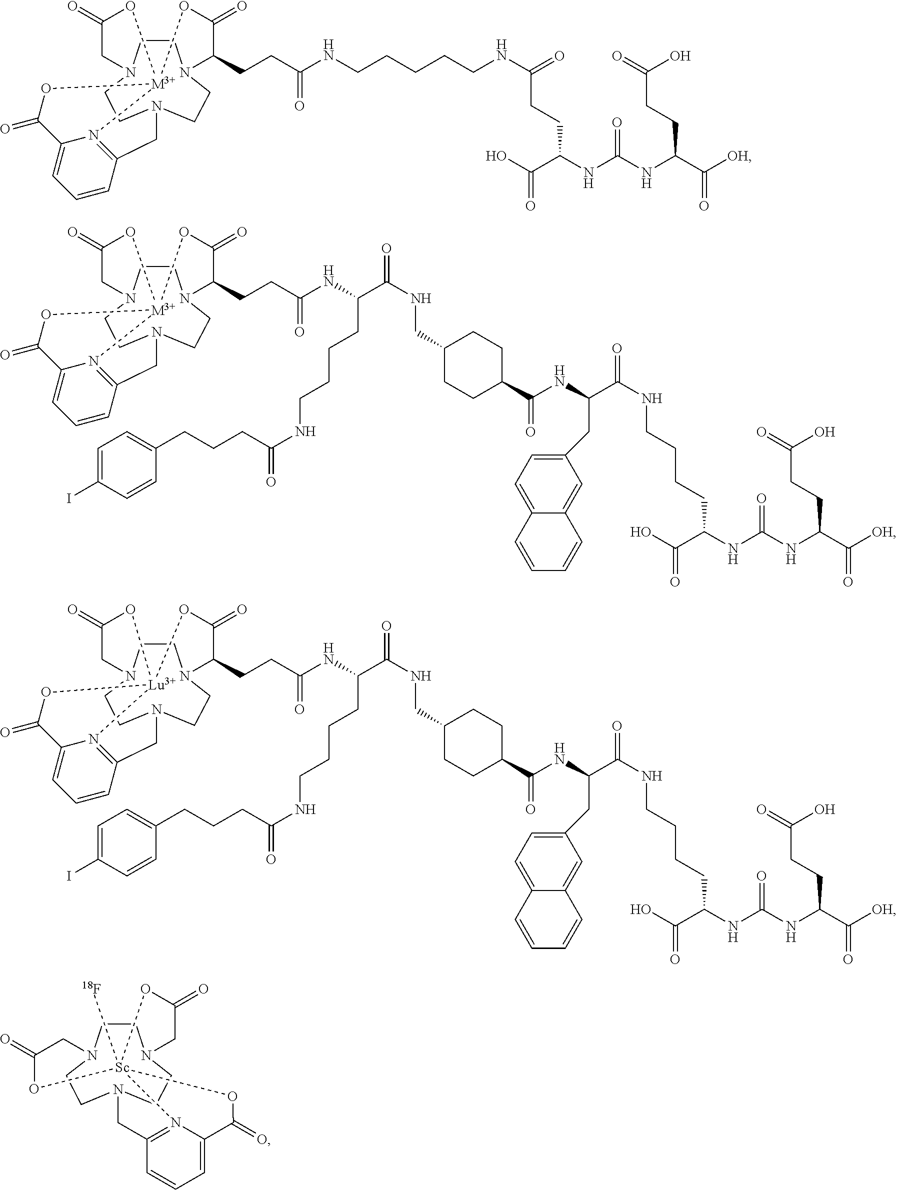

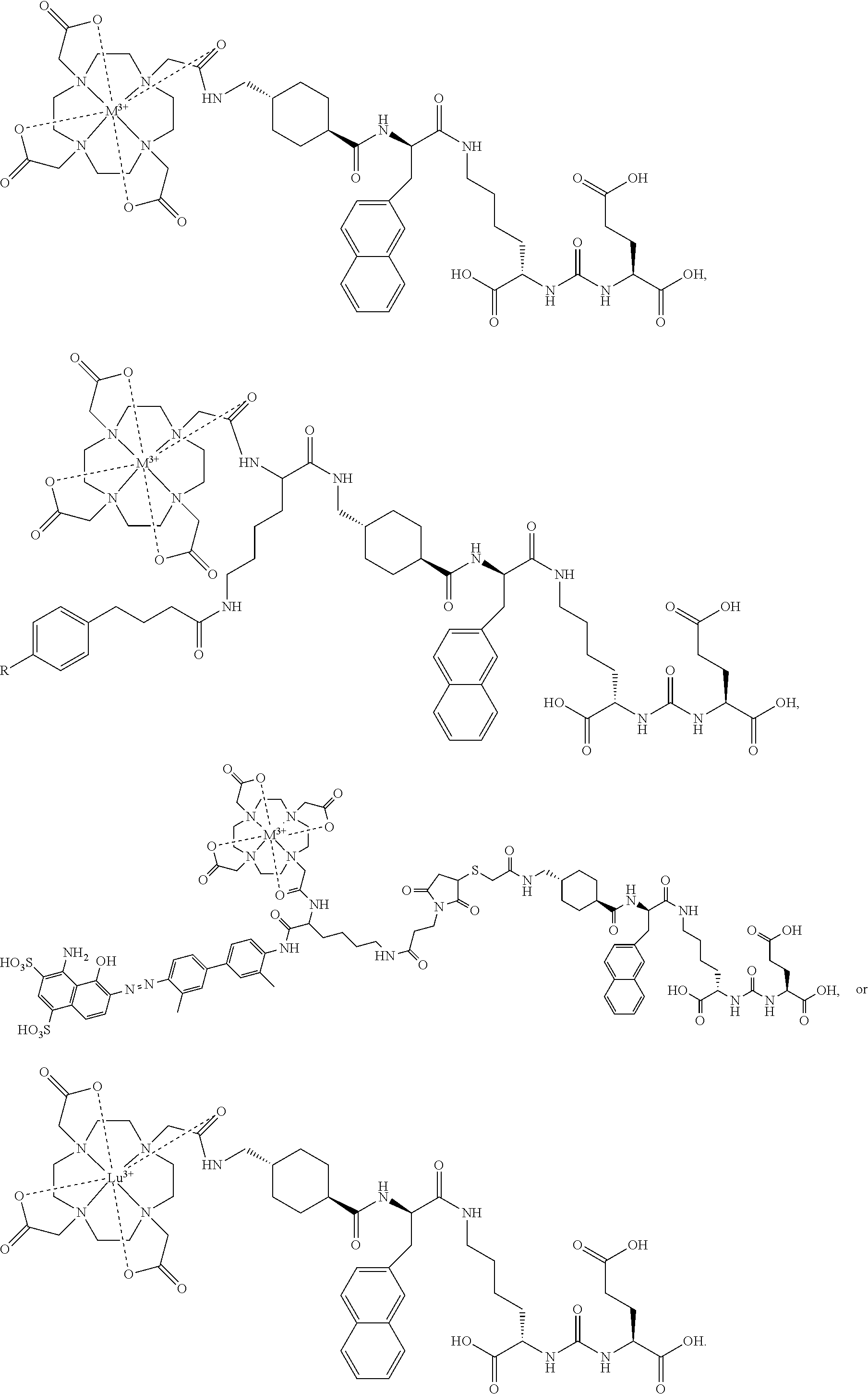

In some embodiments, the present invention provides a pharmaceutical composition comprising the compound of or the metal complex disclosed in this application, and a pharmaceutically acceptable carrier.

In some embodiments, the present invention provides a method of detecting target cells in a subject comprising administering an effective amount of the metal complex or the composition disclosed in this application to the subject, and imaging the subject with a molecular imaging device to detect the metal complex or composition in the subject.

In some embodiments, the present invention provides a method of imaging target cells in a subject comprising:
1) administering to the subject an effective amount of the metal complex or the composition disclosed in this application or a pharmaceutically acceptable salt thereof, wherein the compound specifically accumulates at the target cells in the subject;
2) detecting in the subject the location of the metal complex or the composition; and
3) obtaining an image of the target cells in the subject based on the location of the metal complex or the composition in the subject.

In some embodiments, the present invention provides a method of detecting the presence of target cells in a subject which comprises determining if an amount of the metal complex or a pharmaceutically acceptable salt thereof, or the composition disclosed in this application is present in the subject at a period of time after administration of the metal complex or composition to the subject, thereby detecting the presence of the target cells based on the amount of the metal complex or composition determined to be present in the subject.

In some embodiments, the detecting and imaging is performed by a Positron Emission Tomography (PET) device or a Single-Photon Emission Computed Tomography (SPECT) device.

In some embodiments, the target cells are cancer cells; preferably, the cancer cells are prostate cancer cells, more preferably, the cancer cells have elevated levels of prostate-specific membrane antigen (PSMA).

In some embodiments, the present invention provides a method of reducing the size of a prostate tumor or of inhibiting proliferation of prostate cancer cells comprising contacting the tumor or cancer cells with the metal complex or a pharmaceutically acceptable salt thereof, or the composition disclosed in this application, so as to thereby reducing the size of the tumor or inhibit proliferation of the cancer cells.

### Imaging Agent

As used herein, the term “imaging agent” refers to any agent or portion (i.e. imaging moiety) of an agent that is used in medical imaging to visualize or enhance the visualization of the body including, but not limited to, internal organs, cells, cancer cells, cellular processes, tumors, and/or normal tissue. Imaging agents or imaging moieties include, but are not limited to, PET imaging agents, SPECT imaging agents. Imaging agents or moieties include, but are not limited to, any compositions useful for imaging cancer cells.

The imaging moiety of the compound of the present invention has the structure:

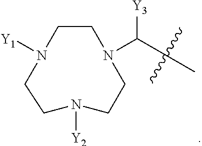

### Targeting Agent

The targeting moiety may comprise, consist of, or consist essentially of an antibody, peptide, protein or small molecule.

The targeting moiety may comprise, consist of, or consist essentially of Brentuximab (targets cell-membrane protein CD30), Inotuzumab targets CD22), Gemtuzumab (targets CD33), Milatuzumab (targets CD74), Trastuzumab (targets HER2 receptor), Glembatumomab (targets transmembrane glycoprotein NMB-GPNMB), Lorvotuzumab (targets CD56), or Labestuzumab (targets carcinoembryonic cell adhesion molecule 5) or derivatives or fragments thereof.

The targeting moiety may comprise, consist of, or consist essentially of ((5-(2-(4-(aminomethyl)cyclohexane-1-carboxamido)-3-(naphthalen-2-yl)propanamido)-1-carboxypentyl)carbamoyl)glutamic acid (targets prostate-specific membrane antigen (PSMA)), or derivatives or fragments thereof.

The targeting moiety may comprise, consist of, or consist essentially of (((S)-5-((R)-2-((1r,4R)-4-(aminomethyl)cyclohexane-1-carboxamido)-3-(naphthalen-2-yl)propanamido)-1-carboxypentyl)carbamoyl)-L-glutamic (targets prostate-specific membrane antigen (PSMA)), or derivatives or fragments thereof.

The targeting moiety may comprise, consist of, or consist essentially of DUPA [(2-[3-(1, 3-dicarboxy propyl)ureido]pentanedioic acid)] (targets prostate-specific membrane antigen (PSMA)), or derivatives or fragments thereof.

The targeting moiety may comprise, consist of, or consist essentially of bombesin (targets G-protein-coupled receptors BBR1, -2, and -3) or somatostatin (targets Somatostatin receptor subtypes 1-5), or derivatives or fragments thereof.

The targeting moiety is capable of selectively binding to the population of cells to be visualized due to preferential expression on the targeted cells of a receptor for the targeting moiety. The binding site for the targeting moiety can include receptors or other proteins that are uniquely expressed, overexpressed, or preferentially expressed by the population of cells to be visualized. A surface-presented protein uniquely expressed, overexpressed, or preferentially expressed by the cells to be visualized is a receptor not present or present at lower amounts on other cells providing a means for selective, rapid, and sensitive visualization of the cells targeted for diagnostic imaging using the conjugates of the present invention.

### ((5-(2-(4-(aminomethyl)cyclohexane-1-carboxamido)-3-(naphthalen-2-yl)propanamido)-1-carboxypentyl)carbamoyl)glutamic acid has the Structure

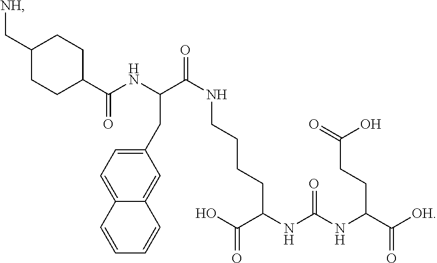

### (((S)-5-((R)-2-((1r,4R)-4-(aminomethyl)cyclohexane-1-carboxamido)-3-(naphthalen-2-yl)propanamido)-1-carboxypentyl)carbamoyl)-L-glutamic has the Structure

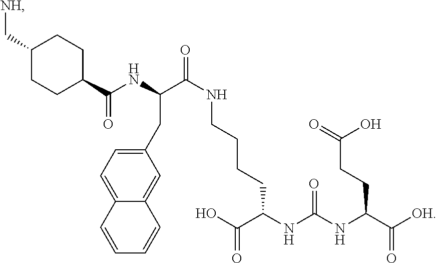

### 2-[3-(1,3-dicarboxypropyl)ureido]pentanedioic acid (DUPA) has the Structure

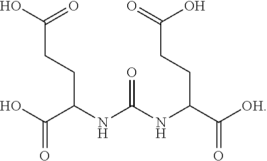 [SMILES: O=C(O)CCC(NC(=O)NC(CCC(=O)O)C(=O)O)C(=O)O]

Exemplary targeting moieties are described in U.S. Pat. Nos. 10,005,820 B2, 9,801,951 B2 or U.S. Patent Application Publication No. 2015/0105540 A1, the contents of which are hereby incorporated by reference.

### Albumin-Binding Agent

The albumin-binding moiety may comprise, consist of, or consist essentially of an antibody, peptide, protein or small molecule.

The albumin-binding moiety may comprise, consist of, or consist essentially of 4-(4-iodophenyl)butanoic or 4-(4-methyl)butanoic acid or derivatives or fragments thereof.

The albumin-binding moiety is capable of selectively binding to plasma proteins such as human serum albumin (HSA).

### Chemical Linker

The term “chemical linker” or “linker” refers to a chemical moiety or bond that covalently attaches two or more molecules, such as an imaging moiety, a targeting moiety and an albumin-binding moiety. The linker may be a cleavable linker, e.g. pH-sensitive (acid-labile) linker, disulfide linker, a peptide linker, a β-glucuronide linkers or a hydrazine linker. The linker may be a non-cleavable linker, e.g. thioether, maleimidocaproyl, maleimidomethyl cyclohexane-carboxylate, alkyl, alkylamido or amide linker.

Covalent bonding of the imaging agent and chemical linker to both the targeting moiety and albumin-binding moiety can occur through the formation of amide, ester or imino bonds between acid, aldehyde, hydroxy, amino, or hydrazo groups. For example, a carboxylic acid on the targeting moiety can be activated using carbonyldiimidazole or standard carbodiimide coupling reagents such as 1-ethyl-3-(3-dimethylaminopropyl)-carbodiimide (EDC) and thereafter reacted with the other component of the conjugate, or with a linker, having at least one nucleophilic group, i.e. hydroxy, amino, hydrazo, or thiol, to form the vitamin-chelator conjugate coupled, with or without a linker, through ester, amide, or thioester bonds.

Linkage of a targeting moiety and albumin-binding moiety to the imaging moiety may be achieved by any means known to those in the art, such as genetic fusion, covalent chemical attachment, noncovalent attachment (e.g., adsorption) or a combination of such means. Selection of a method for linking a targeting moiety and an albumin-binding moiety to an imaging moiety will vary depending, in part, on the chemical nature of the targeting moiety and the albumin-binding moiety.

Linkage may be achieved by covalent attachment, using any of a variety of appropriate methods. For example, the targeting moiety, albumin-binding moiety and imaging moiety may be linked using trifunctional reagents (linkers) that are capable of reacting with each of the targeting moiety, albumin-binding moiety and imaging moiety and forming a bridge between the three.

The term “non-covalent linker” is used in accordance with its ordinary meaning and refers to a divalent or trivalent moiety which includes at least two molecules that are not covalently linked to each other but do interact with each other via a non-covalent bond (e.g. electrostatic interactions (e.g. ionic bond, hydrogen bond, halogen bond) or van der Waals interactions (e.g. dipole-dipole, dipole-induced dipole, London dispersion).

The terms “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), hydrazine (N₂H₄)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation).

A photocleavable linker (e.g., including or consisting of a o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of an reducing agent (e.g., Tris(3-hydroxypropyl)phosphine). In embodiments, the cleavable linker is a dialkylketal linker, an azo linker, an allyl linker, a cyanoethyl linker, a 1-(4,4-dimethyl-2,6-dioxocyclohex-1-ylidene)ethyl linker, or a nitrobenzyl linker.

The term “orthogonally cleavable linker” or “orthogonal cleavable linker” as used herein refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent. In embodiments, an orthogonally is a cleavable linker that following cleavage the two separated entities (e.g., fluorescent dye, bioconjugate reactive group) do not further react and form a new orthogonally cleavable linker.

Exemplary linkers are described in U.S. Patent Application No. 2012/0322741 A1, U.S. Patent Application No. 2018/0289828 A1 and U.S. Pat. No. 8,461,117 B2, the contents of which are hereby incorporated by reference.

### Antibody

An “antibody” as used herein is defined broadly as a protein that characteristically immunoreacts with an epitope (antigenic determinant) of an antigen. As is known in the art, the basic structural unit of an antibody is composed of two identical heavy chains and two identical light chains, in which each heavy and light chain consists of amino terminal variable regions and carboxy terminal constant regions. The antibodies of the present invention include polyclonal antibodies, monoclonal antibodies (mAbs), chimeric antibodies, CDR-grafted antibodies, humanized antibodies, human antibodies, catalytic antibodies, multispecific antibodies, as well as fragments, regions or derivatives thereof provided by known techniques, including, for example, enzymatic cleavage, peptide synthesis or recombinant techniques.

As used herein, “monoclonal antibody” means an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants, each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495-97 (1975), or may be made by recombinant DNA methods (see, e.g., U.S. Pat. No. 4,816,567). The monoclonal antibodies may also be isolated from phage display libraries using the techniques described, for example, in Clackson et al., Nature 352:624-28 (1991) and Marks et al., J. Mol. Biol. 222 (3): 581-97 (1991).

The term “hybridoma” or “hybridoma cell line” refers to a cell line derived by cell fusion, or somatic cell hybridization, between a normal lymphocyte and an immortalized lymphocyte tumor line. In particular, B cell hybridomas are created by fusion of normal B cells of defined antigen specificity with a myeloma cell line, to yield immortal cell lines that produce monoclonal antibodies. In general, techniques for producing human B cell hybridomas, are well known in the art [Kozbor et al., Immunol. Today 4:72 (1983); Cole et al., in Monoclonal Antibodies and Cancer Therapy, Alan R. Liss, Inc. 77-96 (1985)].

The term “epitope” refers to a portion of a molecule (the antigen) that is capable of being bound by a binding agent, e.g., an antibody, at one or more of the binding agent's antigen binding regions. Epitopes usually consist of specific three-dimensional structural characteristics, as well as specific charge characteristics.

“Humanized antibodies” means antibodies that contain minimal sequence derived from non-human immunoglobulin sequences. For the most part, humanized antibodies are human immunoglobulins (recipient antibody) in which residues from a hyper variable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues (see, for example, U.S. Pat. Nos. 5,585,089; 5,693,761; 5,693,762, each herein incorporated by reference). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-25 (1986); Riechmann et al., Nature 332:323-27 (1988); and Presta, Curro Opin. Struct. Biol. 2:593-96 (1992), each of which is incorporated herein by reference.

Also encompassed by the term “antibody” are xenogeneic or modified antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598, the entire contents of which are incorporated herein by reference.

Those skilled in the art will be aware of how to produce antibody molecules of the present invention. For example, polyclonal antisera or monoclonal antibodies can be made using standard methods. To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art. Hybridoma cells can be screened immunochemically for production of antibodies which are specifically reactive with the oligopeptide, and monoclonal antibodies isolated.

### Target Cells

The term “target cells” refers to the cells that are involved in a pathology and so are preferred targets for imaging or therapeutic activity. Target cells can be, for example and without limitation, one or more of the cells of the following groups: primary or secondary tumor cells (the metastases), stromal cells of primary or secondary tumors, neoangiogenic endothelial cells of tumors or tumor metastases, macrophages, monocytes, polymorphonuclear leukocytes and lymphocytes, and polynuclear agents infiltrating the tumors and the tumor metastases. The term “targeting moiety” and “targeting agent” refer to an antibody, aptamer, peptide, small molecule or other substance that binds specifically to a target. A targeting moiety may be an antibody targeting moiety (e.g. antibodies or fragments thereof) or a non-antibody targeting moiety (e.g. aptamers, peptides, small molecules or other substances that bind specifically to a target).

The term “target tissue” refers to target cells (e.g., tumor cells) and cells in the environment of the target cells.

The term “cancer” refers to any of a number of diseases characterized by uncontrolled, abnormal proliferation of cells, the ability of affected cells to spread locally or through the bloodstream and lymphatic system to other parts of the body (e.g., metastasize), as well as any of a number of characteristic structural and/or molecular features. A “cancerous cell” or “cancer cell” is understood as a cell having specific structural properties, which can lack differentiation and be capable of invasion and metastasis. Examples of cancers are, breast, lung, brain, bone, liver, kidney, colon, and prostate cancer.

Exemplary targets are described in Avicenna J Med Biotechnol. 2019 January-March; 11 (1): 3-23, Nature Reviews Drug Discovery Volume 16, pages 315-337 (2017), the contents of which are hereby incorporated by reference.

### Other Definitions

As used herein, the term “amino acid” refers to any natural or unnatural amino acid including its salt form, ester derivative, protected amine derivative and/or its isomeric forms. Amino Acids comprise, by way of non-limiting example: Agmatine, Alanine Beta-Alanine, Arginine, Asparagine, Aspartic Acid, Cysteine, Glutamine, Glutamic Acid, Glycine, Histidine, Isoleucine, Leucine, Lysine, Methionine, Phenylalanine, Phenyl Beta-Alanine, Proline, Serine, Threonine, Tryptophan, Tyrosine, and Valine. The amino acids may be L or D amino acids.

The terms “peptide”, “polypeptide”, peptidomimetic and “protein” are used to refer to a polymer of amino acid residues. The terms apply to amino acid polymers in which one or more amino acid residues is an artificial chemical mimetic of a corresponding naturally occurring amino acid, as well as to naturally occurring amino acid polymers. These terms also encompass the term “antibody”. “Peptide” is often used to refer to polymers of fewer amino acid residues than “polypeptides” or “proteins”. A protein can contain two or more polypeptides, which may be the same or different from one another.

As used herein, the term “oligopeptide” refers to a peptide comprising of between 2 and 20 amino acids and includes dipeptides, tripeptides, tetrapeptides, pentapeptides, etc.

An amino acid or oligopeptide may be covalently bonded to an amine of another molecule through an amide linkage, resulting in the loss of an “OH” from the amino acid or oligopeptide.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure result.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, isoxazoline, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, acridinyl, carbazolyl, quinolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(CH₄N) and the like.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As sued herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons and five hydrogens.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5th Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, the present invention provides a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30th edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric coated to prevent release of the active ingredient compounds before they reach the small intestine. Materials that may be used as enteric coatings include, but are not limited to, sugars, fatty acids, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

The compounds of the present invention can be synthesized according to general Schemes. Variations on the following general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

While the invention has been shown and described with reference to certain embodiments of the present invention thereof, it will be understood by those skilled in the art that various changes in from and details may be made therein without departing from the spirit and scope of the present invention and equivalents thereof.

### Experimental Details

### Materials and Methods

All starting materials were purchased from Acros Organics, Alfa Aesar, Macrocyclics, Sigma Aldrich, or TCI America and used without further purification. Mass spectrometry: low-resolution electrospray ionization (ESI) mass spectrometry was carried out on an Agilent 1260 Infinity II HPLC with a G6125B single quadrupole mass detector. High-resolution (ESI) mass spectrometry was carried out at the Stony Brook University Center for Advanced Study of Drug Action (CASDA) mass spectrometry facility with an Agilent LC-UV-TOF spectrometers. Inductively coupled plasma spectroscopy (ICP) was performed on an Agilent Technologies ICP-OES (Model 5110). UV-VIS spectra were collected with the NanoDrop 1C instrument (AZY1706045). Spectra were recorded from 190 to 850 nm in a quartz cuvette with 1 cm path length. HPLC: Preparative HPLC was carried out using a Shimadzu HPLC-20AR equipped with a Binary Gradient, pump, UV-Vis detector, manual injector on a Phenomenex Luna C18 column (250 mm×21.2 mm, 100 A, AXIA packed). Method A (preparative purification method): A=0.1% TFA in water, B=0.1% TFA in MeCN. Gradient: 0-5 min: 5% B. 5-24 min: 5-95% B. RadioHPLC analysis was carried out using a Shimadzu HPLC-20AR equipped with a binary gradient, pump, UV-Vis detector, autoinjector and Laura radiodetector on a Phenomenex Luna C18 column (150 mm×3 mm, 100 A). Method B: A=0.1% TFA in water, B=0.1% TFA in MeCN with a flow rate of 0.8 mL/min, UV detection at 220 and 254 nm. Gradient 0-1 min: 5% B. 1-16 min 5-95% B. Compounds 1, PSMA-617, picaga tris t-butyl ester, and Lu-mpaten were synthesized according to previously published procedures. ¹⁷⁷Lu was obtained from the DOE isotope program, produced at the University of Missouri reactor.

### Example 1. Complexation with ⁴⁴Sc

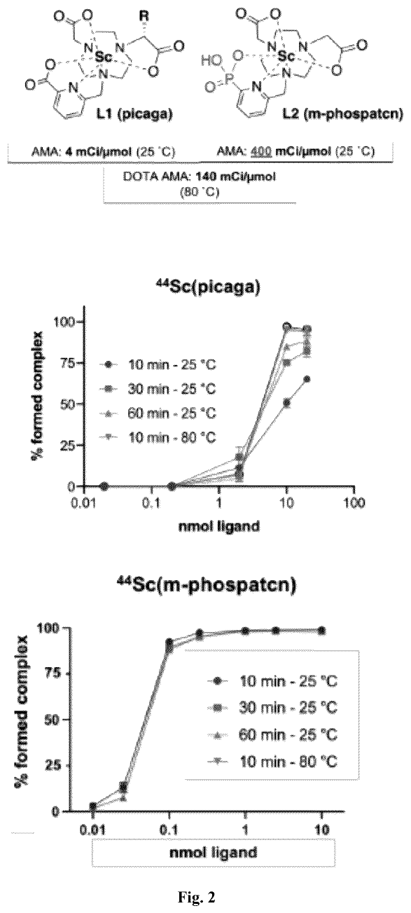

Picaga derivatives described herein are improved Sc chelators suitable for kit formulations. To be compatible with clinical radiopharmacies these formulations must exhibit accelerated room temperature complexation kinetics and enhanced apparent molar activity of ideally >0.1 Ci/μmol. The azamacrocycle-based chelator L2/m-phospaten is a phosphonate-containing chelator characterized by enhanced inner sphere crowding and a first deprotonation event occurring at lower pH which accelerates kₒₙ for lanthanides and Cu(II) (Anderson, C. J. & Ferdani, R. 2009). A drastic improvement of the achievable apparent molar activity (AMA) with ⁴⁴Sc: at room temperature, AMA was improved to 0.4 Ci/μmol, which corresponds to an improvement of two orders of magnitude when compared to picaga (0.004 Ci/μmol) (Vaughn, B. A. et al. 2020) and a greater than 2-fold improvement compared to the AMA of DOTA at 80° C. (0.14 Ci/μmol) in comparative experiments (FIG. 2). Accelerated kₒₙ for m-phospaten with complexations reaching equilibria within <10 minutes whereas picaga required ˜60 min to achieve equilibrium at room temperature is also shown (FIG. 2). Fast kₒₙ and a high AMA are essential for a bifunctional chelator compatible with kit formulations akin to 68Ga-PSMA-11, where a typical AMA's for clinical tracer synthesis corresponds to 1 Ci/μmol (Fuscaldi, L. L. et al. 2021).

### Example 2. Complexation with ¹⁷⁷Lu

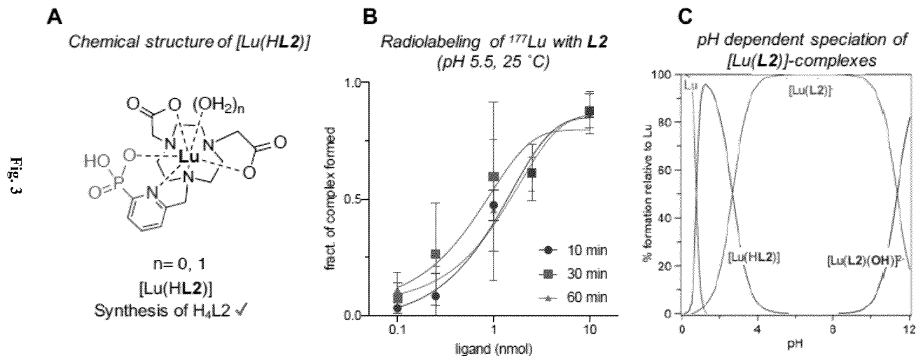

Corresponding radiolabeling experiments with Lu-177 have revealed that L2 is indeed capable of chelating Lu-177 at room temperature. The concentration versus radiochemical yield plot in the dependence of reaction time is provided in FIG. 3B, demonstrating efficient room temperature radiolabeling at 10 nmol ligand quantities. Furthermore, spectrophotometric pH-dependent titration of the [Lu(L2)] complex shows a pronounced shift of the complex formation pKa to pH 0.9. This results in marked improvement of complexation properties with respect to kinetics and low pH complexation properties (FIG. 3C), when compared to the first-generation chelator L1. As was previously shown, radiolabeling of Lu-177 with L1 is not feasible at room temperature, in part due to the comparatively high formation pKa of the [Lu(L1)] coordination complex (pH 2.4) (Vaughn, B. A. et al. 2021). These results show that phosphonate functionalized analogues such as L2 can efficiently complex Lu-177 at room temperature.

### Example 3. Complexation with Sc-18F

The diagnostic, positron-emitting isotope fluorine-18 is widely produced on a clinical scale. Sc-18F/¹⁷⁷Lu represents a potential clinically viable theranostic pair. The development of Sc-18F radiochelation chemistry has the potential to mitigate long synthesis and purification protocols associated with covalent ¹⁸F radiolabeling.

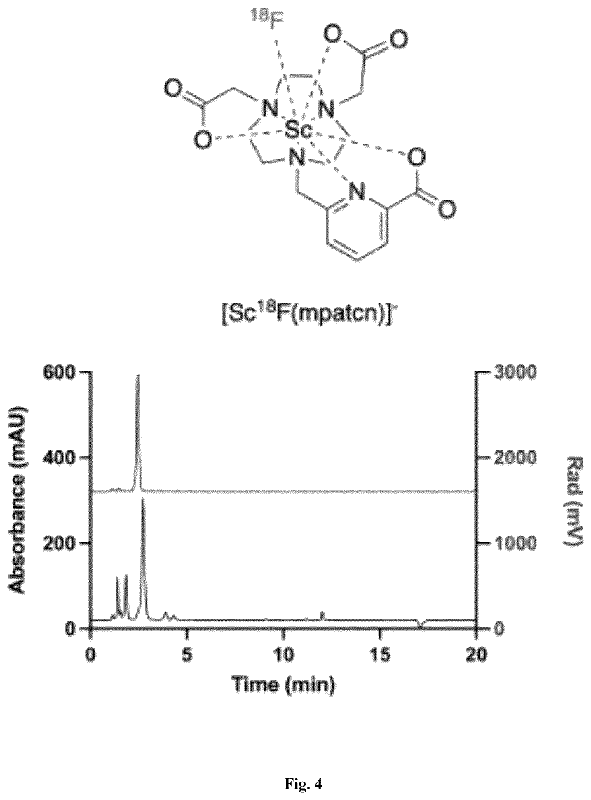

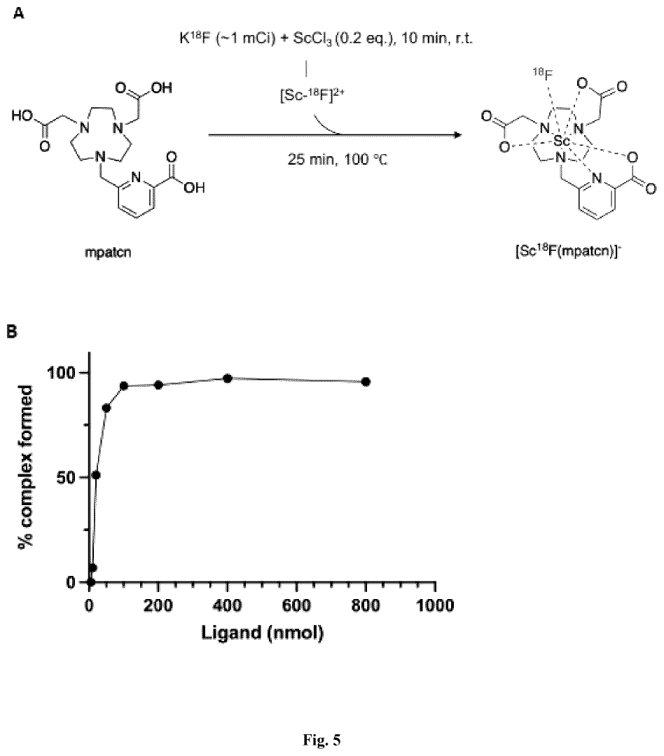

Initial studies identified mpaten as a lead ligand for complexation with Sc-18F (FIG. 4A). Pre-formed Sc-¹⁸F complex (˜1 mCi 18F, 0.2 eq. ScCl₃) was incubated at 100° C., pH 4 with 800 nmol ligand (FIG. 4A). Subsequently, radiolabeling optimization was achieved with 94% radiochemical yield (RCY) with 100 nmol mpatcn (FIG. 5A, B). The Sc-¹⁸F complex [Sc-¹⁸F]²⁺ was first prepared (K18F (˜1 mCi)+ScCl₃ (0.2 eq.), 10 min, r.t.), then the preformed complex was incubated with 100 nmol mpaten to produce [Sc¹⁸F(mpatcn)]⁻ in 94% RCY (FIG. 5A, B). FIG. 5B also shows the dependence of complex formation on ligand concentration.

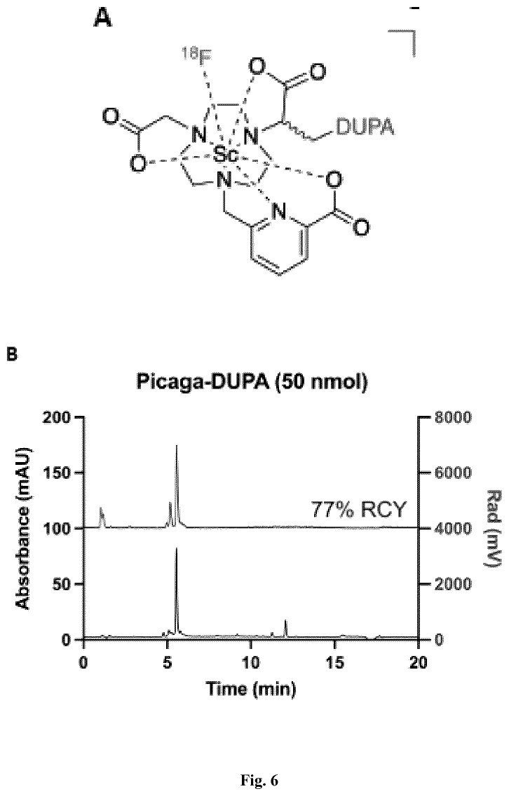

Complexation of picaga-DUPA conjugate with preformed Sc-¹⁸F complex was also achieved to form [Sc-¹⁸F(picaga-DUPA)]⁻ (FIG. 6A) in high radiochemical yield (77%) (FIG. 6B). Pre-formed Sc-¹⁸F complex (˜1 mCi 18F, 0.2 eq. ScCl₃) was incubated with 50 nmol ligand at 100° C., pH 4.

### Example 4. Formulation Stability of [Sc-¹⁸F (Picaga-DUPA)]

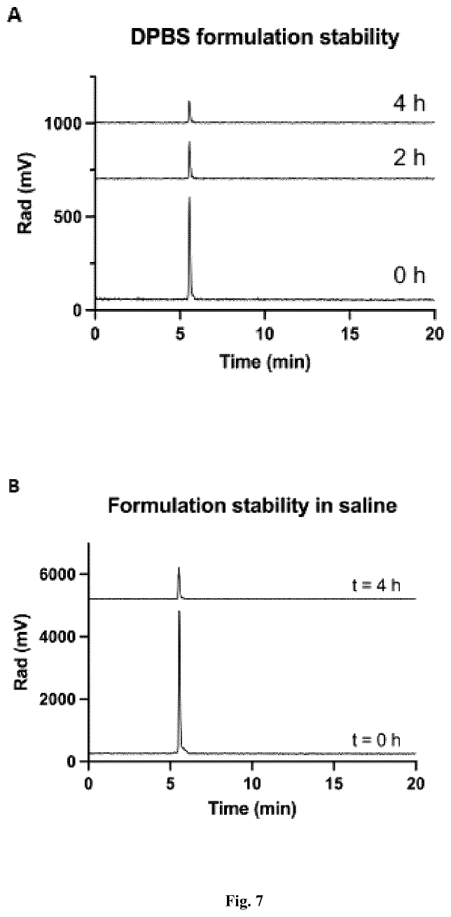

The stability of [Sc-¹⁸F(picaga-DUPA)]⁻ in both DPBS and saline was evaluated for up to 4 h. It was determined that [Sc-¹⁸F(picaga-DUPA)]⁻ was stable in both DPBS and saline for at least 4 h (FIG. 7A, B).

### Example 5. Biodistribution in Mice Models of Prostate Cancer

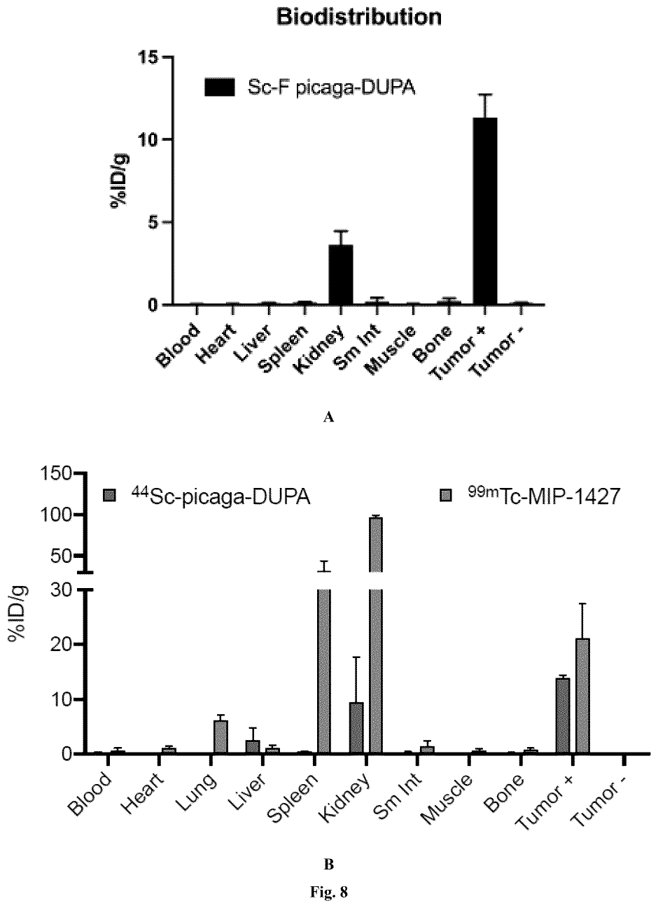

To assess the in vivo behavior of [Sc-¹⁸F(picaga-DUPA)], the radiolabeled compound was administered to mice bearing PSMA+ and PSMA-tumor xenografts on the right and the left flank respectively. The results show that [Sc-¹⁸F(picaga-DUPA)]⁻ exhibits high PSMA+tumor uptake, high in vivo stability and favorable biodistribution in normal tissues (FIG. 8).

### Example 6. Synthetic Schemes

### I. Synthesis of L2

### 1. General Methods

Preparative HPLC was carried out on a Phenomenex Luna C18 column (250 mm×21.2 mm, 100 Å, AXIA packed) at a flow rate of 15 mL/min using a Shimadzu HPLC-20AR equipped with a binary gradient pump, UV-vis detector, and manual injector. UV absorption was recorded at 254 nm.

Method A: Gradient: 0-1 min: 5% B; 1-14 min: 5-50% B; 14-23 min: 50-95% B; 23-26 min: 95% B; 26-27 min: 95-5% B; 27-30 min: 5% B.

### 2. Synthetic Scheme

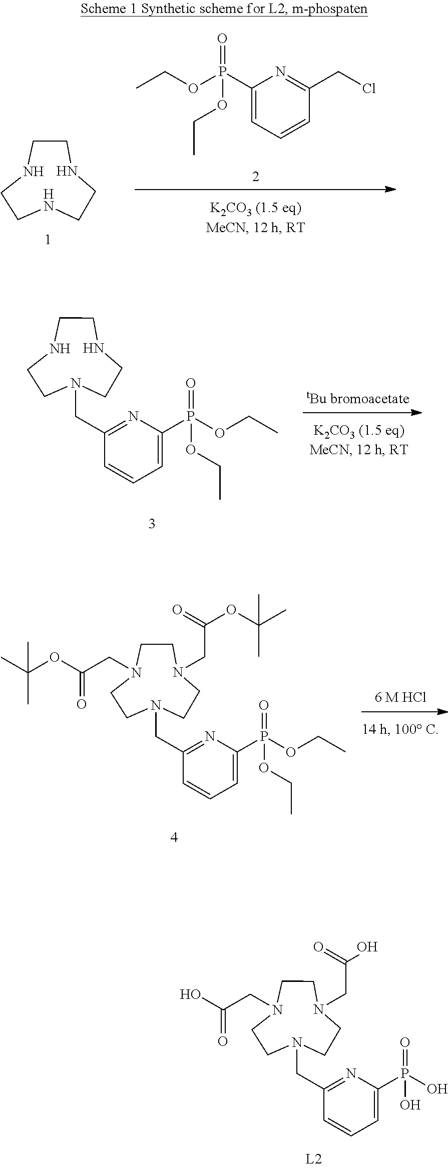 [SMILES: C1CNCCNCCN1 ; CCOP(=O)(OCC)c1cccc(CCl)n1 ; CCOP(=O)(OCC)c1cccc(CN2CCNCCNCC2)n1 ; CCOP(=O)(OCC)c1cccc(CN2CCN(CC(=O)OC(C)(C)C)CCN(CC(=O)OC(C)(C)C)CC2)n1 ; O=C(O)CN1CCN(CC(=O)O)CCN(Cc2cccc(P(=O)(O)O)n2)CC1]

Diethyl (6-(chloromethyl)pyridine-2-yl)phosphonate (2) was synthesized in two steps according to a previously published procedure) (Salaam et al. 2018).

### Diethyl 6-[(1,4,7-triazonan-1-yl)methyl]-2-pyridinephosphonate (3)

A suspension of 1,4,7-triazacyclononane (1) (140.7 mg, 1.091 mmol), compound 2 (286.3 mg, 1.089 mmol) and K₂CO₃ (224.7 mg, 1.628 mmol) in acetonitrile (3 mL) was stirred at room temperature for 12 hours. The reaction mixture was filtered and the filtrated was concentrated in vacuo. The crude mixture was purified with reverse phase preparative HPLC (Method A) with the product eluting at 11.6 min. The fractions containing product were combined and the solvent was removed in vacuo to afford 3 as an amber oil (102.3 mg, 26% yield).

### Diethyl 6-{[4,7-bis(tert-butoxycarbonylmethyl)-1,4,7-triazonan-1-yl]methyl}-2-pyridinephosphonate (4)

A suspension of 3 (102.3 mg, 0.2872 mmol), tert-butyl bromoacetate (128 μL, 0.817 mmol) and K₂CO₃ (200.7 mg, 1.454 mmol) in acetonitrile (7.5 mL) was stirred at room temperature for 12 hours. The reaction mixture was filtered and the filtrated was concentrated in vacuo. The resulting mixture was purified with reverse phase preparative HPLC (Method A) with the product eluting at 19.2 min. The fractions containing product were combined and the solvent was removed in vacuo to afford 4 as a yellow oil (49.3 mg, 29% yield).

6-{[4,7-Bis(carboxymethyl)-1,4,7-triazonan-1-yl]methyl}-2-pyridinephosphonate (L2), mphospatcn Compound 4 (25.5 mg, 0.0436 mmol) was dissolved in 6 M HCl (2 mL) and refluxed for 14 hours. The solvent was removed under vacuum to afford L2 as an off-white solid in HCl salt (14.7 mg, 81%) without further purification.

### II. Synthesis of L2-NO₂. (S)-2,2′-(2-(4-nitrobenzyl)-7-((6-phosphonopyridin-2-yl)methyl)-1,4,7-triazonane-1,4-diyl)diacetic acid

### 1. Experimental Procedures

All HPLC purification and analytical methods were conducted using a binary solvent system in which solvent A was water+0.1% TFA and solvent B was MeCN+0.1% TFA. Preparative HPLC was carried out using a Shimadzu HPLC-20AR equipped with a binary gradient pump, UV-vis detector, and manual injector. UV absorption was recorded at 254 nm.

Method A: Phenomenex Luna C18 column (250 mm×21.2 mm, 100 Å, AXIA packed) at a flow rate of 15 mL/min. Gradient: 0-1 min: 5% B; 1-14 min: 5-50% B; 14-23 min: 50-95% B; 23-26 min: 95% B; 26-27 min: 95-5% B; 27-30 min: 5% B.

Method B: Phenomenex Luna C18 column (250 mm×10 mm, 100 Å, AXIA packed) at a flow rate of 5 mL/min. Gradient: 0-1 min: 5% B; 1-2 min: 5-20% B; 2-25 min: 20-30% B; 25-26 min: 30-95% B; 26-28 min: 95% B; 28-29 min: 95-5% B; 29-30 min: 5% B. Analytical HPLC was carried out on a Phenomenex Luna 5 μm C18 column (150 mm×3 mm, 100 Å, AXIA packed) at a flow rate of 0.8 mL/min using either a Shimadzu HPLC-20AR equipped with a binary gradient pump, UV-vis detector, autoinjector, and Laura radiodetector or Agilent 1260 Infinity II HPLC. UV absorption was recorded at 254 nm.

Method C: Gradient: 0-2 min: 5% B; 2-14 min: 5-95% B; 14-16 min: 95% B; 16-16.5 min: 95-5% B; 16.5-20 min 5% B.

### 2. Synthetic Scheme

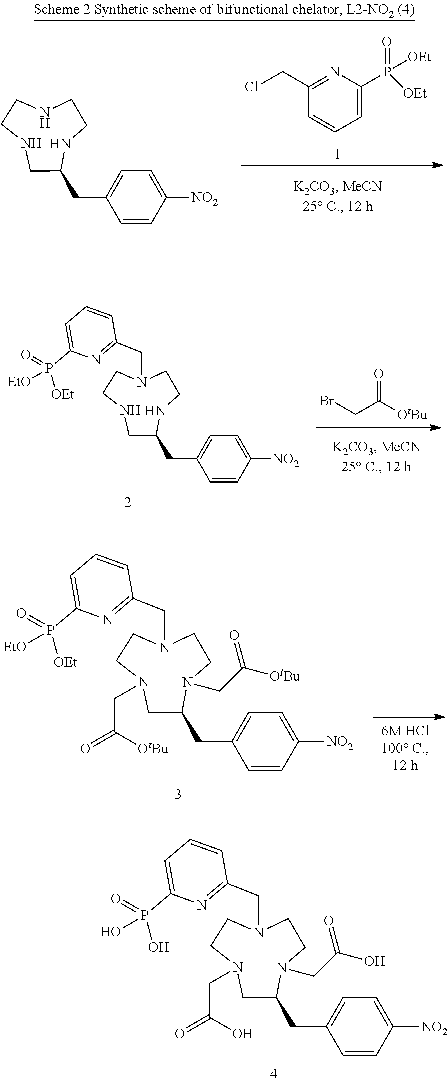 [SMILES: O=[N+]([O-])c1ccc(C[C@H]2CNCCNCCN2)cc1 ; CCOP(=O)(OCC)c1cccc(CCl)n1 ; CCOP(=O)(OCC)c1cccc(CN2CCNC[C@H](Cc3ccc([N+](=O)[O-])cc3)NCC2)n1 ; CC(C)(C)OC(=O)CBr ; CCOP(=O)(OCC)c1cccc(CN2CCN(CC(=O)OC(C)(C)C)C[C@H](Cc3ccc([N+](=O)[O-])cc3)N(CC(=O)OC(C)(C)C)CC2)n1 ; O=C(O)CN1CCN(Cc2cccc(P(=O)(O)O)n2)CCN(CC(=O)O)[C@@H](Cc2ccc([N+](=O)[O-])cc2)C1]

### 3. Synthetic Procedures

Diethyl (6-(chloromethyl)pyridine-2-yl)phosphonate (1) was synthesized in two steps according to a previously published procedure (Salaam et al. 2018).

### Diethyl (S)-(6-((X-(4-nitrobenzyl)-1,4,7-triazonan-1-yl)methyl) pyridin-2-yl)phosphonate (2a-b)

To a solution of(S)-2-(4-nitrobenzyl)-1,4,7-triazonane (30.0 mg, 0.1135 mmol) in dry acetonitrile was added K₂CO₃ (78.3 mg, 0.5675 mmol) to afford a suspension. The mixture was cooled to 0° C. in an ice-water bath and a solution of 2 (29.8 mg, 0.1135 mmol) in dry acetonitrile was added dropwise. Following stirring at room temperature for 12 hours, the reaction mixture was filtered, and the filtrate was concentrated in vacuo. This procedure afforded two of three possible structural isomers, where X=2, 3 or 5; X=5 is shown. Reverse phase preparative HPLC (Method B) enabled purification and separation of structural isomers 2a (6.9 mg, 0.0140 mmol) and 2b (7.4 mg, 0.0151 mmol) in a combined yield of 26%.

Di-tert-butyl 2,2′-(7-((6-(diethoxyphosphoryl)pyridin-2-yl)methyl)-X-(4-nitrobenzyl)-1,4,7-triazonane-1,4-diyl) (S)-diacetate (3a-b). X=2, 5, or 6. X=2 is shown.

A suspension of 2a (6.9 mg, 0.0140 mmol) or 2b (7.4 mg, 0.0151 mmol), tert-butyl bromoacetate (3.5 μL, 0.028 mmol or 4.0 μL, 0.030 mmol) and K₂CO₃ (12.7 mg, 0.092 mmol or 10.4 mg, 0.075 mmol) in dry acetonitrile was stirred at room temperature for 12 hours. Upon completion, the reaction mixture was filtered, and the filtrate was concentrated in vacuo. The crude product was purified with reverse phase preparative HPLC (Method B) to afford 3a (4.4 mg, 0.00612 mmol) in 44% yield and 3b (4.0 mg, 0.00556 mmol) in 37% yield.

(S)-2,2′-(X-(4-nitrobenzyl)-7-((6-phosphonopyridin-2-yl)methyl)-1,4,7-triazonane-1,4-diyl)diacetic acid (4a-b). X=2, 5, or 6. X=2 is shown.

Compound 3a (4.4 mg, 0.00612 mmol) and 3b (4.0 mg, 0.00556 mmol) were independently dissolved in 6 M HCl and refluxed for 14 hours. Solvent was removed under vacuum to afford 4a and 4b as off-white solids in HCl salt, respectively, and used without further purification.

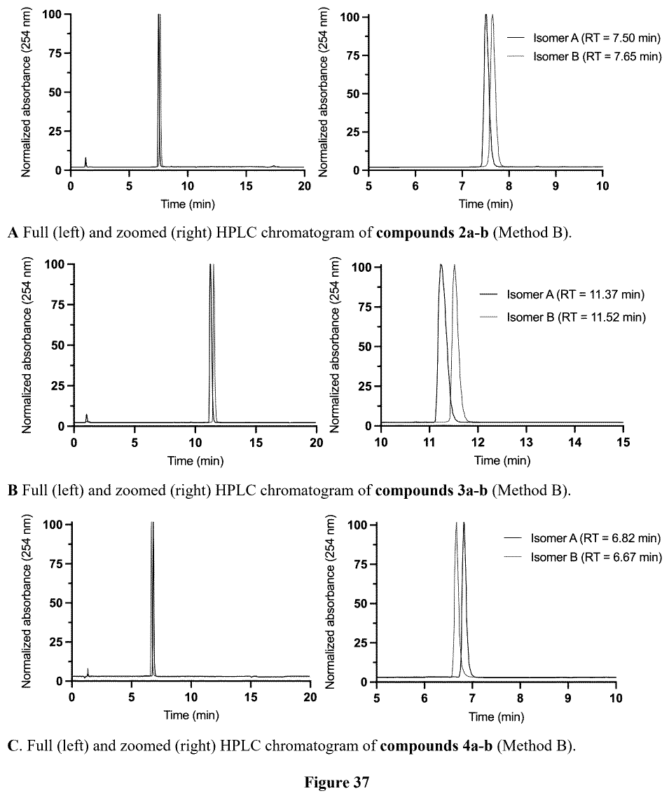

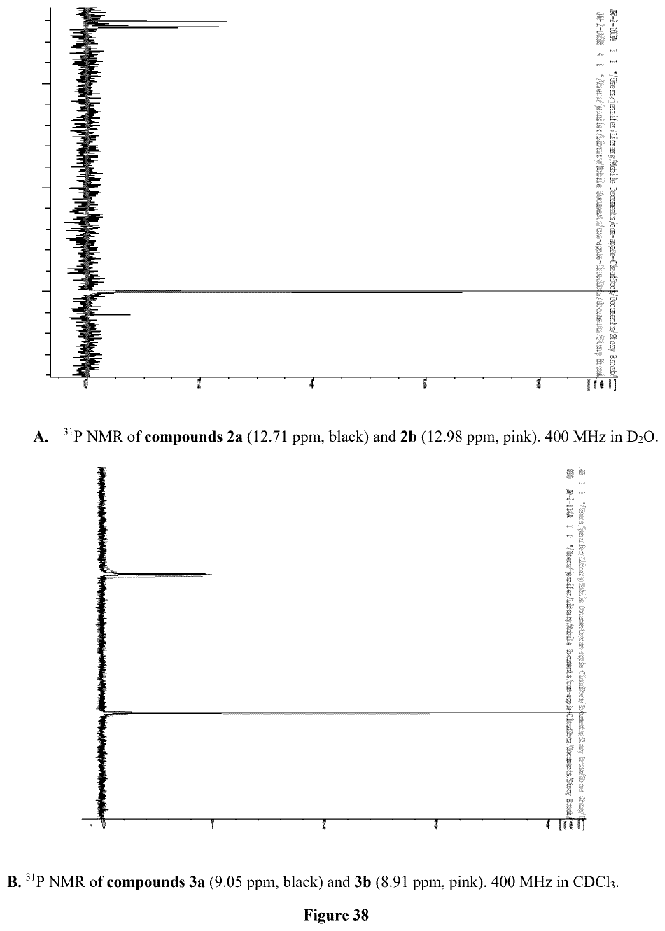

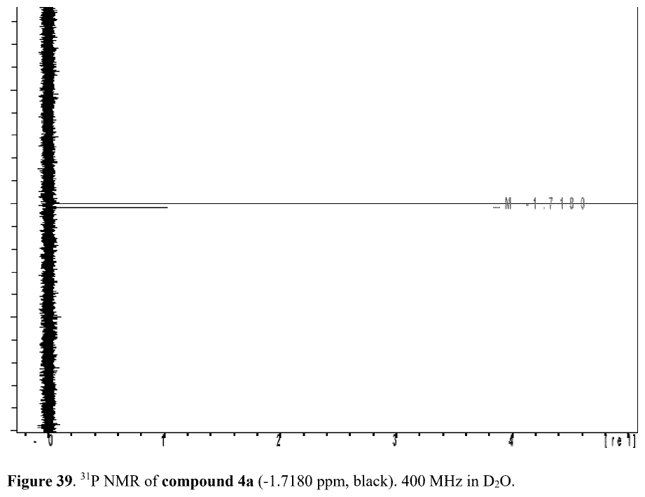

### 4. Characterization Data was Shown in FIG. 37-39

### III. Synthesis of L3, 6-((4-(carboxymethyl)-7-(phosphonomethyl)-1,4,7-triazonan-1-yl)methyl)picolinic acid

### 1. Experimental Procedures

All HPLC purification and analytical methods were conducted using a binary solvent system in which solvent A was water+0.1% TFA and solvent B was MeCN+0.1% TFA. Preparative HPLC was carried out using a Shimadzu HPLC-20AR equipped with a binary gradient pump, UV-vis detector, and manual injector. UV absorption was recorded at 254 nm.

Method A: Phenomenex Luna C18 column (250 mm×21.2 mm, 100 Å, AXIA packed) at a flow rate of 15 mL/min. Gradient: 0-1 min: 5% B; 1-14 min: 5-50% B; 14-23 min: 50-95% B; 23-26 min: 95% B; 26-27 min: 95-5% B; 27-30 min: 5% B. Analytical HPLC was carried out on a Phenomenex Luna 5 μm C18 column (150 mm×3 mm, 100 Å, AXIA packed) at a flow rate of 0.8 mL/min using either a Shimadzu HPLC-20AR equipped with a binary gradient pump, UV-vis detector, autoinjector, and Laura radiodetector or Agilent 1260 Infinity II HPLC. UV absorption was recorded at 254 nm.

Method B: Gradient: 0-2 min: 5% B; 2-14 min: 5-95% B; 14-16 min: 95% B; 16-16.5 min: 95-5% B; 16.5-20 min 5% B.

### 2. Synthetic Schemes

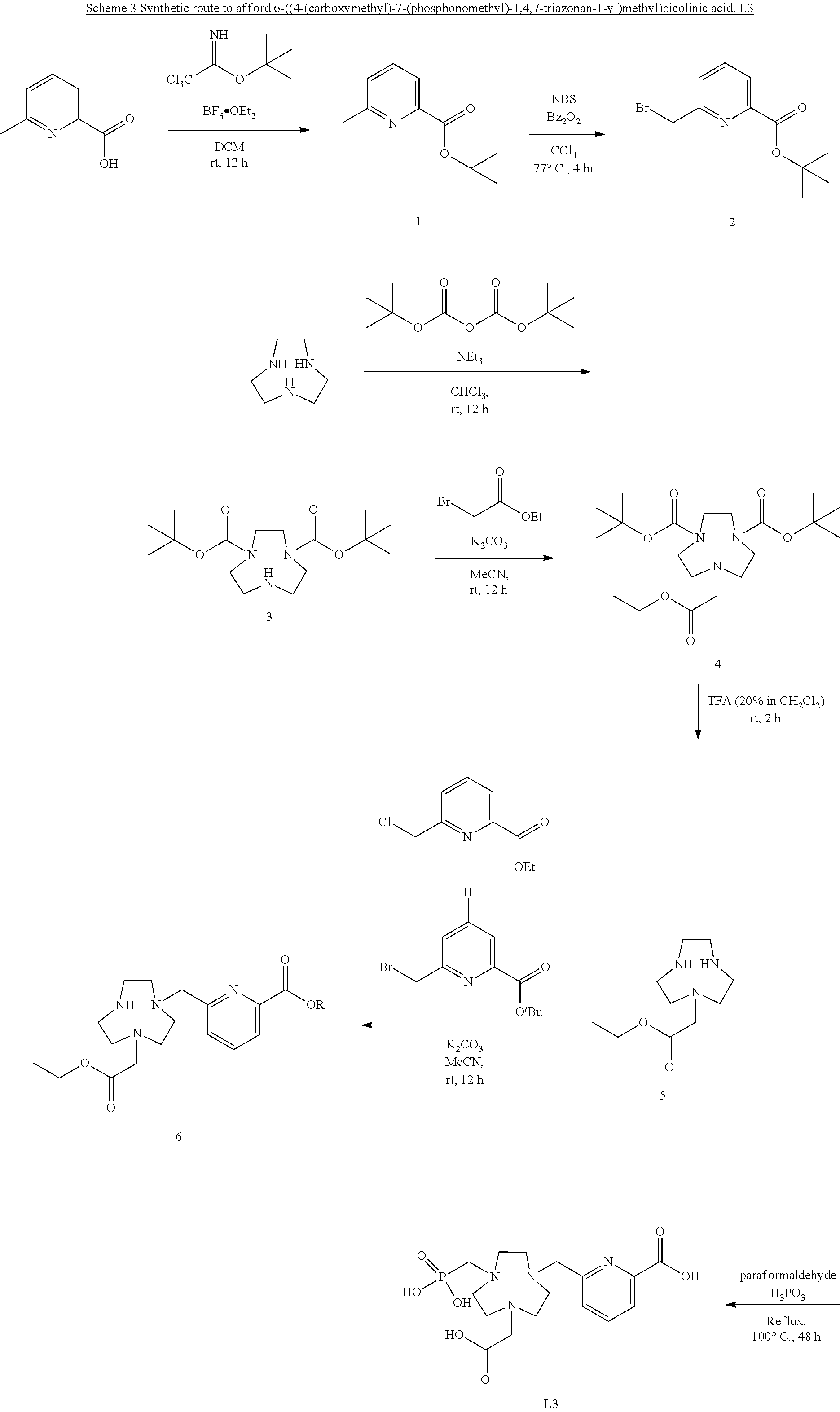

### 3. Synthetic Procedures

### tert-butyl 6-methylpicolinate (1)

6-methylpicolinic acid (298.1 mg, 2.18 mmol) was dissolved in dichloromethane (15 mL), followed by addition of tert-butyl 2,2,2-trichloroacetimidate (783 μL, 4.38 mmol) and boron trifluoride, BF₃·OEt₂ (43 μL) The reaction mixture was stirred overnight at room temperature and monitored via TLC (90:10 DCM: MeOH, UV visualized on silica plates). Upon completion, the reaction mixture was filtered, and the filtrate was concentrated under reduced pressure. The resulting solid was resuspended in hexanes, filtered and the solid was washed with hexanes (3×10 mL). The crude product was purified via column chromatography (silica solid phase, 0-5% MeOH in DCM mobile phase) to afford the product as a yellow oil in a 32% yield.

### tert-butyl 6-(bromomethyl)picolinate (2)

Compound 1 (133.7 mg, 0.69 mmol) was dissolved in 2 mL carbon tetrachloride to afford a yellow solution. N-bromosuccinimide (98.5 mg, 0.8 eq, 0.55 mmol) and benzoyl peroxide (502.76 mg, 2.08 mmol based on 75% w/w in water) were added and the reaction mixture was heated to reflux for 4 hours. The reaction was monitored via TLC (mobile phase: 9:1 hexane:ethyl acetate, UV visualized). The crude mixture was filtered, and the filtrate concentrated to dryness. Needle-like crystals were resuspended in DCM, filtered, dried, and purified via column chromatography (silica solid phase, 0-25% ethyl acetate in hexane).

### di-tert-butyl 1,4,7-triazonane-1,4-dicarboxylate (3)

1,4,7-triazacyclononane (TACN, 129.4 mg, 1.00 mmol) was dissolved in chloroform (30 mL), followed by the addition of triethyl amine (279 μL, 2.00 mmol). A solution of di-tert-butyl dicarbonate (460 μL, 2.00 mmol) in chloroform (9 mL) was added dropwise to the reaction mixture to afford a final TACN concentration of 0.02 M. The solution was stirred at room temperature for 2 hours. Upon completion, the solvent was removed under reduced pressure and the resulting residue was partitioned between 10% NaOH (10 mL) and diethyl ether (30 mL). The organic layer was then sequentially washed with 10% NaOH (4×10 mL) and water (3×10 mL). The organic layer was dried over Na₂SO₄, filtered, and concentrated to afford a transparent yellow oil in a 78% yield (Chong, et al. 2018).

### di-tert-butyl 7-(2-ethoxy-2-oxoethyl)-1,4,7-triazonane-1,4-dicarboxylate (4)

Compound 3 (135 mg, 0.41 mmol) was dissolved in dry acetonitrile, followed by addition of oven dried K₂CO₃ (185 mg, 1.34 mmol) to afford a suspension. Ethyl bromoacetate (49.6 μL, 0.45 mmol) was added, and the reaction was allowed to stir overnight at room temperature. The reaction mixture was filtered and concentrated to afford a yellow oil (crude yield=84%).

### ethyl 2-(1,4,7-triazonan-1-yl)acetate (5)

Compound 4 (143.2 mg, 0.34 mmol) was dissolved in 20% trifluoroacetic acid in dichloromethane (4 mL) and allowed to stir at room temperature for 2 hours. Following completion, the reaction mixture was concentrated under reduced pressure.

### Tert-butyl 6-((4-(2-ethoxy-2-oxoethyl)-1,4,7-triazonan-1-yl)methyl)picolinate (6)

Compound 5 (41.7 mg, 0.19 mmol) was dissolved in dry acetonitrile (5 mL), followed by addition of oven dried K₂CO₃ (130.8 mg, 0.95 mmol) to afford a suspension. The reaction mixture was cooled to 0° C. over an ice-water bath, followed by dropwise addition of compound 2 in acetonitrile (2 mL). The reaction mixture was stirred at room temperature overnight. Upon completion, the reaction was filtered, concentrated and HPLC purified (Method A). The product was afforded as red/brown oil in 45% yield following lyophilization.

### 6-((4-(carboxymethyl)-7-(phosphonomethyl)-1,4,7-triazonan-1-yl)methyl)picolinic acid (L3)

To a solution of compound 6 (26 mg, 0.064 mmol) in 6 M trace metal grade HCl (1.65 mL, 0.04 M) was added phosphorous acid (64 μL, 1.28 mmol) and the reaction mixture heated to reflux. Paraformaldehyde (8.7 mg, 0.29 mmol) was added at the reaction stirred overnight. The reaction mixture was filtered, concentrated, and HPLC purified (Method A). The desired product was obtained in a 29% yield.

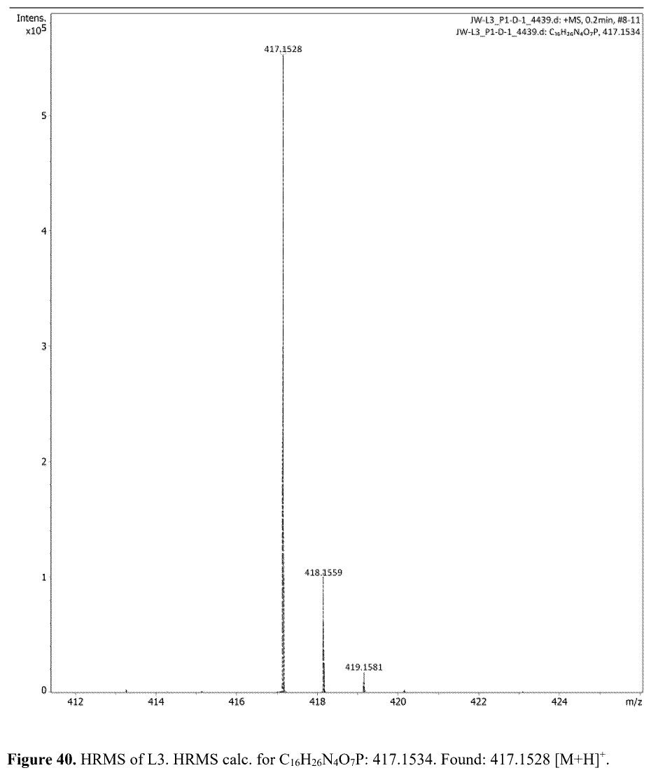

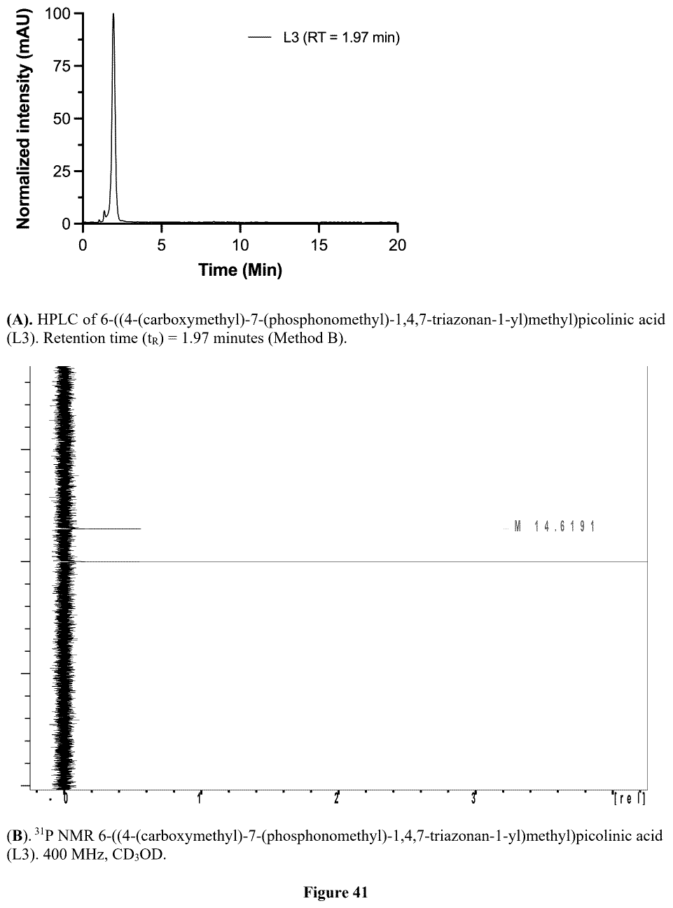

### 4. Characterization Data was Shown in FIGS. 40-41

### IV. Synthetic Scheme of Picaga-HSA

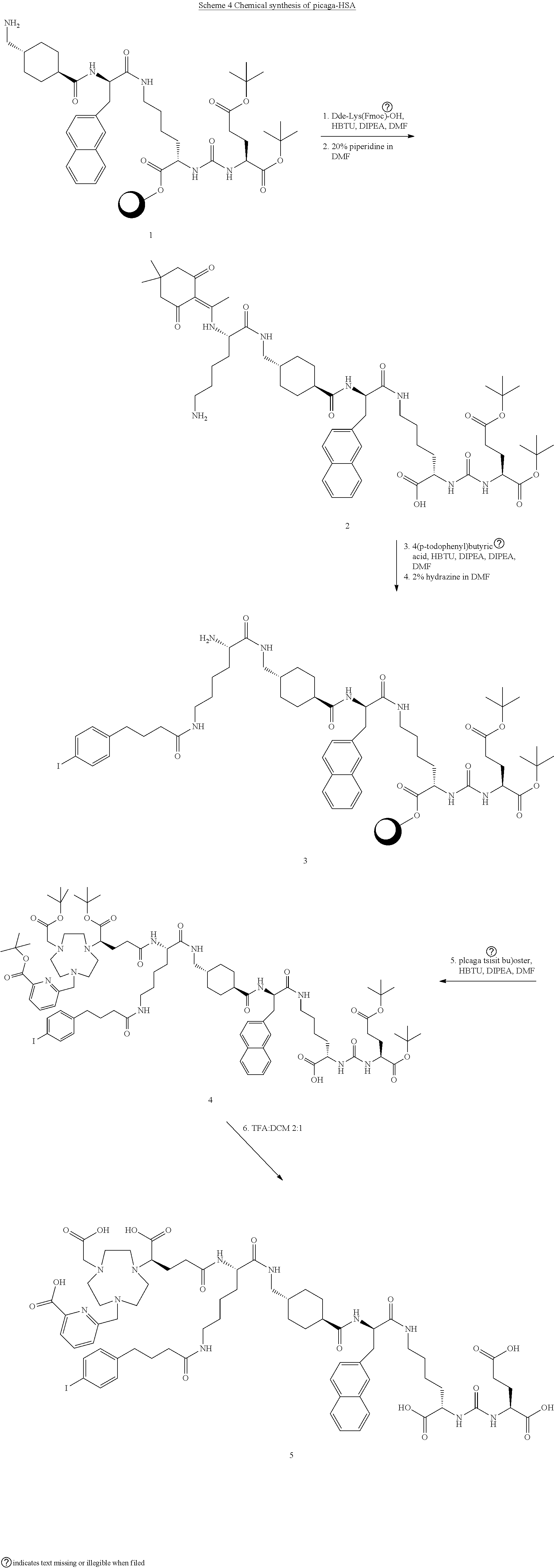

### V. Synthetic Scheme of Additional Compounds

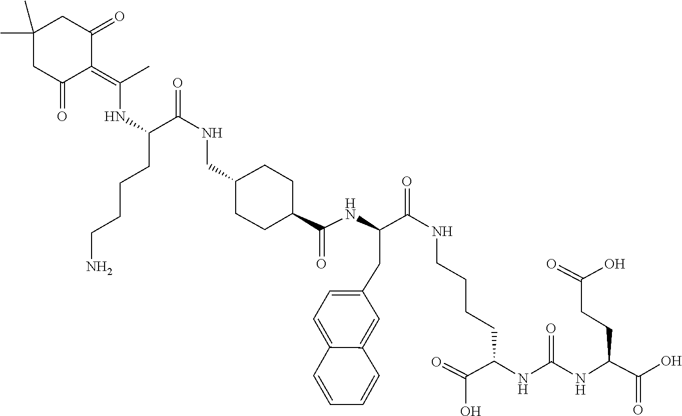 [SMILES: CC(N[C@@H](CCCCN)C(=O)NC[C@H]1CC[C@H](C(=O)N[C@H](Cc2ccc3ccccc3c2)C(=O)NCCCC[C@H](NC(=O)N[C@@H](CCC(=O)O)C(=O)O)C(=O)O)CC1)=C1C(=O)CC(C)(C)CC1=O]

(2S)-2-({[(2S)-1,5-bis(tert-butoxy)-1,5-dioxopentan-2-yl]carbamoyl}amino)-6-[(2R)-3-(naphthalen-2-yl)-2-{[(1r,4r)-4-{[(2S)-6-amino-2-{[1-(4,4-dimethyl-2,6-dioxocyclohexylidene)ethyl|amino}hexanamido]methyl}cyclohexyl]formamido}propanamido]hexan oic acid (2). Compound 1 on 2-chlorotrityl resin beads (0.212 mmol, 1.0 eq) and HBTU (0.241 g, 0.636, 3.0 eq) were dissolved in DMF (6 mL), DIPEA (0.137 g, 1.06 mmol, 5.0 eq) was added. Dde-Lys (Fmoc)-OH (0.169 g, 0.318 mmol, 1.5 eq) was added and reaction mixture was stirred overnight at room temperature. Resin beads were washed four times with dichloromethane (5.0 mL) and four times with DMF (5.0 mL). Fmoc protecting group was removed by shaking resin beads in 20% piperidine in DMF for 45 minutes (5.0 mL). Resin beads were washed four times with DMF (5.0 mL). 2 was cleaved from the resin in a 50% TFA: DCM mixture (1.0 mL). Supernatant was transferred to a vial to afford tert-butyl deprotected 2 in solution. Calculated monoisotopic mass for afford tert-butyl deprotected 2 (C₄₉H₆₉N₇O₁₂): 947.50; found m/z=948.6 [M+H]⁺.

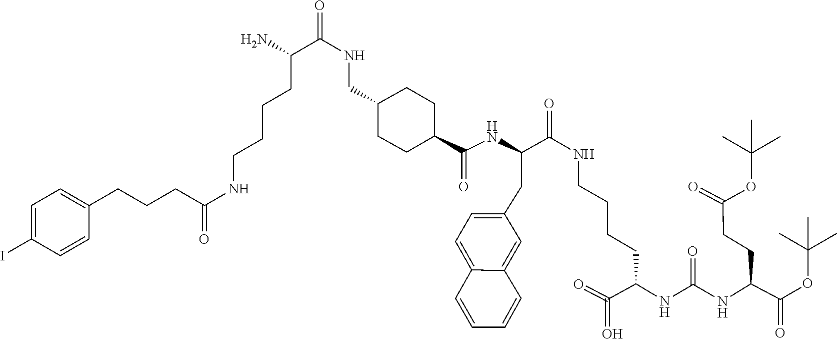

(2S)-2-({[(2S)-1,5-bis(tert-butoxy)-1,5-dioxopentan-2-yl]carbamoyl}amino)-6-[(2R)-3-(naphthalen-2-yl)-2-{[(1r,4r)-4-{[(2S)-2-amino-6-[4-(4-iodophenyl)butanamido]hexanamido]methyl}cyclohexyl]formamido}propanamido]hexanoic acid (3). Compound 2 on 2-chlorotrityl resin beads (0.212 mmol, 1.0 eq) and HBTU (0.241 g, 0.636, 3.0 eq) were dissolved in DMF (6 mL), DIPEA (0.137 g, 1.06 mmol, 5.0 eq) was added. 4-(p-Iodophenyl) butyric acid (0.0923 g, 0.318 mmol, 1.5 eq) was added and reaction mixture was stirred overnight at room temperature. Resin beads were washed four times with dichloromethane (5.0 mL) and four times with dimethylformamide (5.0 mL). Dde-protecting group was removed by shaking resin beads in 2% hydrazine in DMF for 1 hour. Resin beads were washed four times with DMF (5.0 mL). 3 was cleaved from the resin in a 1% TFA: DCM mixture (1.0 mL). Supernatant was transferred to a vial to afford 3 in solution. Calculated monoisotopic mass for 3 (C₅₇H₈₂IN₇O₁₁): 1167.51; found m/z=1168.5 [M+H]⁺.

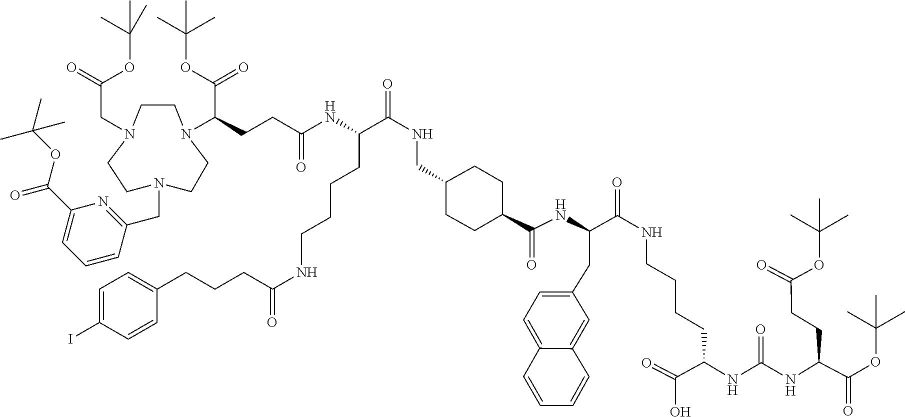 [SMILES: CC(C)(C)OC(=O)CC[C@H](NC(=O)N[C@@H](CCCCNC(=O)[C@@H](Cc1ccc2ccccc2c1)NC(=O)[C@H]1CC[C@H](CNC(=O)[C@H](CCCCNC(=O)CCCc2ccc(I)cc2)NC(=O)CC[C@H](C(=O)OC(C)(C)C)N2CCN(CC(=O)OC(C)(C)C)CCN(Cc3cccc(C(=O)OC(C)(C)C)n3)CC2)CC1)C(=O)O)C(=O)OC(C)(C)C]

(2S)-2-({[(2S)-1,5-bis(tert-butoxy)-1,5-dioxopentan-2-yl]carbamoyl}amino)-6-[(2R)-3-(naphthalen-2-yl)-2-{[(1r,4r)-4-{[(2S)-2-[(4R)-5-(tert-butoxy)-4-{4-[2-(tert-butoxy)-2-oxoethyl]-7-({6-[(tertbutoxy)carbonyl]pyridin-2-yl}methyl)-1,4,7-triazonan-1-yl}-5-oxopentanamido]-6-[4-(4-iodophenyl)butanamido]hexanamido]methyl}cyclohexyl]formamido}propanamido]hexanoic acid (4). Compound 3 2-chlorotrityl resin beads (0.086 mmol, 1.0 eq) and HBTU (0.098 g, 0.258, 3.0 eq) were dissolved in DMF (1 mL), DIPEA (0.055 g, 0.430 mmol, 5.0 eq) was added. Ditert-butyl 2-{3-[(R)-4-(5-aminopentylamino)-4-oxo-1-tert-butoxycarbonylbutyl]ureido}glutarate (0.086 g, 0.130 mmol, 1.5 eq) was added and reaction mixture was stirred overnight at room temperature. Resin beads were washed four times with dichloromethane (5.0 mL) and four times with dimethylformamide (5.0 mL). 4 was cleaved from the resin in a 1% TFA: DCM mixture (1.0 mL). Supernatant was transferred to a vial and solvent was removed in vacuo. Product was purified by semi-preparative HPLC (Method A) to afford 4 (0.0085 g, 0.005 mmol, 6%) as an off-white solid. Calculated monoisotopic mass for 4 (C₈₉H₁₃₂IN₁₁O₁₈): 1769.88; found: m/z=1770.8858 [M+H]⁺, 886.4491 [M+2H]²⁺.

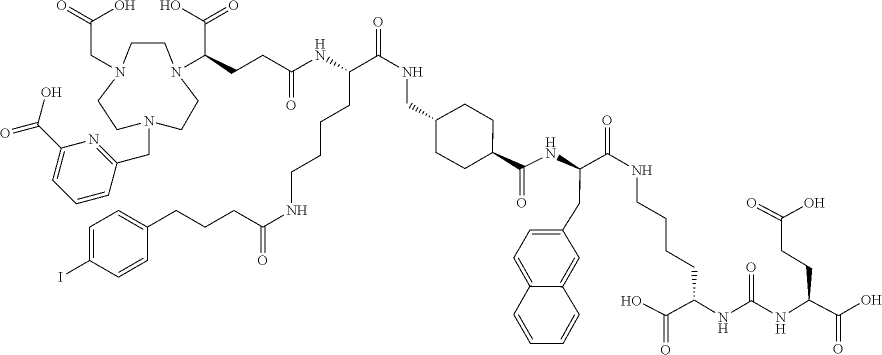 [SMILES: O=C(O)CC[C@H](NC(=O)N[C@@H](CCCCNC(=O)[C@@H](Cc1ccc2ccccc2c1)NC(=O)[C@H]1CC[C@H](CNC(=O)[C@H](CCCCNC(=O)CCCc2ccc(I)cc2)NC(=O)CC[C@H](C(=O)O)N2CCN(CC(=O)O)CCN(Cc3cccc(C(=O)O)n3)CC2)CC1)C(=O)O)C(=O)O]

(2S)-2-({[(1S)-1-carboxy-5-[(2R)-3-(naphthalen-2-yl)-2-{[(1r,4r)-4-{[(2S)-2-[(4R)-4-carboxy-4-[4-(carboxymethyl)-7-[(6-carboxypyridin-2-yl)methyl]-1,4,7-triazonan-1-yl]butanamido]-6-[4-(4-iodophenyl)butanamido]hexanamido]methyl}cyclohexyl]formamido}propanamido]pentyl]carbamo yl}amino) pentanedioic acid, picaga-HSA (5). Compound 4 (0.007 g, 0.004 mmol, 1 eq) was dissolved into a solution of 2:1 TFA and DCM (0.3 mL). The reaction mixture was stirred overnight at room temperature. Solvent was removed in vacuo and 5 isolated as an off-white solid (0.0064 g, 0.004 mmol, 86%). Calculated monoisotopic mass for 5 (C₆₉H₉₂IN₁₁O₁₈): 1489.57; found: m/z=1490.6 [M+H]⁺, 745.9 [M+2H]²⁺.

### Example 7

### A. Ligand Concentration Determination (Titration)

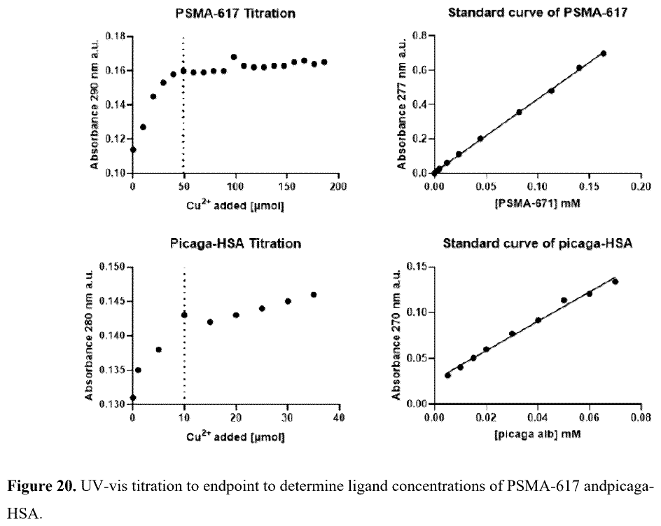

To determine the concentration and molar absorptivity of picaga-HSA and PSMA-617, a spectrophotometric titration was carried out with Cu²⁺. The formation of [Cu(picaga-HSA)]⁻ or [Cu(PSMA-617)]⁻ was monitored at 280 nm or 290 nm using a 1 cm path length cuvette and a NanoDrop spectrophotometer. The pH was adjusted to 5.5 using 0.25 M ammonium acetate buffer. 100 μM ligand stock solutions were titrated with addition of 98 μM Cu²⁺ aliquots (as determined by ICP-OES) to determine the concentration of ligand by equivalents of Cu²⁺. The titration endpoint was determined by the inflection point of the change to the absorbance intensity at 280 nm or 290 nm, diagnostic of complex formation, was detected. A standard curve from A standard curve from 0.005 to 0.07 mM picaga-HSA or 0.00049 to 0.16 mM PSMA-617 was measured at 277 nm and the slope was determined using simple linear regression in Graph Pad Prism. The molar absorptivity (ε) was calculated from the slope of the standard curve as ε=1603 M⁻¹ cm⁻¹ for picaga-HSA. The molar absorptivity (ε) was calculated from the slope of the standard curve as ε=4268 M⁻¹ cm⁻¹ for PSMA-617. (See FIG. 20)

### B. Preparation of Non-Radioactive Lutetium Complexes

^{nat}Lu-complexes were formed in a 0.40 M solution of ammonium acetate at pH 5.5 at 80° C. for 30 minutes. Complex formation was monitored and characterized by HPLC-MS as described below.

Lu-PSMA-617. H₃PSMA-617 in deionized water (70 μL) and LuCl₃·6H₂O in deionized water (5 μL) was added to a 0.40 M solution of ammonium acetate at pH 5.5 (500 μL). The resulting solution was heated at 80° C. for 30 minutes. Complex formation was confirmed by mass spectrometry. Calculated monoisotopic mass for (C₄₉H₆₈LuN₉O₁₆): 1213.42; found: m/z=1214.4 [M+H], m/z=607.9 [M+2H]. Rₜ of ¹⁷⁷Lu radiolabeled product on HPLC (Method B: gradient: A: H₂O, 0.1% TFA, B: CH₃CN. 5-100% B gradient 20 min): 7.15±0.2 minutes (n≥9).

Lu-(picaga)-HSA. H₃(picaga)-DUPA in dimethylsulfoxide (100 μL) and LuCl₃·6H₂O in deionized water (5 μL) in was added to a 0.40 M solution of ammonium acetate at pH 5.5 (500 μL). The resulting solution was heated at 80° C. for 30 minutes. Complex formation was confirmed by mass spectrometry. Calculated monoisotopic mass for (C₆₉H₈₉ILuN₁₁O₁₈): 1661.48; found: m/z=1662.6 [M+H], m/z=831.8 [M+2H]. Rₜ of ¹⁷⁷Lu radiolabeled product on HPLC (Method B: gradient: A: H₂O, 0.1% TFA, B: CH₃CN. 5-100% B gradient 20 min): 9.01±0.2 minutes (n≥9).

### Example 8. Determination of Ligand Concentration

To determine the concentration and molar absorptivity of picaga-HSA and PSMA-617, a spectrophotometric titration was carried out with Cu²⁺. The formation of [Cu(picaga-HSA)]⁻ or [Cu(PSMA-617)] was monitored at 280 nm or 290 nm using a 1 cm path length cuvette and a NanoDrop spectrophotometer. The pH was adjusted to 5.5 using 0.25 M ammonium acetate buffer. 100 μM ligand stock solutions were titrated with addition of 98 μM Cu²⁺ aliquots (as determined by ICP-OES) to determine the concentration of ligand by equivalents of Cu²⁺. The titration endpoint was determined by the inflection point of the change to the absorbance intensity at 280 nm or 290 nm, diagnostic of complex formation, was detected. A standard curve from 0.005 to 0.07 mM picaga-HSA or 0.00049 to 0.16 mM PSMA-617 was measured at 277 nm and the slope was determined using simple linear regression in Graph Pad Prism. The molar absorptivity (ε) was calculated from the slope of the standard curve as ε=1603 M⁻¹ cm⁻¹ for picaga-HSA. The molar absorptivity (ε) was calculated from the slope of the standard curve as ε=4268 M⁻¹ cm⁻¹ for PSMA-617.

### Example 9. Binding Affinity to PSMA

Stock solutions of Lu-(picaga)-HSA in (DMSO: H₂O, 1:5) were prepared and concentrations were determined by ICP-OES in accordance with previously published method. In brief, 12 concentrations ranging 1 mM-10 pM Lu (picaga)-HSA, and 1 mM-10 pM (DCFPyL) N-{[(1S)-1-carboxy-5-{[(6-fluoro-3-pyridinyl) carbonyl]amino}-pentyl]carbamoyl}-L-glutamic acid were used as cold displacers to ^{99m}Tc(CO)₃-MIP-1427, which was synthesized according to literature procedure (Hillier, S. M. et al. 2013).

The Kᵢ values were calculated using the equation below (Szabo, Z. et al. 2015), where the Kᵢ value of DCFPyL was given as 1.1 nM. The Kᵢ was determined by nonlinear regression analysis using GraphPad Prism software.

$${K}_{i} = \frac{{IC}_{50}}{1 + \frac{[ S ]}{{K}_{M}}}$$

### Example 10. Binding to Human Serum Albumin

To measure HSA binding of the complexes, a 0.1 mM solution (determined by ICP-OES) of the corresponding ^{nat}Lu complex in 4.5% w/v HSA was prepared and pipetted into an Amicon Ultra-0.5 Centrifugal Filter Unit (50 KDa cutoff, Millipore, UFC500396). The mixture was incubated at 37° C. for 15 min and subsequently centrifuged at 12000 rpm for 10 min. Binding is determined by measurement of Lu content in the filtrate by ICP-OES and compared to non-specific binding to the filter in absence of HSA.

### Example 11. Preparation of Radioactive Lutetium Complexes

### ¹⁷⁷Lu Radiolabeling Protocol for Picaga-HSA and PSMA-617.

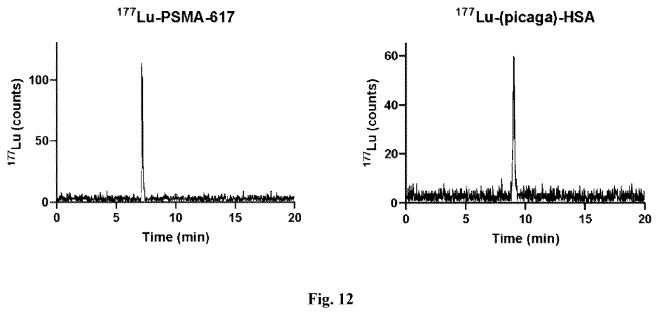

The general radiolabeling protocol was used to radiolabel H₃(picaga)-HSA and H₃PSMA-617. Ligand was dissolved in dimethylsulfoxide to produce a stock solution. 10 nmol (PSMA-617) or 20 nmol (picaga-HSA) in 0.03 mL of the stock solution was added to an Eppendorf tube and 0.07 mL of 0.4 M ammonium acetate pH 5.5 was added to the solution. ¹⁷⁷Lu was received in 0.05 M HCl (63 MBq) Activity was added to ligand solution and tube was placed in a heating block at 80° C. Tubes were intermittently vortexed and 5 μL was removed after 30 minutes to check complexation by radio-HPLC. The radiolabeled compounds were diluted in sterile PBS pH 7.4 at an activity concentration of 37 MBq/mL ready for injection. Radio-HPLC traces are shown in FIG. 12. Rt (method B): ¹⁷⁷Lu-PSMA-617 Rt(min)=7.15±0.02 (n=9). ¹⁷⁷Lu-picaga-HSA Rt(min)=9.01±0.02 (n=9).

### Example 12. Dose Formulation Stability of Radio-Labeled Compound

The stability for ¹⁷⁷Lu-radiolabeled ligands was evaluated by radio-HPLC for radiolytic degradation and decomplexation at relevant time points concentrations for dose preparation, storage, and administration. (picaga)-HSA was radiolabeled as previously described at a specific activity of 0.08 mCi/nmol (3.0 MBq/nmol) and diluted in PBS. A 0.1 mL aliquot was removed, stored at room temperature, and tested for stability by radio-HPLC. The activity concentration at t=0 was 0.12 mCi in 0.1 mL.

TABLE 1
Time-dependent complex stability in dose formulation
(DMSO:NH₄OAc:phosphate buffered saline).

| Ligand | 4 h | 24 h | 48 h | 14 d |
|---|---|---|---|---|
| ¹⁷⁷Lu-(picaga)-HSA | >99% | >99% | >99% | 74% |
| ¹⁷⁷Lu-PSMA-617 | >99% | >99% | >99% | 80% |

### Example 13. Determination of 1-Octanol/PBS pH 7.4 Distribution Coefficients (Log D_{7.4})

The distribution coefficient was determined by liquid-liquid extraction and phase separation by centrifugation in 1-octanol and phosphate-buffered saline at pH 7.4 (PBS). 10 μL of either ¹⁷⁷Lu-PSMA-617 or ¹⁷⁷Lu-(picaga)-HSA in 0.4 M NH₄OAc buffer was added to 490 μL PBS pH 7.4 (5 μCi, 0.3 nmol) in polypropylene tubes. 500 μL of 1-octanol was added and each tube was vortexed for two minutes. Tubes were centrifuged at 1600 ref for 3 minutes to accelerate phase separation. 50 μL was removed from each layer and radioactivity was measured on a gamma counter (Perkin Elmer Wallac Wizard 1470). The distribution coefficients were calculated as the logarithm of the ratio of the counts per minute (cpm) measured in the organic phase (1-octanol) over the cpm measured in the aqueous phase (PBS pH 7.4).

TABLE 2
LogD_{7.4} values of ¹⁷⁷Lu-PSMA-617
and ¹⁷⁷Lu-(picaga)-HSA (n = 4).

| Compound | LogD_{7.4} |
|---|---|
| ¹⁷⁷Lu-PSMA-617 | −2.71 ± 0.08 |
| ¹⁷⁷Lu-(picaga)-HSA | −2.11 ± 0.03 |

### Example 14. Cell Uptake and Internalization

The binding and internalization of ¹⁷⁷Lu-(picaga)-HSA and ¹⁷⁷Lu-PSMA-617 was evaluated in PSMA+PC3 PIP cells. In brief, 5×10⁵ PC3 PiP cells are suspended, washed and aliquoted. The internalized fraction is determined using ¹⁷⁷Lu-ligand in PC3 PiP cells, with ligands radiolabeled at a specific activity of 0.08 mCi/nmol (3.0 MBq/nmol) and diluted in PBS. Cells are incubated at 37° C. for 90 minutes with a 5 μCi aliquot of the ¹⁷⁷Lu-ligand complex, followed by incubation with acidic stripping buffer (0.05 M glycine stripping buffer in 100 mM NaCl, pH 2.8) to remove surface-bound ¹⁷⁷Lu-ligand. Subsequently, cell samples were lysed by addition of NaOH (1 M, 1 mL) to release cellular contents and internalized ¹⁷⁷Lu-(picaga)-HSA. Radioactive counts are quantitated separately using a gamma well counter, with each incubation conducted in triplicate.

TABLE 3
Time dependent cell-binding and internalization.

| | % Uptake | | | | % Internalization | | |
|---|---|---|---|---|---|---|---|
| Ligand | 2 h | 4 h | 24 h | Blocking | 2 h | 4 h | 24 h |
| ¹⁷⁷Lu-(picaga)- | 12.96 ± | 10.22 ± | 21.16 ± | 8.80 ± | 44.50 ± | 64.54 ± | 73.26 ± |
| HSA | 0.26 | 1.18 | 1.41 | 4.78 | 2.00 | 7.48 | 5.72 |
| ¹⁷⁷Lu-PSMA-617 | 6.36 ± | 6.15 ± | 9.60 ± | 0.45 ± | 33.53 ± | 36.11 ± | 50.93 ± |
| | 0.43 | 0.21 | 0.11 | 0.03 | 1.40 | 2.93 | 2.14 |

### Example 15. Biodistribution

All animal experiments were conducted according to the guidelines of the Institutional Animal Care and Use Committee (IACUC) at Stony Brook Medicine. Male NCr nude mice (6 weeks, Taconic Biosciences, Rensselaer, NY) were implanted subcutaneously on the right shoulder with 0.7-0.9×10⁶ PC-3 PiP cells and on the left shoulder with 0.7-0.9×10⁶ PC-3 flu cells suspended in Matrigel (1:2). When the tumors reached 50-100 mm³, the mice were anesthetized with isoflurane, and 1.7-2.2 MBq (45-60 μCi) of the tracer (0.4-0.9 nmol) was intravenously injected via tail vein catheter. At 4, 24 and 72 h, mice were sacrificed, and select organs were harvested. Radioactivity was counted by using a gamma counter, and the radioactivity associated with each organ was expressed as % ID/g. Biodistribution data were assessed by unpaired t-tests using GraphPad Prism to determine if differences between groups were statistically significant (p<0.05).

TABLE 4
Tabulated biodistribution data.

| | | | |
|---|---|---|---|
| Organ | 2 h p.i. (% ID/g) | 24 h p.i. (% ID/g) | 72 h p.i. (% ID/g) |

¹⁷⁷Lu-(picaga)-HSA (n = 4)

| | | | |
|---|---|---|---|
| Blood | 22.10 ± 1.97 | 12.45 ± 1.43 | 6.07 ± 0.78 |
| Heart | 6.98 ± 0.51 | 4.24 ± 0.21 | 2.19 ± 0.42 |
| Liver | 4.03 ± 0.24 | 2.56 ± 0.29 | 1.58 ± 0.23 |
| Spleen | 4.83 ± 0.79 | 3.39 ± 0.32 | 2.46 ± 0.35 |
| Kidney | 14.93 ± 2.50 | 19.83 ± 1.45 | 15.66 ± 2.68 |
| Sm Int | 3.30 ± 0.51 | 2.07 ± 0.32 | 1.48 ± 0.28 |
| Muscle | 1.79 ± 0.22 | 1.05 ± 0.27 | 0.82 ± 0.08 |
| Bone | 2.65 ± 0.10 | 1.39 ± 0.43 | 1.35 ± 0.27 |
| Tumor+ | 10.18 ± 1.47 | 32.35 ± 9.15 | 41.15 ± 7.57 |
| Tumor− | 6.48 ± 1.01 | 9.24 ± 0.26 | 4.28 ± 0.73 |

¹⁷⁷Lu-PSMA-617 (n = 4)

| | | | |
|---|---|---|---|
| Blood | 0.95 ± 0.46 | 0.21 ± 0.12 | 0.03 ± 0.01 |
| Heart | 0.27 ± 0.11 | 0.09 ± 0.04 | 0.03 ± 0.01 |
| Liver | 0.29 ± 0.12 | 0.08 ± 0.04 | 0.03 ± 0.00 |
| Spleen | 0.27 ± 0.12 | 0.08 ± 0.03 | 0.04 ± 0.01 |
| Kidney | 5.12 ± 1.69 | 0.57 ± 0.25 | 0.08 ± 0.04 |
| Sm Int | 0.28 ± 0.13 | 0.06 ± 0.02 | 0.03 ± 0.01 |
| Muscle | 0.14 ± 0.05 | 0.04 ± 0.01 | 0.02 ± 0.01 |
| Bone | 0.21 ± 0.09 | 0.07 ± 0.01 | 0.05 ± 0.01 |
| Tumor+ | 11.78 ± 1.27 | 8.24 ± 2.79 | 3.65 ± 0.51 |
| Tumor− | 0.48 ± 0.18 | 0.18 ± 0.11 | 0.02 ± 0.01 |

### Example 16. Whole Body Activity Clearance

Whole body activity was measured by placing each mouse into a dose-calibrator. % Remaining activity was given by =[Aₜ/Aₜ₌₀] where Aₜ is the decay-corrected whole body activity at time t and Aₜ₋₀ is the total activity measured by dose calibrator p.i. The data were fit to a one-phase decay curve in GraphPad Prism and the biological decay constant K was calculated to be K=0.005638 h⁻¹ for ¹⁷⁷Lu-(picaga)-HSA and K=0.1415 h⁻¹ for ¹⁷⁷Lu-PSMA-617. The biological half-life is t_{1/2}=122.9 h for ¹⁷⁷Lu-(picaga)-HSA and 4.9 h for ¹⁷⁷Lu-PSMA-617.

TABLE 5
Percent activity remaining in whole body p.i.

| | ¹⁷⁷Lu-(picaga)-HSA (% | ¹⁷⁷Lu-PSMA-617 (% |
|---|---|---|
| Time (h) | activity remaining) (n = 6) | activity remaining) (n = 6) |
| 0 | 100.00 ± 0.00 | 100.00 ± 0.00 |
| 24 | 90.33 ± 7.21 | 3.35 ± 0.16 |
| 48 | 80.75 ± 6.81 | ND |
| 72 | 69.42 ± 9.59 | ND |
| 96 | 48.66 ± 9.26 | ND |
| 120 | 37.70 ± 7.58 | ND |
| 144 | 30.59 ± 4.06 | ND |
| 216 | 21.05 ± 2.47 | ND |

### Example 17. Therapy Studies with ¹⁷⁷Lu-(Picaga)-HSA and ¹⁷⁷Lu-PSMA-617

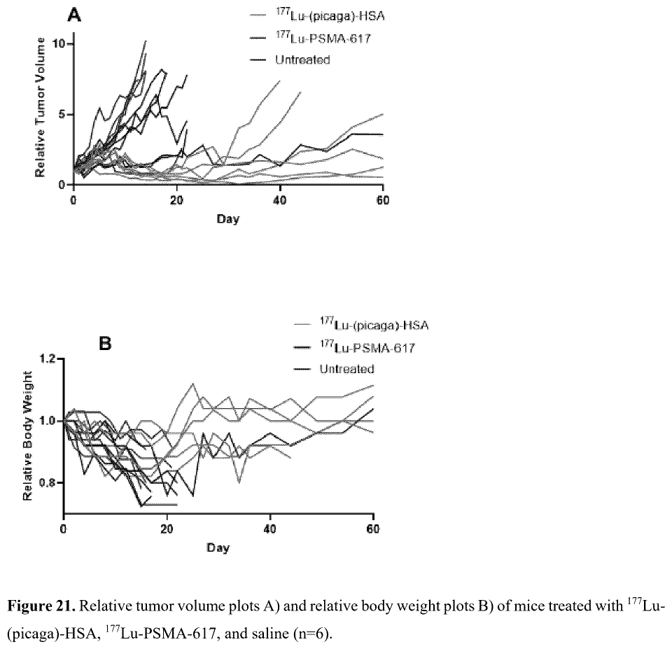

Twelve-week-old male mice were inoculated subcutaneously on the right shoulder with PSMA+PC-3 PIP cells (0.7×10⁶/mouse in 1:2 DPBS pH 7.4: Matrigel). Tumors grew nine days before treatment with an approximate volume of 100 mm³ at day 0. Three groups of mice (n=6) (cohort A, B, and C) with statistically similar body weights and tumor volumes were injected at day 0 of the therapy study. Group A received saline only. Group B and C received 3.7 MBq of the radioligand via tail vein injection (¹⁷⁷Lu-PSMA-617 or ¹⁷⁷Lu-(picaga)-HSA respectively). Following administration of radioligand or vehicle, the mice were monitored by measuring the tumor size and body weight over 60 days. Mice were euthanized when the predefined end point criteria were reached, or when the study was terminated at day 60. The relative body weight (RBW) was defined as [BWx/BW₀], where BWx is the body weight in grams at a given day x and BW₀ is the body weight in grams on day 0. The tumor dimension was determined by measuring the longest tumor axis (L) and its perpendicular axis (W) with a digital caliper. The tumor volume (V) was calculated according to the equation [V=0.5×(LW²)]. The relative tumor volume (RTV) was defined as [TVx/TV₀], where TVx is the tumor volume in mm³ at a given day x, and TV₀ is the tumor volume in mm³ at day 0 (See FIG. 21).

### Example 18. Pilot Therapy Study with ¹⁷⁷Lu-Picaga-DUPA

Twelve-week-old male mice were inoculated subcutaneously on the right shoulder with PSMA+PC-3 PIP cells (0.7×10⁵/mouse in 1:1 DPBS pH 7.4: Matrigel). Tumors grew nine days before treatment with an approximate volume of 100 mm³ at day 0. One group of mice (n=4) (cohort D) with statistically similar body weights and tumor volumes were injected at day 0 of the therapy study. Group D received 3.3 MBq of the radioligand via tail vein injection (¹⁷⁷Lu-picaga-DUPA). Following administration of radioligand, the mice were monitored by measuring the tumor size and body weight over 14 days. Mice were euthanized when the predefined end point criteria were reached, or when the study was terminated at day 14. The relative body weight (RBW) was defined as [BWₓ/BW₀], where BWx is the body weight in grams at a given day x and BW₀ is the body weight in grams on day 0. The tumor dimension was determined by measuring the longest tumor axis (L) and its perpendicular axis (W) with a digital caliper. The tumor volume (V) was calculated according to the equation [V=0.5×(LW²)]. The relative tumor volume (RTV) was defined as [TVₓ/TV₀], where TV_{z} is the tumor volume in mm³ at a given day x, and TV₀ is the tumor volume in mm³ at day 0.

### Example 19. SPECT Imaging

SPECT experiments were performed on select mice in the therapy cohorts. Scans were acquired at 4, 24, and 72 h post injection (p.i.) using a γ-Eye benchtop imaging system (BIOEMTECH, Athens, Greece). The reconstruction of SPECT data was performed using Visual-Eyes software (BIOEMTECH, Athens, Greece). Region of interest (ROI) analyses and post-processing on all images were performed using AMIDE.

### Example 20. Histopathology

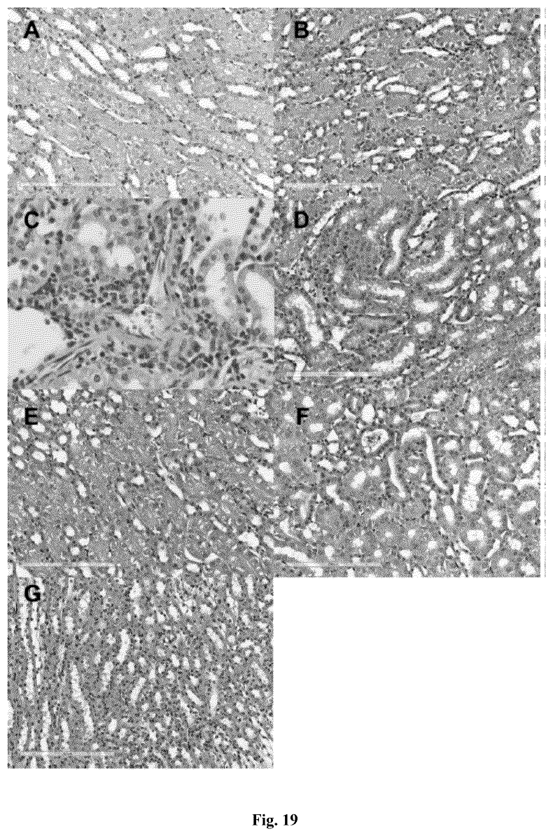

Upon the conclusion of the therapy study all surviving mice and two naive mice were sacrificed and a kidney was harvested for histopathology. H&E stain was performed, and slides were evaluated by a certified pathologist blinded to the study (FIG. 19A-G). The glomeruli were unremarkable in all cases. The tufts showed no proliferative changes, glomerulosclerosis, necrosis, or thrombosis. The tubules were also unremarkable with no signs of significant injury (tubular dilatation or epithelial cell swelling/attenuation/sloughing/mitoses), acute tubular necrosis, tubulointerstitial nephritis, infarction, or RBC/WBC/hyaline casts. No interstitial fibrosis, inflammation or edema was seen. The blood vessels were unremarkable with no indication of transmural or leukocytoclastic vasculitis, arteriosclerosis, arteriolar hyalinosis, thrombosis or thrombotic microangiopathy. The exception was one mouse treated with ¹⁷⁷Lu-(picaga)-HSA which showed mild perivascular inflammation along some blood vessels (FIG. 19C).

### Example 21. Facile Formation and In Vivo Validation of Robust Sc-¹⁸F Ternary Complexes for

### Molecular Imaging

### Experimental Procedures 1.1 Materials

All starting materials were purchased from commercial sources and used without further purification. All water used throughout radiochemistry and cold experiments was LCMS (trace metal) grade, including in the preparation of acids, bases, buffers, and stock solutions (1M HCl, 1M NaOH, 0.4M KHCO₃, 0.25 M ammonium acetate (pH 4.1) buffer, ligand, and metal stock solutions).

### 1.2 General Methods

NMR spectra (¹H, ¹⁹F, ⁴⁵Sc) were collected on a 400 MHz III Bruker instrument at 25° C. and processed using TopSpin 4.0.9. Chemical shifts are reported as parts per million (ppm). Liquid chromatography-mass spectrometry (LC-MS) was carried out on a Phenomenex Luna 5 μm C18 column (150 mm×3 mm, 100 Å, AXIA packed) at a flow rate of 0.8 mL/min using a single quadrupole Agilent 1200 Infinity II LC/MSD system equipped with a binary gradient pump, UV-vis detector, automatic injector, and an atmospheric pressure electrospray ionization (API-ES) source. UV absorption was recorded at 254 nm, and positive and negative mass spectra were collected from m/z 100-800. Gradient: 0-3 min: 5% B; 3-10 min: 5-95% B; 10-13 min: 95% B; 13-13.5 min: 95-5% B; 13.5-16 min: 5% B.

Method A: Binary solvent system (A: water+0.1% TFA; B: MeCN+0.1% TFA).

Method B: Binary solvent system (A: 10 mM ammonium acetate buffer, pH 5.6; B: MeCN). High resolution ESI mass spectrometry was carried out at the Stony Brook University Center for Advanced

Study of Drug Action (CASDA) with a Bruker Impact II UHR QTOF MS system. UV-VIS spectra were collected with the NanoDrop 1C instrument (AZY1706045). Spectra were recorded from 190 to 850 nm in a quartz cuvette with 1 cm path length. ICP-OES was carried out using an Agilent 5110 inductively coupled plasma optical emission spectrometer. A 6-point standard curve with respect to scandium or copper was used and fits were found to be at least R² of 0.999. Concentrations were back calculated to determine the stock solution concentration.

All analytical HPLC methods were carried out using a Shimadzu HPLC-20AR equipped with a binary gradient pump, UV-vis detector, autoinjector, and Laura radiodetector. UV absorption was recorded at 254 nm. Gradient: 0-2 min: 5% B; 2-14 min: 5-95% B; 14-16 min: 95% B; 16-16.5 min: 95-5% B; 16.5-20 min 5% B.

Method C: Binary solvent system (A: water+0.1% TFA; B: MeCN+0.1% TFA). Phenomenex Luna 5 μm C18 column (150 mm×3 mm, 100 Å, AXIA packed) at a flow rate of 0.8 mL/min.

Method D: Binary solvent system (A: 10 mM sodium acetate buffer, pH 5.5; B: MeCN). Phenomenex Luna 10 μm C18 column (250 mm×4 mm, 100 Å, AXIA packed) at a flow rate of 0.5 mL/min.

### 1.3 Synthesis of Ligands

The ligand 6-{[4,7-Bis(carboxymethyl)-1,4,7-triazonan-1-yl]methyl}-2-pyridinecarboxylic acid (4), H₃mpaten, and the functionalized derivative 2-{3-[(R)-1-Carboxy-4-[5-(4-carboxy-4-{7-(carboxymethyl)-4-[(6-carboxy-2-pyridyl)methyl]-1,4,7-triazonan-1-yl}butyrylamino)pentyl amino]-4-oxobutyl]ureido}glutaric acid, picaga-DUPA, were synthesized according to previously reported procedures (Vaughn et al. 2020).

### 2. Synthesis and Characterization of Isotopically Stable Complexes

### 2.1 Complexation Protocol

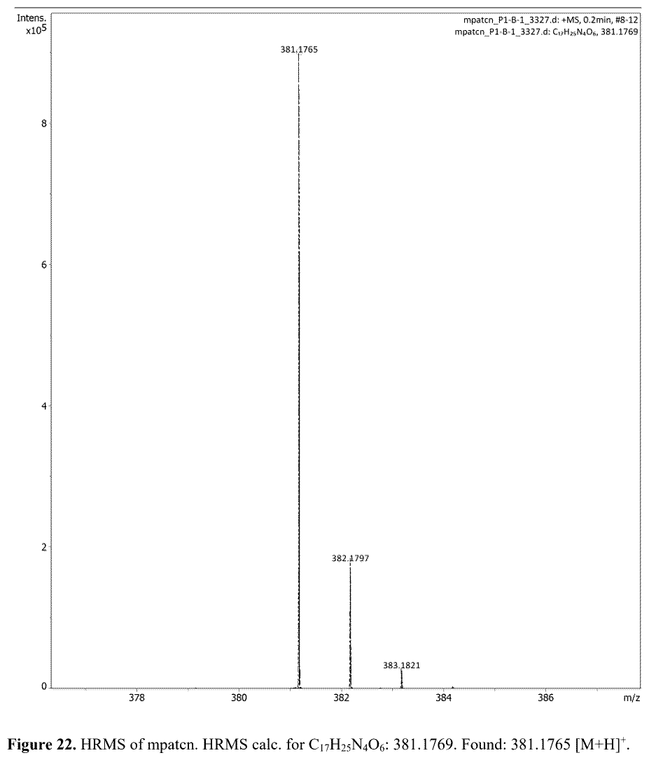

To a solution of H₃mpaten (4.2 μmol) dissolved in water, 1 equivalent of ScCl₃ salt was added. The pH was adjusted to 4.1 with 0.25 M ammonium acetate buffer. The reaction mixture was heated to 80° C. for 30 minutes, then checked via LC-MS to ensure complete complexation. Following addition of 5 equivalents NH₄F, the reaction mixture was heated to 100° C. for 30 minutes. Fluorination of Sc(mpatcn) was monitored via LC-MS (Method A), and the reaction mixture was purified via analytical HPLC (Method D). The purified complex was concentrated under reduced pressure and dissolved in 450 μL 0.25 M ammonium acetate (pH 4.1) spiked with 50 μL D₂O for ¹H, ¹⁹F and ⁴⁵Sc NMR studies. (See FIG. 22).

TABLE 6
¹H chemical shift analysis of H₃mpatcn (pH 4.5), Sc(mpatcn) (pH 4-6)
and [ScF(mpatcn]⁻ (pH 4.1) complexes separated by picolinate aromatic protons,
picolinate methylene protons, and acetate methylene protons. [Sc(mpatcn)] chemical
shifts are reproduced from previous work (Vaughn et al. 2020).
Chemical shift (ppm)

| | δ (¹H) Picolinate | δ (¹H) picolinate | δ (¹H) acetate | |
|---|---|---|---|---|
| | aromatic | methylene | methylene | Azamacrocycle |
| H₃mpatcn | 8.21 (t, 1H, H¹) | 4.34 (s, 2H, H⁴) | 3.83 (s, 4H, H⁸) | 3.16-3.74 (m, 12H, |
| | 8.10 (d, 1H, H²) | | | H⁵, H⁶, H⁷) |
| | 7.85 (d, 1H, H³) | | | |
| [Sc(mpatcn)] | 8.13 (t, 1H, H¹) | 4.43 (s, 2H, H⁴) | 3.78 (m, 2H, H⁸) | 2.95-3.24(m, 12H, |
| | 7.97 (d, 1H, H²) | | 3.42 (m, 2H, H⁸) | H⁵, H⁶, H⁷) |
| | 7.61 (d, 1H, H³) | | | |
| [ScF(mpatcn)]⁻ | 8.15 (t, 1H, H¹) | 3.87, 3.36 | 3.54, 3.34 | 2.90-3.29 |
| | 8.03 (d, 1H, H²) | (dd, H⁴)* | (dd, H⁸)* | (m, H⁵, H⁶, H⁷)* |
| | 7.63 (d, 1H, H³) | | | |

*Non-quantitative integration is expected to be a result of the H₂O suppression method.

TABLE 7
¹⁹F and ⁴⁵Sc chemical shift analysis of internal chemical shift standards,
[Sc(mpatcn)] (pH 4-6) and [ScF(mpatcn)]⁻ (pH 4.1) complexes.
[Sc(mpatcn)] chemical shifts are reproduced from previous work.¹
Chemical shift (ppm)

| | δ (¹⁹F) | δ (⁴⁵Sc) | |
|---|---|---|---|
| TFA/ScCl₃ | −75.5 ppm | −0.45 | ppm |
| [Sc(mpatcn)] | N/A | 80.1 | ppm |
| ScF(mpatcn)]⁻ | −15.5 ppm, br | 61.5 | ppm |

### 3. Synthesis and Characterization of Radiochemical Complexes

### 3.1 Spectrophotometric Titrations: Ligand Stock Concentration Determination

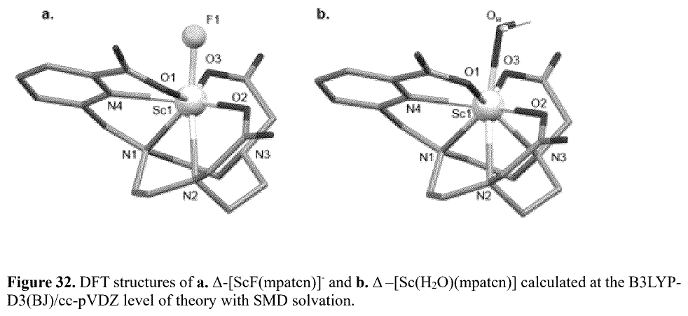

To determine the concentration of the picaga-DUPA and Hampaten samples used for radiolabeling experiments, spectrophotometric titrations were carried out with Cu²⁺. The formation of [Cu (mpaten)] or [Cu(picaga)-DUPA] was monitored at 300 nm using a 1 cm path length cuvette and a NanoDrop spectrophotometer. The pH was adjusted to 5.5 using 10 mM sodium acetate buffer. For H₃mpaten, a 1.67 mM ligand stock solution (100.6 μL) was titrated with addition of 10 μL (9.9 nmol) Cu²⁺ aliquots (as determined by ICP-OES) to determine the concentration of ligand by equivalents of Cu²⁺. Due to limited sample availability, a 0.16 mM picaga-DUPA stock solution (97.3 μL) was titrated with addition of 10 μL (0.98 nmol) Cu²⁺ aliquots. The titration endpoint was determined when no further change to the absorbance intensity at 300 nm, diagnostic of complex formation, was detected. Different batches of H₃mpaten were used for the various radiolabeling experiments. The concentration of each batch was determined using this method, and a representative titration is shown below. Analysis of the H₃mpaten and picaga-DUPA samples reveals 49.7% and 79.8% w/w content of ligand in TFA salt following deprotection, respectively. (See FIG. 32).

### 3.2 [¹⁸F]NaF Processing Protocol

[¹⁸F]NaF was received from NCM USA (Bronx, NY) in 50 mCi batches. A Sep-Pak Waters QME cartridge was primed with 9 mL water, the received [¹⁸F]NaF solution was loaded onto the cartridge, washed with 9 mL water, then eluted with 500 μL 0.4 M potassium bicarbonate. The eluate was adjusted to pH 2.5 with 1M HCl to afford a [¹⁸F]KF stock solution (total volume 638-693 μL).

### 3.3 General Radiolabeling Protocol

Inspired by previous success with pre-forming the [Al-¹⁸F]²⁺ complex prior to ligand addition, this approach was applied to [Sc-¹⁸F]²⁺ precursor (McBride et al. 2009). To do so, 0.615-1.023 [¹⁸F]KF stock was added to 200 μL 0.25 M ammonium acetate (pH 4.1) buffer, followed by addition of 0.2 equivalents of ScCl₃ relative to the amount of ligand added in the next step. Following incubation at room temperature for 10 minutes, the appropriate amount of ligand was added (5-800 nmol) and the mixture was incubated at the specified temperature (40-100° C.) for the specified duration (30-45 minutes) prior to radioHPLC analysis (Method C). Total reaction volumes ranged from 270-480 μL.

### 3.4 Representative Radiolabeling Traces of Mpaten

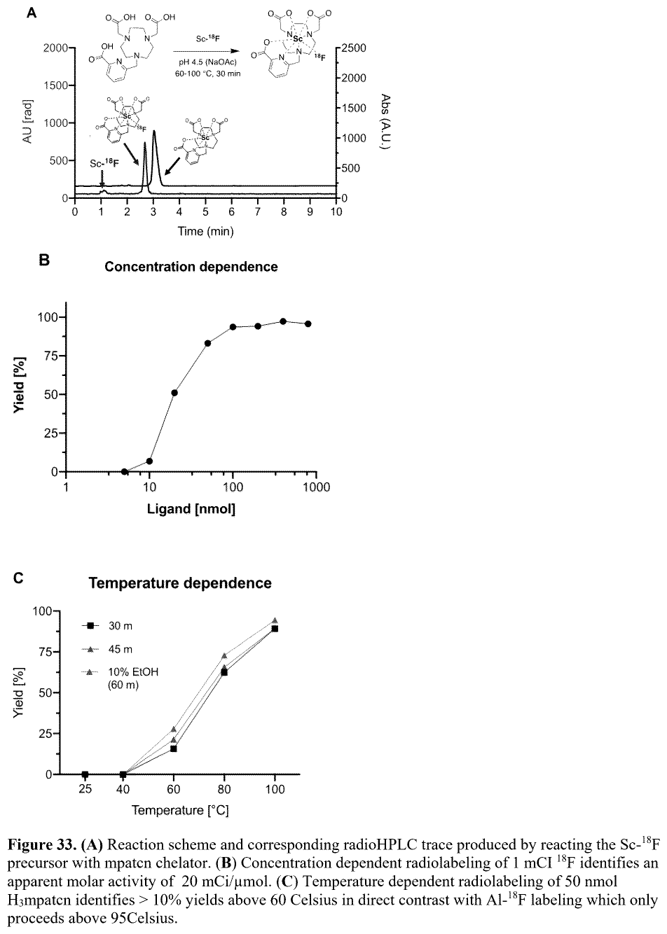

Concentration-dependent Radiolabeling was shown in FIG. 33(A) and Temperature-dependent Radiolabeling was shown in FIG. 33(B)

### 3.4.3 Optimized Radiolabeling Conditions

Based on temperature- and concentration-dependent radiolabeling with mpaten, optimized radiolabeling conditions were determined. Incubate 10 nmol ScCl₃ (0.2 eq.) with desired amount of [¹⁸F]KF stock at room temperature for 10 minutes in 0.25 M ammonium acetate (pH 4.1) buffer. Add 50 nmol ligand (1 eq.), then heat for 30 minutes at 60-100° C. Purify via radioHPLC (Method C), concentrate to dryness under reduced pressure, and formulate to desired volume in DPBS. Total time to formulation: 70 minutes. Rapid alternative purification methods are under investigation to further reduce this time.

### 4. In Vivo Imaging and Biodistribution

### 4.1 Synthesis and Purification of [¹⁸F]ScF(Picaga)-DUPA

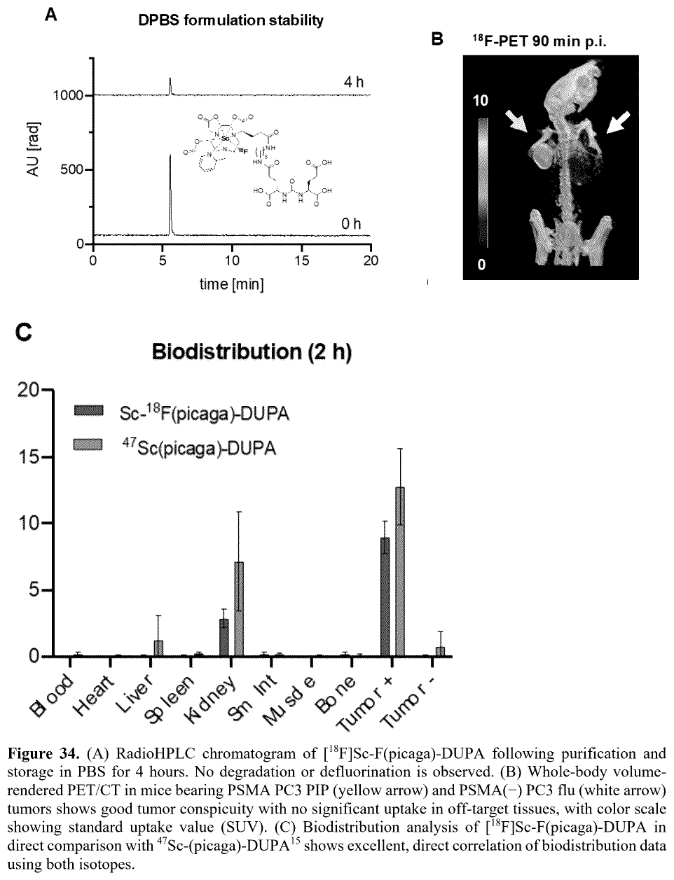

The [¹⁸F]KF stock solution was prepared as previously described (39.4 mCi, 693 μL, pH 2.5). The Sc-¹⁸F precursor was formed via direct addition of 200 μL 0.25 M ammonium acetate (pH 4.1) buffer and 20 nmol ScCl₃ stock (0.25 eq.) to the [¹⁸F]KF stock. Following incubation at room temperature for 10 minutes, 79.8 nmol picaga-DUPA (1 eq.) was added to 400 μL of the Sc-¹⁸F precursor stock solution (13.11 mCi), then incubated at 100° C. for 25 minutes. The major [¹⁸F]Sc—F(picaga)-DUPA species was separated the minor isomer and Sc-¹⁸F via analytical radioHPLC (Method C). (FIG. 34) Purified [¹⁸F]Sc—F(picaga)-DUPA complex was concentrated to dryness under reduced pressure and formulated for injection in DPBS (2.11 mCi, 550 μL). Dose formulation stability was analyzed at 0 and 4 hours (FIG. 34A), before and after in vivo experiments, via radioHPLC (Method C). Apparent molar activity: 164 mCi/μmol.

### 4.2 ¹⁸F-PET/CT Imaging and Biodistribution

All animal experiments and procedures were performed in accordance with the National Institute of Health's “Guide for the Care and Use of Laboratory Animals” and approved by Institutional Animal Care and Use Committee (IACUC) at Stony Brook Medicine.

Eight-week-old male mice were inoculated subcutaneously on the right shoulder with 1.0×10⁶ PSMA (+) PC3 PIP cells in 1:1 DPBS pH 7.4: Matrigel or on the left shoulder with 1.0×10⁶ PSMA (−) PC3 flu cells in 1:1 DPBS pH 7.4: Matrigel. At day 9 post-xenograft when tumors reached a suitable size, animals were administered [¹⁸F]Sc—F(picaga)-DUPA (206-273 μCi in 80 μL DPBS) via tail-vein injection. Mice were imaged at 90 min post injection (p.i.) using Siemens Inveon PET/CT Multimodality System, and image analysis was conducted using AMIDE. Upon completion of imaging at 120 min p.i., mice were sacrificed, select organs were harvested, and radioactivity was counted using a gamma counter. Counts per minute (CPM) values were decay corrected, and the radioactivity associated with each organ was expressed as % injected dose per gram (% ID/g). The processed data is plotted alongside previously published ⁴⁷Sc(picaga)-DUPA biodistribution data collected at 2 hours post-injection (n=4) for ease of direct comparison.

### 4.2.1 Tabulated Biodistribution Data

TABLE 8
Decay-corrected biodistribution of Sc-¹⁸F(picaga)-DUPA
and ⁴⁷Sc(picaga)-DUPA 2 h post-injection (n = 4).
⁴⁷Sc(picaga)-DUPA data is reproduced from previous
work (Vaughn et al. 2020)

| | Sc-¹⁸F(picaga)-DUPA | ⁴⁷Sc(picaga)-DUPA |
|---|---|---|
| Organ | 2 h (n = 4) | 2 h (n = 4) |
| Blood | 0.03 ± 0.01 | 0.19 ± 0.14 |
| Heart | 0.03 ± 0.01 | 0.11 ± 0.03 |
| Liver | 0.09 ± 0.01 | 1.22 ± 1.88 |
| Spleen | 0.1 ± 0.05 | 0.24 ± 0.10 |
| Kidney | 2.87 ± 0.69 | 7.13 ± 3.73 |
| Sm. Int. | 0.15 ± 0.20 | 0.18 ± 0.07 |
| Muscle | 0.03 ± 0.01 | 0.08 ± 0.03 |
| Bone | 0.17 ± 0.15 | 0.12 ± 0.06 |
| PSMA + Tumor | 8.96 ± 1.22 | 12.74 ± 2.87 |
| PSMA − Tumor | 0.09 ± 0.01 | 0.77 ± 1.11 |

### Density Functional Theory Calculations

### 5.1 Bond Lengths

TABLE 9
DFT determined bond lengths of isomers of [Sc(H₂O)(mpatcn)] and [ScF(mpatcn)]⁻

| Bond | Sc(H₂O)(mpatcn)Δ | Sc(H₂O)(mpatcn)Λ | ScF(mpatcn)]⁻Δ | [ScF(mpatcn)]⁻Λ |
|---|---|---|---|---|
| Sc1-O_{w} | 2.388 Å | 2.388 Å | — | — |
| Sc1-F1 | — | — | 1.959 Å | 1.959 Å |
| Sc1-O1 | 2.335 Å | 2.335 Å | 2.393 Å | 2.393 Å |
| Sc1-O2 | 2.115 Å | 2.115 Å | 2.139 Å | 2.139 Å |
| Sc1-O3 | 2.108 Å | 2.108 Å | 2.184 Å | 2.184 Å |
| Sc1-N1 | 2.484 Å | 2.484 Å | 2.540 Å | 2.540 Å |
| Sc1-N2 | 2.419 Å | 2.419 Å | 2.503 Å | 2.503 Å |
| Sc1-N3 | 2.554 Å | 2.554 Å | 2.637 Å | 2.637 Å |
| Sc1-N4 | 2.417 Å | 2.417 Å | 2.426 Å | 2.426 Å |

### 5.2 Bond Dissociation Energies

TABLE 10 (a)
[ScL(mpatcn)] and [AlL(N-benzyl-NODA)]⁺ BDE values, where
L = H₂O, OH⁻, or F⁻. All values are in kJ/mol.

| Complex | H₂O | OH⁻ | F⁻ |
|---|---|---|---|
| ScL(mpatcn) | 27.15 | 189.25 | 229.22 |
| [AlL(N-benzyl-NODA)]⁺ | 109.27 | 338.06 | 389.88 |

TABLE 10 (b)
Enthalpies of formation and corresponding
BDE's of discussed compounds

| | Sum of Electronic | | |
|---|---|---|---|
| | and Thermal | | |
| | Enthalpies | BDE | BDE |
| Compound | (Hartree) | (Hartree) | (kJ/mol) |
| F⁻ | −99.9153 | — | — |
| H₂O | −76.4075 | — | — |
| OH⁻ | −75.8785 | — | — |
| Sc(mpatcn) Λ | −2091.71 | — | — |
| Sc(mpatcn) Δ | −2091.71 | | |
| [ScF(mpatcn)]⁻ Λ | −2191.71 | 0.087306 | 229.2219 |
| [ScF(mptacn)]⁻ Δ | −2191.71 | 0.087306 | 229.2219 |
| Sc(H₂O)(mpatcn) Λ | −2168.13 | 0.01034 | 27.14767 |
| Sc(H₂O)(mptacn) Δ | −2168.13 | 0.01034 | 27.14767 |
| [Sc(OH)(mpatcn)]⁻ Λ | −2167.66 | 0.072081 | 189.2487 |
| [Sc(OH)(mptacn)]⁻ Δ | −2167.66 | 0.072095 | 189.2854 |
| [Al(NODA-benzyl)]⁺ Λ | −1369.1 | — | — |
| [Al(NODA-benzyl)]⁺ Δ | −1369.1 | — | — |
| AlF(NODA-benzyl) Λ | −1469.16 | 0.148496 | 389.8762 |
| AlF(NODA-benzyl) Δ | −1469.16 | 0.148496 | 389.8762 |
| [Al(H₂O)(NODA-benzyl)]⁺ Λ | −1445.55 | 0.041618 | 109.2681 |
| [Al(H₂O)(NODA-benzyl)]⁺ Δ | −1445.55 | 0.041622 | 109.2786 |
| Al(OH)(NODA-benzyl) Λ | −1445.1 | 0.128761 | 338.062 |
| Al(OH)(NODA-benzyl) Δ | −1445.1 | 0.128761 | 338.062 |

### 5.3 Atomic Coordinates of Discussed Structures

TABLE 11
Atomic Coordinates of Sc-mpatcn IsomersSc-mpatcn lambda

| Atomic Coordinates of Sc-mpatcn | | | | | | | |
|---|---|---|---|---|---|---|---|
| IsomersSc-mpatcn lambda | | | | Sc-mpatcn delta | | | |
| Sc | 0 | 0 | 0 | Sc | 0 | 0 | 0 |
| N | −0.47088 | 0.493893 | 2.289279 | N | 0.470882 | 0.493892 | 2.289279 |
| C | −1.91402 | 0.382537 | 2.666179 | C | 1.914021 | 0.382536 | 2.66618 |
| C | −2.84175 | 0.233692 | 1.461769 | C | 2.841745 | 0.233691 | 1.461769 |
| N | −2.28137 | −0.7344 | 0.492488 | N | 2.28137 | −0.7344 | 0.492488 |
| C | −2.36114 | −2.12152 | 1.037363 | C | 2.361144 | −2.12152 | 1.037362 |
| C | −1.07486 | −2.91057 | 0.872196 | C | 1.074854 | −2.91057 | 0.872195 |
| N | 0.11661 | −2.14614 | 1.316265 | N | −0.11661 | −2.14614 | 1.316265 |
| C | 0.108366 | −1.86347 | 2.784098 | C | −0.10837 | −1.86347 | 2.784098 |
| C | 0.386528 | −0.39882 | 3.100048 | C | −0.38653 | −0.39882 | 3.100048 |
| H | 0.242031 | −0.22228 | 4.181248 | H | −0.24203 | −0.22228 | 4.181248 |
| H | 1.430841 | −0.15177 | 2.861796 | H | −1.43084 | −0.15177 | 2.861796 |
| H | −0.85745 | −2.17217 | 3.201618 | H | 0.857445 | −2.17217 | 3.201617 |
| H | 0.869239 | −2.47771 | 3.288258 | H | −0.86924 | −2.47771 | 3.288258 |
| C | 1.335121 | −2.89864 | 0.949042 | C | −1.33512 | −2.89864 | 0.949041 |
| C | 2.501375 | −1.9701 | 0.788076 | C | −2.50138 | −1.9701 | 0.788076 |
| C | 3.818322 | −2.28165 | 1.1317 | C | −3.81832 | −2.28165 | 1.131699 |
| C | 4.825139 | −1.3578 | 0.84506 | C | −4.82514 | −1.3578 | 0.84506 |
| C | 4.496875 | −0.15881 | 0.206287 | C | −4.49688 | −0.1588 | 0.206287 |
| C | 3.160992 | 0.075663 | −0.10169 | C | −3.16099 | 0.075663 | −0.10169 |
| C | 2.666462 | 1.296793 | −0.83712 | N | −2.1908 | −0.80377 | 0.202563 |
| O | 3.458171 | 2.191016 | −1.16528 | C | −2.66646 | 1.296794 | −0.83712 |
| O | 1.395891 | 1.282927 | −1.07724 | O | −3.45817 | 2.191017 | −1.16528 |
| N | 2.190798 | −0.80377 | 0.202563 | O | −1.39589 | 1.282927 | −1.07723 |
| H | 5.249396 | 0.583923 | −0.05499 | H | −5.2494 | 0.583925 | −0.05499 |
| H | 5.860742 | −1.5744 | 1.112048 | H | −5.86074 | −1.5744 | 1.112048 |
| H | 4.042494 | −3.23253 | 1.615533 | H | −4.0425 | −3.23252 | 1.615533 |
| H | 1.161723 | −3.37267 | −0.02985 | H | −1.16172 | −3.37267 | −0.02985 |
| H | 1.553067 | −3.7008 | 1.671498 | H | −1.55307 | −3.7008 | 1.671497 |
| H | −1.16493 | −3.86269 | 1.426896 | H | 1.164929 | −3.86269 | 1.426896 |
| H | −0.91591 | −3.15666 | −0.18589 | H | 0.915913 | −3.15666 | −0.18589 |
| H | −2.6356 | −2.06492 | 2.097017 | H | 2.6356 | −2.06492 | 2.097017 |
| H | −3.17447 | −2.673 | 0.540217 | H | 3.17447 | −2.673 | 0.540217 |
| C | −2.93231 | −0.64425 | −0.82606 | C | 2.932309 | −0.64425 | −0.82606 |
| C | −1.99804 | −1.12677 | −1.93274 | C | 1.998035 | −1.12676 | −1.93274 |
| O | −2.43618 | −1.64488 | −2.96046 | O | 2.436178 | −1.64488 | −2.96046 |
| O | −0.72861 | −0.88992 | −1.71514 | O | 0.728613 | −0.88992 | −1.71514 |
| H | −3.88599 | −1.19434 | −0.86308 | H | 3.885994 | −1.19434 | −0.86308 |
| H | −3.13793 | 0.415983 | −1.04379 | H | 3.137933 | 0.415983 | −1.04379 |
| H | −3.83942 | −0.08674 | 1.812629 | H | 3.839418 | −0.08674 | 1.812629 |
| H | −2.95841 | 1.190543 | 0.944645 | H | 2.958405 | 1.190542 | 0.944646 |
| H | −2.02978 | −0.47158 | 3.342647 | H | 2.029776 | −0.47158 | 3.342647 |
| H | −2.21715 | 1.270866 | 3.241555 | H | 2.217145 | 1.270865 | 3.241556 |
| C | −0.02962 | 1.898914 | 2.35857 | C | 0.029623 | 1.898913 | 2.358571 |
| C | −0.62594 | 2.673544 | 1.184437 | C | 0.625945 | 2.673543 | 1.184438 |
| O | −0.8097 | 3.894103 | 1.255511 | O | 0.809697 | 3.894102 | 1.255512 |
| O | −0.87078 | 1.944015 | 0.139528 | O | 0.870777 | 1.944015 | 0.139529 |
| H | −0.28665 | 2.375273 | 3.31834 | H | 0.28665 | 2.375272 | 3.31834 |
| H | 1.066296 | 1.930715 | 2.237513 | H | −1.0663 | 1.930715 | 2.237513 |

TABLE 12
Atomic Coordinates of ScF-mpatcn Isomers

| ScF-mpatcn lambda | | | | ScF-mpatcn delta | | | |
|---|---|---|---|---|---|---|---|
| Sc | 0 | 0 | 0 | Sc | 0 | 0 | 0 |
| N | 0.530171 | 0.899328 | −2.27442 | N | −0.53017 | 0.899327 | −2.27442 |
| C | 1.916534 | 0.745675 | −2.78967 | C | −1.91654 | 0.745675 | −2.78967 |
| C | 2.912987 | 0.446042 | −1.67744 | C | −2.91299 | 0.446042 | −1.67744 |
| N | 2.397437 | −0.66708 | −0.87178 | N | −2.39744 | −0.66708 | −0.87178 |
| C | 2.426251 | −1.92284 | −1.66208 | C | −2.42625 | −1.92284 | −1.66208 |
| C | 1.148799 | −2.73863 | −1.56633 | C | −1.1488 | −2.73863 | −1.56633 |
| N | −0.0849 | −1.92207 | −1.65809 | N | 0.084896 | −1.92207 | −1.65809 |
| C | −0.2785 | −1.34525 | −3.03092 | C | 0.278496 | −1.34525 | −3.03092 |
| C | −0.46692 | 0.179253 | −3.089 | C | 0.466923 | 0.179253 | −3.089 |
| H | −0.43069 | 0.484345 | −4.15272 | H | 0.430692 | 0.484344 | −4.15272 |
| H | −1.45687 | 0.453956 | −2.70025 | H | 1.45687 | 0.453955 | −2.70025 |
| H | 0.578982 | −1.63781 | −3.65135 | H | −0.57898 | −1.63781 | −3.65135 |
| H | −1.16261 | −1.80207 | −3.50154 | H | 1.16261 | −1.80208 | −3.50154 |
| C | −1.20477 | −2.81544 | −1.2924 | C | 1.204771 | −2.81544 | −1.2924 |
| C | −2.44684 | −2.03138 | −1.00502 | C | 2.446837 | −2.03138 | −1.00502 |
| C | −3.73922 | −2.52346 | −1.21269 | C | 3.739219 | −2.52346 | −1.21269 |
| C | −4.82594 | −1.71234 | −0.88912 | C | 4.825934 | −1.71234 | −0.88912 |
| C | −4.59229 | −0.42035 | −0.40978 | C | 4.592284 | −0.42035 | −0.40978 |
| C | −3.27468 | 0.003358 | −0.26485 | C | 3.274682 | 0.003357 | −0.26485 |
| C | −2.89784 | 1.422419 | 0.087108 | N | 2.229394 | −0.80366 | −0.5189 |
| O | −3.77904 | 2.200974 | 0.509666 | C | 2.897841 | 1.422419 | 0.087108 |
| O | −1.67347 | 1.705524 | −0.12662 | O | 3.77904 | 2.200974 | 0.509666 |
| N | −2.2294 | −0.80366 | −0.5189 | O | 1.673469 | 1.705525 | −0.12662 |
| H | −5.40482 | 0.267151 | −0.17862 | H | 5.404823 | 0.267151 | −0.17862 |
| H | −5.84573 | −2.07274 | −1.03487 | H | 5.845732 | −2.07274 | −1.03487 |
| H | −3.87914 | −3.5221 | −1.6277 | H | 3.879134 | −3.5221 | −1.6277 |
| H | −0.91834 | −3.35438 | −0.37598 | H | 0.918336 | −3.35438 | −0.37598 |
| H | −1.39263 | −3.57267 | −2.07349 | H | 1.392625 | −3.57267 | −2.07349 |
| H | 1.160093 | −3.50962 | −2.35986 | H | −1.16009 | −3.50962 | −2.35986 |
| H | 1.109372 | −3.26087 | −0.60506 | H | −1.10937 | −3.26087 | −0.60506 |
| H | 2.629672 | −1.67664 | −2.71006 | H | −2.62967 | −1.67664 | −2.71006 |
| H | 3.26593 | −2.5595 | −1.33448 | H | −3.26593 | −2.5595 | −1.33448 |
| C | 3.084177 | −0.83682 | 0.40738 | C | −3.08418 | −0.83682 | 0.40738 |
| C | 2.225954 | −1.70033 | 1.331679 | C | −2.22596 | −1.70033 | 1.33168 |
| O | 2.758872 | −2.41327 | 2.1988 | O | −2.75887 | −2.41327 | 2.198801 |
| O | 0.95378 | −1.59543 | 1.145869 | O | −0.95378 | −1.59542 | 1.14587 |
| H | 4.090579 | −1.28023 | 0.301607 | H | −4.09058 | −1.28023 | 0.301607 |
| H | 3.191369 | 0.149605 | 0.887187 | H | −3.19137 | 0.149605 | 0.887187 |
| H | 3.898888 | 0.210911 | −2.12348 | H | −3.89889 | 0.210911 | −2.12348 |
| H | 3.042543 | 1.313713 | −1.0217 | H | −3.04254 | 1.313713 | −1.0217 |
| H | 1.931297 | −0.05678 | −3.53538 | H | −1.9313 | −0.05678 | −3.53538 |
| H | 2.223967 | 1.65986 | −3.32192 | H | −2.22397 | 1.659859 | −3.32192 |
| C | 0.256928 | 2.329054 | −2.08 | C | −0.25693 | 2.329054 | −2.08 |
| C | 0.846572 | 2.795069 | −0.7438 | C | −0.84657 | 2.795069 | −0.7438 |
| O | 1.096057 | 3.996814 | −0.56537 | O | −1.09606 | 3.996813 | −0.56537 |
| O | 1.025143 | 1.87151 | 0.149592 | O | −1.02514 | 1.87151 | 0.149592 |
| H | 0.660343 | 2.948022 | −2.89844 | H | −0.66035 | 2.948022 | −2.89844 |
| H | −0.82502 | 2.494015 | −2.02001 | H | 0.825023 | 2.494015 | −2.02001 |
| F | −0.64464 | 0.285526 | 1.82791 | F | 0.644641 | 0.285528 | 1.82791 |

TABLE 13
Atomic Coordinates of Sc(H₂O)-mpatcn Isomers

| Sc(H2O)-mpatcn lambda | | | | Sc(H2O)-mpatcn delta | | | |
|---|---|---|---|---|---|---|---|
| Sc | 0 | 0 | 0 | Sc | 0 | 0 | 0 |
| N | 0.372703 | 0.708343 | −2.28273 | N | −0.3727 | 0.708339 | −2.28273 |
| C | 1.756905 | 0.59084 | −2.82838 | C | −1.7569 | 0.590835 | −2.82838 |
| C | 2.799461 | 0.401064 | −1.7342 | C | −2.79946 | 0.401063 | −1.7342 |
| N | 2.332501 | −0.64774 | −0.81311 | N | −2.3325 | −0.64774 | −0.81311 |
| C | 2.38791 | −1.97301 | −1.48464 | C | −2.38791 | −1.97301 | −1.48464 |
| C | 1.141182 | −2.80935 | −1.26783 | C | −1.14119 | −2.80935 | −1.26783 |
| N | −0.11037 | −2.02866 | −1.42886 | N | 0.110363 | −2.02867 | −1.42886 |
| C | −0.34127 | −1.60894 | −2.85322 | C | 0.341262 | −1.60895 | −2.85322 |
| C | −0.59783 | −0.10859 | −3.04189 | C | 0.597832 | −0.1086 | −3.04189 |
| H | −0.56377 | 0.118981 | −4.1233 | H | 0.563773 | 0.118974 | −4.12329 |
| H | −1.59945 | 0.157523 | −2.67996 | H | 1.599454 | 0.157514 | −2.67995 |
| H | 0.522982 | −1.92204 | −3.45206 | H | −0.52299 | −1.92204 | −3.45206 |
| H | −1.20694 | −2.15093 | −3.2624 | H | 1.206936 | −2.15094 | −3.2624 |
| C | −1.21548 | −2.89031 | −0.9533 | C | 1.215481 | −2.89032 | −0.9533 |
| C | −2.46081 | −2.08368 | −0.76276 | C | 2.460809 | −2.08369 | −0.76276 |
| C | −3.75289 | −2.58924 | −0.92628 | C | 3.752887 | −2.58925 | −0.92628 |
| H | −3.89433 | −3.62432 | −1.23824 | C | 4.838597 | −1.74728 | −0.68679 |
| C | −4.8386 | −1.74727 | −0.68678 | C | 4.603918 | −0.42084 | −0.3158 |
| C | −4.60392 | −0.42083 | −0.3158 | C | 3.28613 | 0.006881 | −0.1892 |
| C | −3.28613 | 0.006886 | −0.18919 | N | 2.241792 | −0.81549 | −0.39123 |
| C | −2.90835 | 1.425232 | 0.152795 | C | 2.908355 | 1.425228 | 0.152792 |
| O | −3.80122 | 2.260758 | 0.384418 | O | 3.80122 | 2.260753 | 0.384415 |
| O | −1.6464 | 1.648554 | 0.16165 | O | 1.646398 | 1.64855 | 0.161652 |
| N | −2.24179 | −0.81548 | −0.39123 | H | 5.415147 | 0.28291 | −0.13511 |
| H | −5.41515 | 0.282916 | −0.13511 | H | 5.858613 | −2.11745 | −0.80188 |
| H | −5.85862 | −2.11744 | −0.80187 | H | 3.894328 | −3.62433 | −1.23824 |
| H | −0.92154 | −3.30821 | 0.022109 | H | 0.921533 | −3.30821 | 0.022113 |
| H | −1.39128 | −3.73698 | −1.63788 | H | 1.391273 | −3.73698 | −1.63787 |
| H | 1.154983 | −3.66499 | −1.96756 | H | −1.15499 | −3.665 | −1.96756 |
| H | 1.12992 | −3.21592 | −0.25059 | H | −1.12993 | −3.21592 | −0.25059 |
| H | 2.547374 | −1.81674 | −2.55741 | H | −2.54737 | −1.81674 | −2.55742 |
| H | 3.260769 | −2.54407 | −1.12757 | H | −3.26077 | −2.54407 | −1.12758 |
| C | 3.055 | −0.66832 | 0.461957 | C | −3.055 | −0.66832 | 0.461955 |
| C | 2.224619 | −1.40153 | 1.513109 | C | −2.22462 | −1.40153 | 1.513108 |
| O | 2.766406 | −1.97591 | 2.464716 | O | −2.76641 | −1.97591 | 2.464717 |
| O | 0.938798 | −1.33494 | 1.334605 | O | −0.9388 | −1.33494 | 1.334604 |
| H | 4.056132 | −1.1248 | 0.380591 | H | −4.05613 | −1.1248 | 0.380589 |
| H | 3.175661 | 0.367879 | 0.816289 | H | −3.17566 | 0.367878 | 0.816285 |
| H | 3.768265 | 0.135186 | −2.19666 | H | −3.76826 | 0.135188 | −2.19666 |
| H | 2.943646 | 1.322217 | −1.16166 | H | −2.94364 | 1.322217 | −1.16166 |
| H | 1.78651 | −0.25183 | −3.52752 | H | −1.78651 | −0.25184 | −3.52752 |
| H | 2.001182 | 1.486921 | −3.41942 | H | −2.00118 | 1.486914 | −3.41942 |
| C | 0.015561 | 2.133422 | −2.18026 | C | −0.01556 | 2.133418 | −2.18026 |
| C | 0.670583 | 2.733437 | −0.9313 | C | −0.67058 | 2.733435 | −0.9313 |
| O | 0.854556 | 3.953525 | −0.84481 | O | −0.85456 | 3.953523 | −0.84481 |
| O | 0.975469 | 1.876981 | −0.0016 | O | −0.97547 | 1.87698 | −0.0016 |
| H | 0.309245 | 2.703906 | −3.07601 | H | −0.30924 | 2.703901 | −3.07601 |
| H | −1.06998 | 2.229783 | −2.04709 | H | 1.069979 | 2.229777 | −2.04709 |
| O | −0.57102 | 0.577733 | 2.245503 | O | 0.571021 | 0.577731 | 2.245505 |
| H | −0.8507 | 1.503567 | 2.133712 | H | 0.850704 | 1.503564 | 2.133706 |
| H | 0.250755 | 0.609737 | 2.763558 | H | −0.25075 | 0.609738 | 2.763562 |

TABLE 14
Atomic Coordinates of Sc(OH)-mpatcn Isomers

| Sc(OH)-mpatcn delta | | | | Sc(OH)-mpatcn delta | | | |
|---|---|---|---|---|---|---|---|
| Sc | 0 | 0 | 0 | Sc | 0 | 0 | 0 |
| N | 0.295705 | 0.926796 | −2.34035 | N | −0.29354 | 0.925589 | −2.34143 |
| C | 1.670153 | 0.904378 | −2.90529 | C | −1.66775 | 0.904236 | −2.90737 |
| C | 2.736611 | 0.683707 | −1.83895 | C | −2.73531 | 0.684963 | −1.84192 |
| N | 2.357924 | −0.46346 | −1.0022 | N | −2.35854 | −0.46206 | −1.00426 |
| C | 2.440209 | −1.72395 | −1.78236 | C | −2.44082 | −1.72297 | −1.78363 |
| C | 1.236171 | −2.63189 | −1.59679 | C | −1.23743 | −2.63144 | −1.59635 |
| N | −0.04864 | −1.8966 | −1.64783 | N | 0.047856 | −1.89687 | −1.6475 |
| C | −0.34109 | −1.38633 | −3.02834 | C | 0.341273 | −1.3881 | −3.02837 |
| C | −0.65663 | 0.113131 | −3.11617 | C | 0.6581 | 0.111078 | −3.11716 |
| H | −0.66837 | 0.397477 | −4.18642 | H | 0.669695 | 0.394997 | −4.18751 |
| H | −1.65659 | 0.314776 | −2.71131 | H | 1.65832 | 0.31212 | −2.71268 |
| H | 0.515942 | −1.62475 | −3.67118 | H | −0.51576 | −1.62631 | −3.67125 |
| H | −1.19768 | −1.93281 | −3.4525 | H | 1.197512 | −1.93575 | −3.45172 |
| C | −1.09955 | −2.82538 | −1.1884 | C | 1.098192 | −2.82567 | −1.18681 |
| C | −2.39008 | −2.09968 | −0.9609 | C | 2.388994 | −2.10015 | −0.96007 |
| C | −3.63447 | −2.69335 | −1.18903 | C | 3.63315 | −2.69451 | −1.18769 |
| H | −3.68384 | −3.71656 | −1.56264 | C | 4.786977 | −1.95384 | −0.94091 |
| C | −4.788 | −1.9525 | −0.94133 | C | 4.657681 | −0.63453 | −0.50572 |
| C | −4.6582 | −0.6336 | −0.50502 | C | 3.380758 | −0.1071 | −0.3216 |
| C | −3.38104 | −0.1068 | −0.32069 | N | 2.265665 | −0.83856 | −0.51745 |
| C | −3.18336 | 1.345419 | 0.062985 | C | 3.183819 | 1.345262 | 0.061851 |
| O | −4.19847 | 2.028408 | 0.330414 | O | 4.199429 | 2.028006 | 0.328175 |
| O | −1.97777 | 1.751315 | 0.062671 | O | 1.978356 | 1.751427 | 0.062865 |
| N | −2.26632 | −0.83862 | −0.51702 | H | 5.522447 | 0.001365 | −0.32437 |
| H | −5.52275 | 0.002425 | −0.32307 | H | 5.773051 | −2.39138 | −1.10614 |
| H | −5.77425 | −2.38955 | −1.1068 | H | 3.682049 | −3.71814 | −1.56022 |
| H | −0.76769 | −3.26026 | −0.23234 | H | 0.766029 | −3.25925 | −0.23025 |
| H | −1.24461 | −3.65834 | −1.89833 | H | 1.242965 | −3.65953 | −1.89573 |
| H | 1.260478 | −3.42142 | −2.37132 | H | −1.26171 | −3.42182 | −2.37001 |
| H | 1.280954 | −3.12831 | −0.62193 | H | −1.28299 | −3.1267 | −0.62092 |
| H | 2.55235 | −1.47536 | −2.84358 | H | −2.55181 | −1.47509 | −2.84515 |
| H | 3.348158 | −2.2858 | −1.50497 | H | −3.34933 | −2.28418 | −1.50676 |
| C | 3.135508 | −0.5592 | 0.233212 | C | −3.13726 | −0.55626 | 0.230465 |
| C | 2.410419 | −1.46432 | 1.228341 | C | −2.41323 | −1.46004 | 1.227502 |
| O | 3.049376 | −2.10793 | 2.075556 | O | −3.05282 | −2.10165 | 2.075715 |
| O | 1.121937 | −1.46737 | 1.122343 | O | −1.12469 | −1.46401 | 1.121958 |
| H | 4.164039 | −0.9262 | 0.067527 | H | −4.1657 | −0.92339 | 0.06439 |
| H | 3.197561 | 0.44154 | 0.690898 | H | −3.19973 | 0.445026 | 0.686899 |
| H | 3.715931 | 0.526522 | −2.3311 | H | −3.7143 | 0.528543 | −2.33501 |
| H | 2.827879 | 1.561507 | −1.18992 | H | −2.82641 | 1.563166 | −1.1934 |
| H | 1.730459 | 0.116572 | −3.66477 | H | −1.72821 | 0.116273 | −3.66666 |
| H | 1.876005 | 1.849872 | −3.43276 | H | −1.87227 | 1.849746 | −3.43532 |
| C | −0.12238 | 2.318496 | −2.1339 | C | 0.125393 | 2.317142 | −2.13546 |
| C | 0.500333 | 2.864364 | −0.845 | C | −0.49774 | 2.864003 | −0.84716 |
| O | 0.660524 | 4.086057 | −0.70264 | O | −0.65686 | 4.085855 | −0.70523 |
| O | 0.801376 | 1.979961 | 0.054476 | O | −0.80044 | 1.980142 | 0.052419 |
| H | 0.143178 | 2.970307 | −2.98329 | H | −0.13939 | 2.968719 | −2.98528 |
| H | −1.20952 | 2.353418 | −1.98883 | H | 1.212463 | 2.351459 | −1.9899 |
| O | −0.66073 | 0.249747 | 1.897372 | O | 0.661272 | 0.249853 | 1.897144 |
| H | −1.3036 | 0.974798 | 1.82437 | H | 1.304783 | 0.974249 | 1.823446 |

TABLE 15
Atomic Coordinates of Al-NODA-Benzyl Isomers

| Al-NODA-Benzyl lambda | | | | Al-NODA-Benzyl delta | | | |
|---|---|---|---|---|---|---|---|
| Al | 0 | 0 | 0 | Al | 0 | 0 | 0 |
| N | 0.511269 | −1.44478 | 1.351333 | N | −0.51127 | −1.44478 | 1.351332 |
| C | 1.029611 | −0.83116 | 2.61455 | C | −1.02961 | −0.83116 | 2.614549 |
| C | 1.517226 | 0.590162 | 2.333647 | C | −1.51723 | 0.590162 | 2.333646 |
| N | 0.522803 | 1.295834 | 1.467668 | N | −0.5228 | 1.295834 | 1.467668 |
| C | −0.76755 | 1.597651 | 2.188307 | C | 0.767546 | 1.597651 | 2.188307 |
| C | −1.99245 | 1.279946 | 1.328086 | C | 1.992447 | 1.279946 | 1.328086 |
| N | −1.85392 | −0.07567 | 0.692333 | N | 1.853922 | −0.07567 | 0.692333 |
| C | −1.92825 | −1.17841 | 1.720093 | C | 1.928247 | −1.17841 | 1.720093 |
| C | −0.78704 | −2.16868 | 1.532151 | C | 0.787036 | −2.16868 | 1.532151 |
| H | −0.72723 | −2.85575 | 2.388589 | H | 0.727232 | −2.85575 | 2.388589 |
| H | −0.95573 | −2.76763 | 0.626545 | H | 0.955733 | −2.76763 | 0.626545 |
| H | −1.89622 | −0.72619 | 2.716911 | H | 1.896219 | −0.72619 | 2.716912 |
| H | −2.88751 | −1.70121 | 1.640759 | H | 2.887514 | −1.70121 | 1.64076 |
| C | −2.82152 | −0.28537 | −0.45449 | C | 2.82152 | −0.28537 | −0.45448 |
| C | −4.27922 | −0.24313 | −0.07544 | C | 4.279221 | −0.24313 | −0.07544 |
| C | −4.95895 | 0.981346 | 0.022729 | C | 4.958948 | 0.981346 | 0.022729 |
| C | −6.30832 | 1.01684 | 0.381195 | C | 6.308318 | 1.01684 | 0.381195 |
| C | −6.99808 | −0.17288 | 0.638645 | C | 6.998082 | −0.17288 | 0.638645 |
| H | −8.05384 | −0.14458 | 0.915762 | H | 8.053845 | −0.14458 | 0.915762 |
| C | −6.33467 | −1.3982 | 0.526565 | C | 6.334674 | −1.3982 | 0.526565 |
| C | −4.98393 | −1.43181 | 0.168714 | C | 4.983932 | −1.43181 | 0.168715 |
| H | −4.47099 | −2.3906 | 0.064956 | H | 4.470985 | −2.3906 | 0.064957 |
| H | −6.87039 | −2.33134 | 0.711929 | H | 6.870391 | −2.33134 | 0.711929 |
| H | −6.82534 | 1.975845 | 0.452612 | H | 6.825344 | 1.975845 | 0.452612 |
| H | −4.42905 | 1.911008 | −0.19359 | H | 4.429045 | 1.911008 | −0.19359 |
| H | −2.59287 | 0.495795 | −1.1917 | H | 2.592869 | 0.495794 | −1.1917 |
| H | −2.56682 | −1.26002 | −0.89094 | H | 2.566821 | −1.26002 | −0.89094 |
| H | −2.89502 | 1.318898 | 1.952273 | H | 2.895018 | 1.318898 | 1.952273 |
| H | −2.10441 | 2.010099 | 0.516663 | H | 2.104412 | 2.010099 | 0.516663 |
| H | −0.78971 | 2.657947 | 2.469793 | H | 0.789714 | 2.657948 | 2.469792 |
| H | −0.78635 | 1.016144 | 3.11536 | H | 0.786352 | 1.016144 | 3.11536 |
| C | 1.061881 | 2.495262 | 0.777728 | C | −1.06188 | 2.495262 | 0.777728 |
| C | 0.481939 | 2.599284 | −0.63031 | C | −0.48194 | 2.599284 | −0.63031 |
| O | 0.501226 | 3.643589 | −1.26009 | O | −0.50123 | 3.643589 | −1.26009 |
| O | 0.011709 | 1.456967 | −1.11444 | O | −0.01171 | 1.456967 | −1.11444 |
| H | 0.865907 | 3.415245 | 1.343332 | H | −0.86591 | 3.415245 | 1.343332 |
| H | 2.150621 | 2.382352 | 0.666038 | H | −2.15062 | 2.382352 | 0.666038 |
| H | 2.460493 | 0.562334 | 1.770314 | H | −2.46049 | 0.562334 | 1.770314 |
| H | 1.683719 | 1.137778 | 3.272456 | H | −1.68372 | 1.137778 | 3.272456 |
| H | 0.224989 | −0.83356 | 3.358472 | H | −0.22499 | −0.83356 | 3.358472 |
| H | 1.849219 | −1.43621 | 3.023468 | H | −1.84922 | −1.43621 | 3.023468 |
| C | 1.495959 | −2.30177 | 0.650318 | C | −1.49596 | −2.30177 | 0.650318 |
| C | 1.202332 | −2.28625 | −0.84658 | C | −1.20233 | −2.28625 | −0.84658 |
| O | 1.601169 | −3.15679 | −1.59963 | O | −1.60117 | −3.15679 | −1.59963 |
| O | 0.516967 | −1.22105 | −1.25601 | O | −0.51697 | −1.22105 | −1.25601 |
| H | 2.501306 | −1.87603 | 0.789645 | H | −2.50131 | −1.87603 | 0.789645 |
| H | 1.494026 | −3.32995 | 1.037501 | H | −1.49403 | −3.32995 | 1.037501 |

TABLE 16
Atomic Coordinates of AlF-NODA-Benzyl Isomers

| AlF-NODA-benzyl lambda | | | | AlF-NODA-benzyl delta | | | |
|---|---|---|---|---|---|---|---|
| Al | 0 | 0 | 0 | Al | 0 | 0 | 0 |
| F | −0.43315 | −0.89818 | −1.45113 | F | 0.433153 | −0.89818 | −1.45113 |
| N | 0.390011 | −1.67399 | 1.15787 | N | −0.39001 | −1.67399 | 1.15787 |
| C | 0.844808 | −1.27402 | 2.528284 | C | −0.84481 | −1.27402 | 2.528284 |
| C | 1.319985 | 0.180649 | 2.564588 | C | −1.31999 | 0.180649 | 2.564588 |
| N | 0.362413 | 1.023614 | 1.804043 | N | −0.36241 | 1.023614 | 1.804043 |
| C | −0.9213 | 1.197584 | 2.552057 | C | 0.921303 | 1.197584 | 2.552057 |
| C | −2.13365 | 1.01471 | 1.647641 | C | 2.133654 | 1.01471 | 1.647641 |
| N | −1.95923 | −0.17574 | 0.770713 | N | 1.95923 | −0.17574 | 0.770713 |
| C | −2.03113 | −1.45711 | 1.549884 | C | 2.03113 | −1.45711 | 1.549884 |
| C | −0.90055 | −2.41074 | 1.169385 | C | 0.90055 | −2.41074 | 1.169385 |
| H | −0.87427 | −3.26155 | 1.868157 | H | 0.874274 | −3.26155 | 1.868157 |
| H | −1.05631 | −2.79485 | 0.153659 | H | 1.056305 | −2.79485 | 0.153659 |
| H | −1.99567 | −1.21908 | 2.618501 | H | 1.995672 | −1.21908 | 2.618501 |
| H | −2.99359 | −1.95491 | 1.38142 | H | 2.993591 | −1.95491 | 1.38142 |
| C | −2.9447 | −0.15963 | −0.36776 | C | 2.944696 | −0.15963 | −0.36776 |
| C | −4.40026 | −0.19642 | 0.031459 | C | 4.400259 | −0.19642 | 0.031459 |
| C | −5.09153 | 0.980779 | 0.360839 | C | 5.091526 | 0.980779 | 0.360839 |
| C | −6.43729 | 0.938178 | 0.73342 | C | 6.437288 | 0.938178 | 0.73342 |
| C | −7.11639 | −0.28432 | 0.775753 | C | 7.116387 | −0.28432 | 0.775753 |
| H | −8.16887 | −0.31756 | 1.064991 | H | 8.168866 | −0.31756 | 1.064991 |
| C | −6.44433 | −1.4611 | 0.432786 | C | 6.44433 | −1.4611 | 0.432786 |
| C | −5.09776 | −1.41436 | 0.059959 | C | 5.097757 | −1.41436 | 0.059959 |
| H | −4.58119 | −2.3334 | −0.2262 | H | 4.581186 | −2.3334 | −0.2262 |
| H | −6.97076 | −2.41775 | 0.448111 | H | 6.970758 | −2.41775 | 0.448111 |
| H | −6.96013 | 1.863478 | 0.984626 | H | 6.960128 | 1.863478 | 0.984626 |
| H | −4.57266 | 1.94016 | 0.314533 | H | 4.572656 | 1.94016 | 0.314533 |
| H | −2.73175 | 0.75216 | −0.94098 | H | 2.73175 | 0.75216 | −0.94098 |
| H | −2.69865 | −1.01925 | −0.99862 | H | 2.698654 | −1.01925 | −0.99862 |
| H | −3.03812 | 0.926415 | 2.267374 | H | 3.03812 | 0.926415 | 2.267374 |
| H | −2.25882 | 1.883986 | 0.990098 | H | 2.258816 | 1.883986 | 0.990098 |
| H | −0.95634 | 2.19505 | 3.012364 | H | 0.956336 | 2.19505 | 3.012364 |
| H | −0.94244 | 0.471515 | 3.372597 | H | 0.942444 | 0.471515 | 3.372597 |
| C | 0.916625 | 2.317581 | 1.35373 | C | −0.91662 | 2.317581 | 1.35373 |
| C | 0.280539 | 2.731218 | 0.026661 | C | −0.28054 | 2.731218 | 0.026661 |
| O | 0.278477 | 3.903523 | −0.33851 | O | −0.27848 | 3.903523 | −0.33851 |
| O | −0.20754 | 1.740042 | −0.68098 | O | 0.207535 | 1.740042 | −0.68098 |
| H | 0.788342 | 3.111465 | 2.103076 | H | −0.78834 | 3.111465 | 2.103076 |
| H | 1.99269 | 2.194076 | 1.162145 | H | −1.99269 | 2.194076 | 1.162145 |
| H | 2.302358 | 0.283803 | 2.091481 | H | −2.30236 | 0.283803 | 2.091481 |
| H | 1.406111 | 0.517905 | 3.609241 | H | −1.40611 | 0.517906 | 3.609241 |
| H | 0.013967 | −1.42444 | 3.225782 | H | −0.01397 | −1.42444 | 3.225782 |
| H | 1.658673 | −1.93456 | 2.857889 | H | −1.65867 | −1.93456 | 2.857889 |
| C | 1.475907 | −2.34642 | 0.412719 | C | −1.47591 | −2.34642 | 0.412719 |
| C | 2.418268 | −1.29522 | −0.17906 | C | −2.41827 | −1.29522 | −0.17906 |
| O | 3.57614 | −1.58346 | −0.47996 | O | −3.57614 | −1.58346 | −0.47996 |
| O | 1.879107 | −0.11754 | −0.355 | O | −1.87911 | −0.11754 | −0.355 |
| H | 2.033894 | −3.05296 | 1.043164 | H | −2.03389 | −3.05296 | 1.043164 |
| H | 1.040119 | −2.90065 | −0.43071 | H | −1.04012 | −2.90065 | −0.43071 |

TABLE 17
Atomic Coordinates of Al(H₂O)-NODA-Benzyl Isomers

| Al(H2O)-NODA-Benzyl lambda | | | | Al(H2O)-NODA-Benzyl delta | | | |
|---|---|---|---|---|---|---|---|
| Al | 0 | 0 | 0 | Al | 0 | 0 | 0 |
| O | −0.35919 | −0.93496 | −1.68636 | O | 0.359363 | −0.93494 | −1.68643 |
| H | 0.381926 | −0.87982 | −2.31771 | H | −0.38199 | −0.88038 | −2.31751 |
| H | −1.15785 | −0.74301 | −2.20816 | H | 1.157721 | −0.74249 | −2.20851 |
| N | 0.44518 | −1.69705 | 1.078896 | N | −0.44485 | −1.69719 | 1.078855 |
| C | 0.877518 | −1.28766 | 2.462062 | C | −0.87725 | −1.28786 | 2.462006 |
| C | 1.282438 | 0.187157 | 2.522529 | C | −1.28246 | 0.18688 | 2.522493 |
| N | 0.287662 | 0.996756 | 1.768398 | N | −0.28788 | 0.996716 | 1.768344 |
| C | −1.02123 | 1.088349 | 2.492725 | C | 1.021002 | 1.088562 | 2.492666 |
| C | −2.19941 | 0.861087 | 1.55433 | C | 2.199251 | 0.8615 | 1.554296 |
| N | −1.95449 | −0.32104 | 0.670664 | N | 1.954532 | −0.32068 | 0.670631 |
| C | −1.98145 | −1.60281 | 1.459579 | C | 1.981762 | −1.60245 | 1.459527 |
| C | −0.80723 | −2.50155 | 1.088075 | C | 0.807719 | −2.50144 | 1.088027 |
| H | −0.73628 | −3.34526 | 1.79087 | H | 0.73695 | −3.34516 | 1.79082 |
| H | −0.93909 | −2.90285 | 0.074803 | H | 0.939656 | −2.90271 | 0.074759 |
| H | −1.96538 | −1.35661 | 2.5264 | H | 1.965659 | −1.35625 | 2.526347 |
| H | −2.92123 | −2.13801 | 1.283925 | H | 2.921649 | −2.13746 | 1.28385 |
| C | −2.95457 | −0.35604 | −0.46155 | C | 2.954545 | −0.35552 | −0.46163 |
| C | −4.40825 | −0.32553 | −0.05388 | C | 4.408259 | −0.3251 | −0.05404 |
| C | −5.07146 | 0.895528 | 0.147741 | C | 5.071458 | 0.895916 | 0.147902 |
| C | −6.41493 | 0.922476 | 0.528152 | C | 6.414953 | 0.922772 | 0.528244 |
| C | −7.11851 | −0.2736 | 0.705577 | C | 7.118564 | −0.27335 | 0.705254 |
| H | −8.16927 | −0.2526 | 1.001762 | H | 8.169346 | −0.25243 | 1.001372 |
| C | −6.47461 | −1.49483 | 0.486741 | C | 6.47468 | −1.49452 | 0.486082 |
| C | −5.13005 | −1.51882 | 0.10507 | C | 5.130094 | −1.51842 | 0.104487 |
| H | −4.63812 | −2.47499 | −0.08752 | H | 4.638169 | −2.47453 | −0.08835 |
| H | −7.02137 | −2.43241 | 0.605691 | H | 7.021464 | −2.43213 | 0.604715 |
| H | −6.91599 | 1.880592 | 0.680705 | H | 6.916012 | 1.880848 | 0.681064 |
| H | −4.53161 | 1.832438 | −0.00246 | H | 4.531594 | 1.832865 | −0.00199 |
| H | −2.72877 | 0.507517 | −1.10204 | H | 2.728699 | 0.508116 | −1.102 |
| H | −2.75937 | −1.28054 | −1.01927 | H | 2.759315 | −1.27994 | −1.01949 |
| H | −3.11342 | 0.719173 | 2.147218 | H | 3.113277 | 0.719742 | 2.147187 |
| H | −2.34978 | 1.7279 | 0.899947 | H | 2.349464 | 1.728321 | 0.89989 |
| H | −1.11736 | 2.074911 | 2.965739 | H | 1.116959 | 2.07513 | 2.965695 |
| H | −1.02085 | 0.347023 | 3.299064 | H | 1.020757 | 0.347232 | 3.299005 |
| C | 0.774767 | 2.328816 | 1.33915 | C | −0.77533 | 2.328649 | 1.33908 |
| C | 0.138646 | 2.715236 | 0.002862 | C | −0.13991 | 2.715072 | 0.00246 |
| O | 0.083778 | 3.881758 | −0.3646 | O | −0.08629 | 3.881506 | −0.36546 |
| O | −0.2933 | 1.696719 | −0.71472 | O | 0.292917 | 1.696717 | −0.71483 |
| H | 0.583422 | 3.102775 | 2.094695 | H | −0.58368 | 3.102765 | 2.09439 |
| H | 1.859999 | 2.269997 | 1.169244 | H | −1.86065 | 2.269753 | 1.169772 |
| H | 2.259937 | 0.345731 | 2.053338 | H | −2.26 | 0.345232 | 2.05333 |
| H | 1.347599 | 0.512762 | 3.571485 | H | −1.34765 | 0.512452 | 3.571455 |
| H | 0.053766 | −1.49209 | 3.153549 | H | −0.05347 | −1.49215 | 3.1535 |
| H | 1.722649 | −1.91218 | 2.780669 | H | −1.72228 | −1.91252 | 2.780629 |
| C | 1.566581 | −2.32596 | 0.343177 | C | −1.56614 | −2.32632 | 0.343166 |
| C | 2.45214 | −1.23456 | −0.25702 | C | −2.45186 | −1.23513 | −0.25719 |
| O | 3.623912 | −1.44523 | −0.54742 | O | −3.62348 | −1.44615 | −0.54796 |
| O | 1.83178 | −0.09293 | −0.46266 | O | −1.83182 | −0.09328 | −0.46253 |
| H | 2.153968 | −2.99766 | 0.983163 | H | −2.15346 | −2.99804 | 0.983199 |
| H | 1.16055 | −2.91166 | −0.49461 | H | −1.16003 | −2.91208 | −0.49455 |

TABLE 18
Atomic Coordinates of Al(OH)-NODA-Benzyl Isomers

| Al(OH)-NODA-Benzyl lambda | | | | Al(OH)-NODA-Benzyl delta | | | |
|---|---|---|---|---|---|---|---|
| Al | 0 | 0 | 0 | Al | 0 | 0 | 0 |
| O | −0.44092 | −0.88992 | −1.51201 | O | 0.440899 | −0.89003 | −1.51197 |
| H | 0.349183 | −0.99935 | −2.06073 | H | −0.34926 | −0.99946 | −2.06063 |
| N | 0.397725 | −1.66871 | 1.202485 | N | −0.39773 | −1.66872 | 1.202481 |
| C | 0.834432 | −1.26693 | 2.575705 | C | −0.83442 | −1.26693 | 2.575713 |
| C | 1.30939 | 0.185533 | 2.610839 | C | −1.30933 | 0.185546 | 2.610861 |
| N | 0.358851 | 1.023419 | 1.839318 | N | −0.35878 | 1.023432 | 1.83936 |
| C | −0.93152 | 1.189679 | 2.574752 | C | 0.931598 | 1.189652 | 2.574771 |
| C | −2.13603 | 1.004736 | 1.661614 | C | 2.1361 | 1.0047 | 1.661619 |
| N | −1.95925 | −0.18417 | 0.781896 | N | 1.959256 | −0.18418 | 0.781858 |
| C | −2.02926 | −1.4654 | 1.563271 | C | 2.029269 | −1.46544 | 1.563223 |
| C | −0.8857 | −2.41427 | 1.207859 | C | 0.885688 | −2.4143 | 1.207848 |
| H | −0.86297 | −3.25432 | 1.920281 | H | 0.862965 | −3.25433 | 1.920286 |
| H | −1.02796 | −2.81679 | 0.197895 | H | 1.027927 | −2.81682 | 0.197885 |
| H | −2.01426 | −1.22355 | 2.631624 | H | 2.014311 | −1.22359 | 2.631576 |
| H | −2.98444 | −1.97211 | 1.381417 | H | 2.984448 | −1.97214 | 1.381334 |
| C | −2.95774 | −0.16319 | −0.34511 | C | 2.957777 | −0.16319 | −0.34512 |
| C | −4.41029 | −0.19224 | 0.067794 | C | 4.410303 | −0.19226 | 0.067801 |
| C | −5.09537 | 0.989551 | 0.393788 | C | 5.095417 | 0.989532 | 0.393718 |
| C | −6.43838 | 0.954627 | 0.776992 | C | 6.43843 | 0.954602 | 0.776926 |
| C | −7.12142 | −0.26504 | 0.833642 | C | 7.121434 | −0.26508 | 0.833673 |
| H | −8.17169 | −0.29246 | 1.131333 | H | 8.171703 | −0.29251 | 1.131368 |
| C | −6.45615 | −1.44655 | 0.493604 | C | 6.456124 | −1.4466 | 0.493732 |
| C | −5.1124 | −1.4073 | 0.109853 | C | 5.112378 | −1.40734 | 0.10997 |
| H | −4.60207 | −2.33024 | −0.1749 | H | 4.602025 | −2.3303 | −0.17471 |
| H | −6.98587 | −2.40114 | 0.51956 | H | 6.98581 | −2.40121 | 0.519775 |
| H | −6.95587 | 1.883663 | 1.025496 | H | 6.955938 | 1.883644 | 1.025355 |
| H | −4.57377 | 1.946868 | 0.336393 | H | 4.573836 | 1.946857 | 0.336266 |
| H | −2.74645 | 0.747047 | −0.92108 | H | 2.746493 | 0.747066 | −0.92108 |
| H | −2.72652 | −1.02406 | −0.97917 | H | 2.726556 | −1.02404 | −0.97922 |
| H | −3.04381 | 0.913682 | 2.276084 | H | 3.043883 | 0.913583 | 2.276068 |
| H | −2.25951 | 1.875249 | 1.005485 | H | 2.259591 | 1.875227 | 1.00551 |
| H | −0.97687 | 2.185697 | 3.037852 | H | 0.976974 | 2.18566 | 3.037894 |
| H | −0.95826 | 0.461726 | 3.393546 | H | 0.958342 | 0.461697 | 3.393563 |
| C | 0.919899 | 2.320915 | 1.413536 | C | −0.91981 | 2.320936 | 1.413607 |
| C | 0.29775 | 2.754251 | 0.088388 | C | −0.29771 | 2.754271 | 0.088441 |
| O | 0.307028 | 3.932487 | −0.26253 | O | −0.30702 | 3.932492 | −0.2625 |
| O | −0.19022 | 1.775714 | −0.6316 | O | 0.190215 | 1.775718 | −0.63157 |
| H | 0.788972 | 3.104933 | 2.173336 | H | −0.78883 | 3.104952 | 2.173402 |
| H | 1.997158 | 2.196253 | 1.229222 | H | −1.99709 | 2.196315 | 1.229344 |
| H | 2.296539 | 0.287347 | 2.147486 | H | −2.29648 | 0.287387 | 2.147516 |
| H | 1.386552 | 0.527196 | 3.655203 | H | −1.3865 | 0.527183 | 3.655236 |
| H | −0.00605 | −1.4112 | 3.262823 | H | 0.006059 | −1.41124 | 3.262831 |
| H | 1.641915 | −1.92792 | 2.920761 | H | −1.64193 | −1.92788 | 2.920768 |
| C | 1.496947 | −2.33494 | 0.474441 | C | −1.49697 | −2.33495 | 0.474479 |
| C | 2.447086 | −1.27846 | −0.09744 | C | −2.44711 | −1.27845 | −0.09739 |
| O | 3.61622 | −1.5591 | −0.36297 | O | −3.61625 | −1.55909 | −0.36291 |
| O | 1.903356 | −0.10766 | −0.3001 | O | −1.90338 | −0.10765 | −0.30003 |
| H | 2.047321 | −3.0429 | 1.110376 | H | −2.04736 | −3.04288 | 1.110439 |
| H | 1.076991 | −2.88535 | −0.37933 | H | −1.07705 | −2.88541 | −0.37928 |

### Discussion

A solid phase synthesis approach was used for the construction of the trifunctional conjugate in lieu of the previously low yielding solution phase synthesis of picaga-DUPA (Scheme 1) (Umbricht, C. A. et al. 2018). This provided means to synthesize a more complex targeting scaffold than the first-generation molecule, while eliminating time-consuming, reverse-phase chromatography purification steps and significantly increasing over-all yields; an important aspect of relevance for scale-ups required for future clinical translation. The Glu-urea-Lys targeting moiety was synthesized according to a procedure previously described (Umbricht, C. A. et al. 2018). Similarly, PSMA-617 was also constructed on solid phase and synthesized as a reference compound of clinical relevance based on the approach previously reported (Eder, M. et al. 2012; Benesova, M. et al. 2015).

Picaga-HSA was synthesized from the resin-immobilized glu-urea-lys targeting moiety (Scheme 4). Dde-Lys (Fmoc) was activated with HBTU and coupled to the targeting moiety in the presence of diisopropylethyamine (DIPEA) and dimethylformamide (DMF). The Fmoc-protecting group was cleaved from the Dde-protected lysine in 20% piperidine in DMF. 4-(p-iodophenyl) butyric acid was coupled to the lysine via HBTU activation. Cleavage of the Dde-protecting group was accomplished in 2% hydrazine in DMF. The conjugation was performed with picaga by HBTU coupling in DMF. The tert-butyl protected chelator was cleaved from the resin in 1% TFA: DCM and then deprotected in 2:1 TFA: DCM overnight. Picaga-HSA was isolated following resin-cleavage in 0.6% overall non-optimized yield (0.0064 g) in 10 steps.

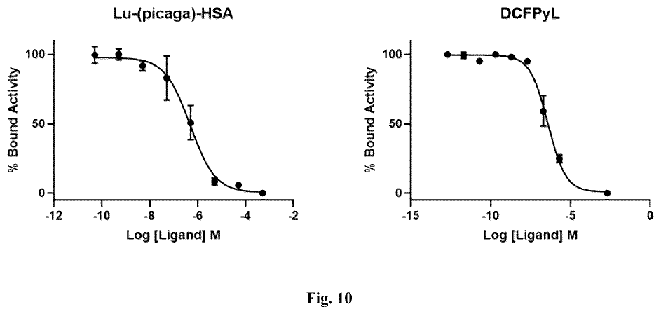

Prior to the assessment of the ¹⁷⁷Lu-labeled compounds, binding affinity to the biological target PSMA was evaluated. The affinity (Kᵢ) of ^{nat}Lu-(picaga)-HSA to the PSMA target was determined to be 1.4±0.6 nM by using a previously established displacement assay with the clinically investigated tracer ^{99m}Tc-MIP-1427 and non-radioactive, fluorinated small molecule DCFPyL as an internal reference (FIG. 10) (Vaughn, B. A. et al. 2020). The low nanomolar affinity of Lu-(picaga)-HSA indicates that neither the picaga chelator nor the HSA-binding moiety appear to adversely impact the affinity to PSMA when compared to PSMA-617 (Kᵢ=6.9±1.3 nM) (Benesova, M. et al. 2015).

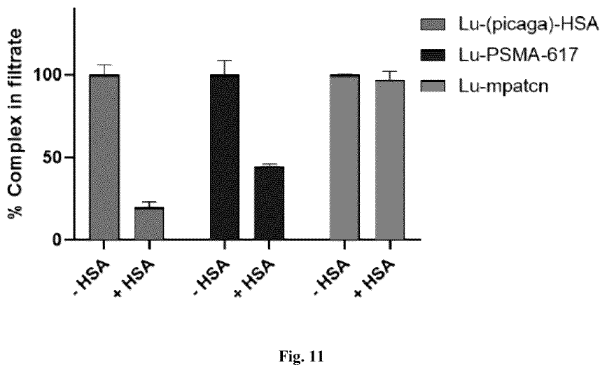

Interaction with ^{nat}Lu-(picaga)-HSA and ^{nat}Lu-PSMA-617 to human serum albumin was evaluated to affirm that the iodophenyl-butyrate significantly enhanced the binding to serum albumin. To this end, each ligand was incubated at 37° C. for 15 minutes in PBS with or without 4.5% human serum albumin to simulate in vivo protein concentration. Each mixture was filtered through a 50 KDa MW cutoff filter and the ^{nat}Lu content of the filtrate was measured by ICP-OES. ^{nat}Lu-mpaten was used as a chelator-only control with no targeting functionality and thus no expected interaction with human serum albumin. Lu-(picaga)-HSA had 80±3.3% binding to HSA compared to PBS while PSMA-617 had 55±1.2% binding compared to the PBS, in good agreement with literature reported values (Benesova, M. et al. 2018). There was no significant difference in the compound in the filtrate with or without HSA for Lu-mpaten (FIG. 11). The data indicates enhanced HSA interaction for ^{nat}Lu-(picaga)-HSA compared to ^{nat}Lu-PSMA-617, motivating further in vitro and in vivo studies.

Following synthesis and validation of picaga-HSA, radiolabeling with ¹⁷⁷Lu was carried out. Limited solubility in saline necessitated the addition of dimethylsulfoxide (DMSO) to solubilize the conjugate, a strategy commonly employed for other serum albumin targeting constructs (Umbricht, C. A. et al. 2018). As a consequence, the radiolabeling of picaga-HSA and PSMA-617 was performed in 30% DMSO. It was found that DMSO was necessary to solubilize the (picaga)-HSA ligand, but no precipitation or loss of labeled product was observed upon dilution of up to 0.5% DMSO in aqueous solution. At specific activities of (2.96 MBq/nmol), the construct labeled with a radiochemical yield (% RCY) of >99% as determined by radio-HPLC, requiring no further purification steps prior to dilution for injection (FIG. 12).

Formulation stability was monitored, and no degradation was observed in the dose formulation solution at 4 h, 24 h, or 48 h in contrast with conjugates that exhibit radiolytic degradation (Table 1). When analyzed by radio-HPLC after 14 days, ¹⁷⁷Lu-(picaga)-HSA was observed to be 74% intact. Under the same conditions, ¹⁷⁷Lu-PSMA-617 was determined to be 80% intact. This indicates that at the relevant concentrations and time points, no significant degradation occurs.

When compared to the first-generation construct ¹⁷⁷Lu-picaga-DUPA, the retention time of ¹⁷⁷Lu-picaga-HSA and ¹⁷⁷Lu-PSMA-617 indicated a significant difference in hydrophilicity as expected by the introduction of the HSA-binding moiety. Accordingly, the distribution coefficient in 1-octanol/PBS pH 7.4 (Log D_{7.4}) revealed values of −2.11±0.03 for ¹⁷⁷Lu-(picaga)-HSA and −2.71±0.08 for ¹⁷⁷Lu-PSMA-617 (Table 2).

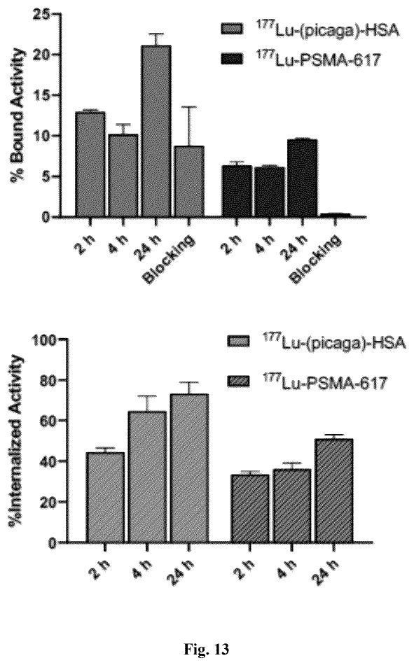

Cellular uptake and internalization of ¹⁷⁷Lu-picaga-HSA and ¹⁷⁷Lu-PSMA-617 was evaluated in PSMA±PC3 PIP cells (FIG. 13; Table 3). The bound activity ¹⁷⁷Lu-(picaga)-HSA was observed as 12.9=0.26% and did not increase after 4 h (10.2±1.18%). After 24 h, the observed binding increased significantly to 21.2±1.41%. The same trend was observed with ¹⁷⁷Lu-PSMA-617, albeit with overall lower binding observed (6.36±0.43, 6.15±0.21, 9.60±0.11% at 2, 4, and 24 hours respectively). However, blocking studies with unlabeled glu-urea-glu PSMA targeting molecule (Vaughn, B. A. et al. 2020) indicated that considerable non-specific binding of ¹⁷⁷Lu-(picaga)-HSA likely occurs (8.80±4.78 compared to 0.45±0.03 for ¹⁷⁷Lu-PSMA-617). Internalization of the ligand increased steadily over time for ¹⁷⁷Lu-(picaga)-HSA ranging from 44.5±2.00% at 2 h, 64.5±7.48% at 4 h and 73.26±5.72% at 24 h. By comparison internalization was lower for ¹⁷⁷Lu-PSMA-617, observed at 33.53±1.40%, 36.11±2.93%, and 50.93±2.14% at 2, 4, and 24 h respectively, it was hypothesized that this may be in part due to the enhanced lipophilic nature of ¹⁷⁷Lu-picaga-HSA as well as the compound's higher binding affinity to the PSMA target.

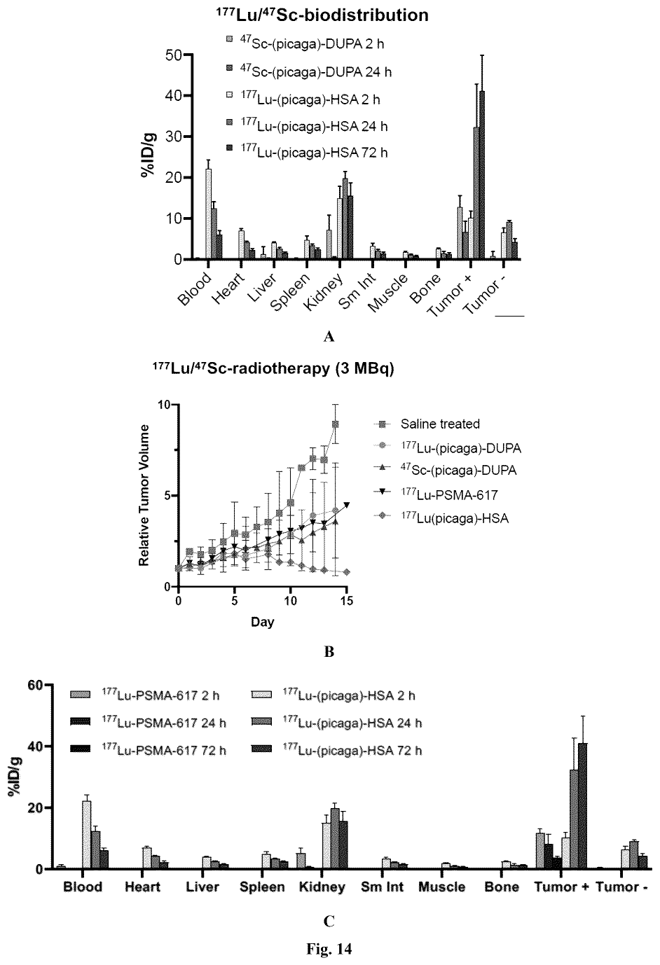

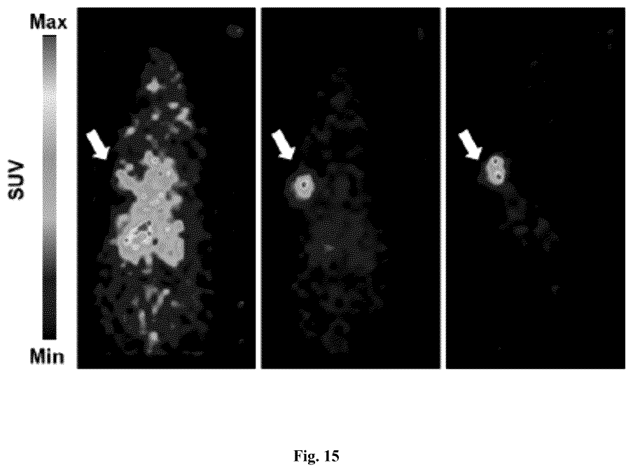

To assess the in vivo behavior of the ¹⁷⁷Lu-(picaga)-HSA and applicability to targeted therapy in comparison to ¹⁷⁷Lu-PSMA-617, a 1.7-2.2 MBq (45-60 μCi) dose of the radiolabeled compound at 4.1 MBq/nmol for ¹⁷⁷Lu-PSMA-617 and 2.0 MBq/nmol for ¹⁷⁷Lu-(picaga)-HSA specific activity was administered to mice bearing PSMA+ (PC-3 PiP) and PSMA-(PC-3 Flu) tumor xenografts on the right and the left flank, respectively. Cohorts were sacrificed at 2, 24, and 72 hours post injection for biodistribution analysis (FIG. 14; Table 4). These studies show that ¹⁷⁷Lu-(picaga)-HSA exhibits a distribution profile with slow blood clearance, low off-target uptake, and high PSMA+tumor uptake that continuously increased, from 10.2±1.47% ID/g at 2 h p.i. to 32.4±9.15 and 41.2±7.57% ID/g at 24 h and 72 h time points respectively. Low off-target uptake was observed, with 14.9±2.50% ID/g observed in the kidney at 2 h, 19.8±1.45 at 24 h, and 15.7±2.68 at 72 h. The activity observed in off-target organs such as muscle (1.79±0.22 to 0.82±0.08% ID/g at 2 h to 72 h respectively), liver (4.03±0.24 to 1.58±0.23% ID/g at 2 h to 72 h respectively) and spleen (4.83±0.79 to 2.46±0.35% ID/g at 2 h to 72 h respectively) can be attributed to human serum albumin interaction in the blood, as all organs except for the target tumor tissue show decrease in ¹⁷⁷Lu accumulation over time (FIG. 14; Table 4). The low uptake in the liver and small intestine indicates renal clearance. Free ¹⁷⁷Lu is shown to accumulate in bone (due to ionic similarities between ¹⁷⁷Lu³⁺ and Ca²⁺), as well as in the liver, spleen, and blood. Importantly, the minor uptake in the bone, which diminishes over time, is likely also caused by extended circulation in blood, as indicated by similar bone uptake data obtained with ¹⁷⁷Lu-PSMA-Alb-56 by Mueller and coworkers (Umbricht, C. A. et al. 2018). The clearance profile compares well with previously investigated DOTA conjugates, indicating that the seven-coordinate system of ¹⁷⁷Lu-(picaga)-DUPA is highly kinetically inert in vivo, which is desirable for constructs with comparatively slow clearance. Slow clearance and highly selective tumor accumulation are also observed in SPECT images of maximum-intensity projections (MIPs) of ¹⁷⁷Lu-(picaga)-HSA in PSMA+ tumor bearing mice up to 96 h (FIG. 15). At the same time, ¹⁷⁷Lu-PSMA-617 clears very rapidly and compares well to the behavior of first generation ¹⁷⁷Lu/⁴⁷Sc-picaga-DUPA constructs (Vaughn, B. A. et al. 2020).

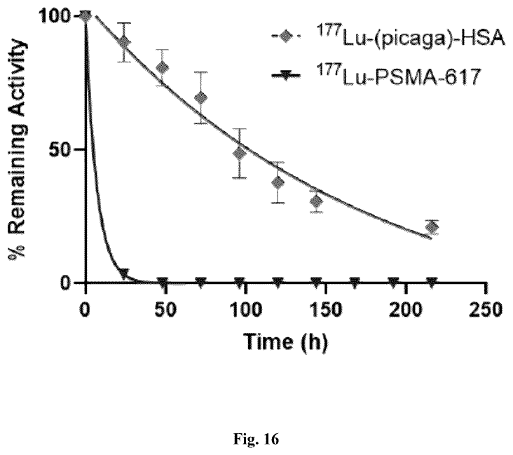

To approximate clearance of the ¹⁷⁷Lu-constructs, therapy cohort treated with ¹⁷⁷Lu-(picaga)-HSA and ¹⁷⁷Lu-PSMA-617 were monitored for residual whole-body activity remaining in the mouse. The whole-body activity remaining was plotted and the biological half-life in PSMA+tumor-bearing mice was determined using nonlinear regression in Graph Pad Prism (FIG. 16; Table 5). In mice bearing PSMA+ xenografts, significantly increased biological half-life was observed for ¹⁷⁷Lu-(picaga)-HAS (123 h) compared to ¹⁷⁷Lu-PSMA-617 (4.9 h). This is attributable to both the longer blood half-life because of the HSA-binding functionality and to the enhanced tumor uptake afforded by the longer blood circulation.

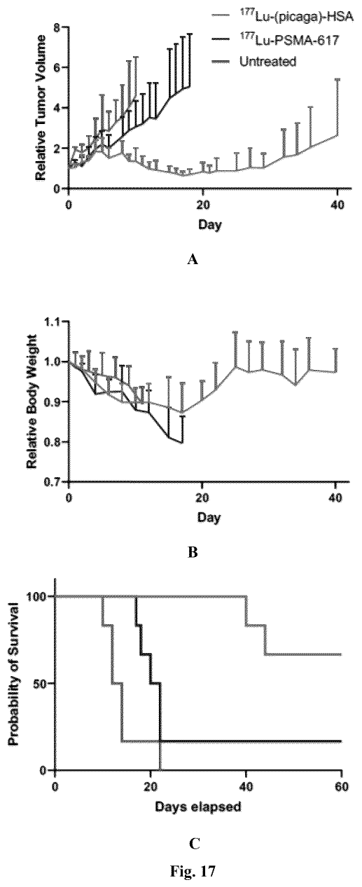

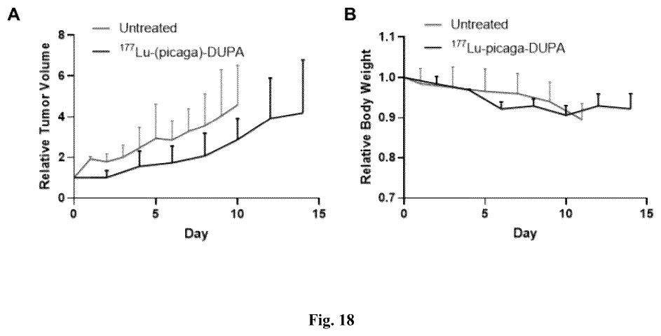

A single dose radiotherapy study was conducted to evaluate therapeutic efficacy of ¹⁷⁷Lu-(picaga)-HAS in a PSMA+ xenograft model in nude mice with a directly comparative study conducted with ¹⁷⁷Lu-PSMA-617 (FIG. 17). A single dose injection of saline, or 3.7 MBq of ¹⁷⁷Lu-(picaga)-HSA or 3.7 MBq of ¹⁷⁷Lu-PSMA-617 was administered at 5.9 MBq/nmol for ¹⁷⁷Lu-PSMA-617 and 3.0 MBq/nmol for specific activity ¹⁷⁷Lu-(picaga)-HSA. Mice treated with ¹⁷⁷Lu-PSMA-617 showed marginally enhanced survival and delayed tumor growth compared to the control cohort, with a median survival of 21 days (FIG. 17). Of note, the delay in tumor growth for ¹⁷⁷Lu-PSMA-617 was similar to the delay observed in our pilot data with ¹⁷⁷Lu-picaga-DUPA (FIG. 18). However, tumors in mice treated with ¹⁷⁷Lu-(picaga)-HSA showed significantly attenuated growth or even growth regression when compared to mice of the control cohort and mice treated with ¹⁷⁷Lu-PSMA-617 (FIG. 17). Four out of 6 mice survived to the termination of the study at day 60, and median survival was undefined (FIG. 17). Mice treated with ¹⁷⁷Lu-(picaga)-HSA demonstrated tumor regrowth after day 34. This observation is in accordance with similar, single dose radiotherapeutic treatment studies with ¹⁷⁷Lu-labeled small molecules (Umbricht, C. A. et al. 2018; Bandara, N. et al. 2018; Kuo, H.-T. et al. 2018). Interestingly, relative body weight of mice treated with 177Lu-PSMA-617 or vehicle/saline dropped during the study, while the body weight of mice treated with ¹⁷⁷Lu-(picaga)-HSA dropped initially but recovered, indicating that radiotoxicity and off-target effects were limited in severity (FIG. 17).

Because the highest observed off-target uptake was observed in the kidney, H&E staining of kidneys of surviving mice in the therapy cohorts was performed and compared with non-treated animals of the same age. No abnormal histopathological changes were observed for any mice (FIG. 19A-G) with the exception of mild perivascular inflammation along some blood vessels in one mouse treated with ¹⁷⁷Lu-picaga-HAS (FIG. 19C). The low therapeutic dose used to curb the otherwise rapid growth of the PC-3 xenografts demonstrates efficacy of ¹⁷⁷Lu-(picaga)-HSA, and importantly, the advantage in efficacy over ¹⁷⁷Lu-PSMA-617 at the studied dose level, indicates the suitability of ¹⁷⁷Lupicaga-HSA as radiotherapeutic drug for PSMA targeting.

The results described herein show that ¹⁷⁷Lu-(picaga)-HSA is a suitable candidate for further investigation toward translation for the management of prostate cancer using low dose radiotherapy and identifies picaga as a suitable chelator for theranostic applications.

Fluorine-18 is currently the most frequently and readily utilized PET isotope worldwide, for both diagnostic and research studies. The ease of production in large quantities using biomedical cyclotrons and automation of multi-step radiochemical syntheses with curie amounts of isotope has further streamlined and accelerated advancements in ¹⁸F-radiochemistry and de-novo tracer development, followed by subsequent preclinical and clinical translation (Fu et al. 2021 and Zheng et al. 2021). However, some shortcomings remain. Firstly, as conventional radiofluorination strategies involve C—F bond formation in anhydrous, aprotic solvents at high temperatures, the radiolabeling of thermally and chemically sensitive (bio) molecules remains inaccessible (Deng et al. 2019, Wright et al. 2021 and Sharninghausen et al. 2020). Previous attempts to address this issue involved the use of prosthetic group and the formation of B—F, Si—F and Al—F bonds. However, these strategies are hamstrung by production of low specific activity (B—F via isotope exchange reaction), incompatibility with aqueous solvent (Si—F) or high temperature reaction conditions and limited in vivo stability (Al—F) (Richter et al. 2015, Bernard-Gauthier et al. 2016, McBride et al. 2013 and Fersing et al. 2019). Secondly, as theranostic isotope pairs are becoming of imminent interest in nuclear medicine, the lack of a corresponding therapeutic partner to F-18 outlines a critical need (Hu et al. 2021). The Ga—F bond has been explored for direct radiofluorination, but while the Ga—F bond formation is feasible under forcing and low specific activity conditions, corresponding complexes are not sufficiently inert against defluorination in vitro and in vivo (Monzittu, et al. 2018, Koay et al. 2020, and Bhalla, et al. 2015). This is not surprising, considering the comparatively soft Lewis acid nature and kinetic lability of the Ga(III) ion. In contrast, Sc(III) exhibits greater chemical hardness and Lewis acidic character, thus it was considered as a viable alternative for the formation of ternary [¹⁸F][Sc(L)F] complexes sufficiently inert against radiodefluorination.

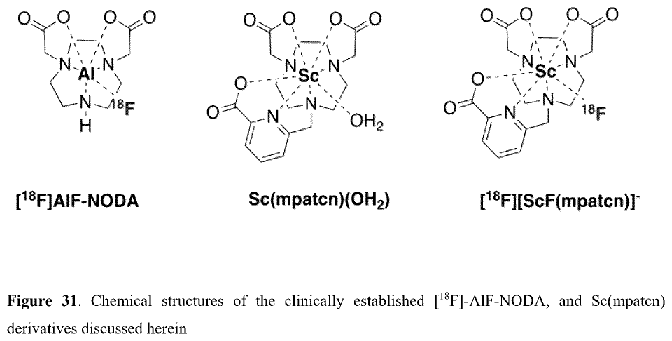

Previously, the chelation platform mpatcn/picaga (FIG. 31) was developed for radiometallic theranostic pairs, specifically ⁴⁴Sc (Eβ⁺_{avg}=632 keV, t_{1/2}=4h)/⁴⁷Sc (EB⁻_{avg}=162 keV, t_{1/2}=80.4 h) and the chemically homologous ¹⁷⁷Lu (EB⁻_{avg}=134 keV, t_{1/2}=159.6h) (Vaughn et al. 2021, Vaughn et al. 2020, and Nonat et al. 2009). Using extensive density functional, solution and gas-phase studies, a [Sc(mpatcn)(H₂O)] complex possesses an open coordination site was established, which binds an inner-sphere water molecule. Due to the high chemical hardness of the Sc(III) ion, the displacement of the labile ternary ligand H₂O by an inertly bound F⁻ could be feasible. Furthermore, in contrast to Al—F complexes, deprotonation of the Sc-bound inner sphere water molecule is unlikely to occur at radiochemically and physiologically relevant pH. The [Sc(mpatcn)(OH)]⁻ complex has a pKa of 9.1, while [(Al(NO₂A)(OH)] can form at much lower pH (<5); as a consequence, the displacement of F with OH represents a significant source of in vitro and in vivo defluorination of Al-¹⁸F complexes (Vaughn, 2020 et al. and D'Souza et al. 2011).

Here, a rapid, aqueous in situ formation of Sc-¹⁸F coordination complex [¹⁸F][ScF(mpatcn)]⁻ (FIG. 31) was introduced as means to access a high specific activity, one pot two-step labeling procedure for ¹⁸F. The formation of the Sc—F bond is exceptionally robust and in vivo compatible. A corresponding targeted PET agent can be prepared within shorter time and with higher specific activity than the FDA-approved C—F bond analogue [¹⁸F]DCFPyL and demonstrates ideal performance for the imaging of the prostate specific membrane antigen without indication of in vivo defluorination (Bouvet et al. 2016). The rapid and high-yielding formation of the Sc-¹⁸F bond will open possibilities to use ¹⁸F as part of a theranostic pair with ⁴⁷Sc and enables the preparation of ¹⁸F-containing radiopharmaceuticals without the need for anhydrous, cumbersome multi-step syntheses and purification or the use of organic co-solvents.

To evaluate the feasibility of the formation of and predict structural features of the [ScF (mpaten)] complex, a density functional theory calculation was employed (Frisch et al. 2009). Calculations were performed using the B3LYP(D3)-BJ functional with cc-PVDZ basis set and SMD solvation (Marenich et al., 2009). Complexes of mpaten were calculated starting from previously reported DFT structures (Vaughn et al. 2021). The [ScF(mpatcn)]⁻ complex showed very similar overall bonding structure and geometry when compared with the parent aqua complex [Sc(mpatcn)(H₂O)]. A significant lengthening of Sc—X bonds to all ligand donor atoms occurred by approximately 0.02-0.09 Å (Table 9). This is attributable to ionic repulsion caused by the negatively charged F and a significant reduction in the charge density at the metal center as compared to the weaker H₂O ligand. Structures of the delta isomers of the Sc—F and Sc—H₂O complexes of mpaten can be seen in FIG. 32.

To assess the strength of the Sc—F bond relative to Al—F, the bond dissociation enthalpy (BDE) of relevant scandium complexes of mpaten were computed and compared to ternary aluminum complexes of N-benzyl-NODA. This chelator was chosen as a benchmark, as the corresponding [¹⁸F][AlF(N-benzyl-NODA)] complex has excellent PBS and serum stability, as well as low ¹⁸F decomplexation in-vivo (D'Souza et al. 2011). A summary of the BDE of relevant complexes can be seen in Tables 10 (a) and 10 (b). While for L=H₂O, OH⁻, and F⁻, the Al-L BDE of the [Al(N-benzyl-NODA)]⁺ ternary complexes were higher than that of the corresponding Sc-L BDE of Sc(mpatcn) ternary complexes, it is important to note the competing species present in solution. The dominant species at physiological pH is the [Sc(mpatcn)(H₂O)] complex, with the corresponding hydroxide species forming only above pH 9 (Vaughn, et al. 2021 and Vaughn, et al. 2020). This is further evidenced by comparing BDE values for [Sc(mpatcn)(H₂O)]⁺ with [ScF(mpatcn)]⁻, with the Sc—F bond (229.22 kJ/mol) predicted to be nearly an order of magnitude stronger than the Sc—OH₂ bond (27.15 kJ/mol). However, in the case of the [Al(N-benzyl-NODA)]⁺ complex, the hydroxide species [Al(OH)(N-benzyl-NODA)] is the dominant species observed above pH 5.0, thus it represents the main competing species to the [AlF(N-benzyl-NODA)]⁺ complex. With BDEs only exhibiting a 10 kJ/mol difference between these species, this likely contributes to the need for high-temperature radiolabeling conditions and use of organic solvents for the complex [¹⁸F][AlF(N-benzyl-NODA)]⁺, as well as reversible displacement with the OH bound species as the main competing defluorination process.

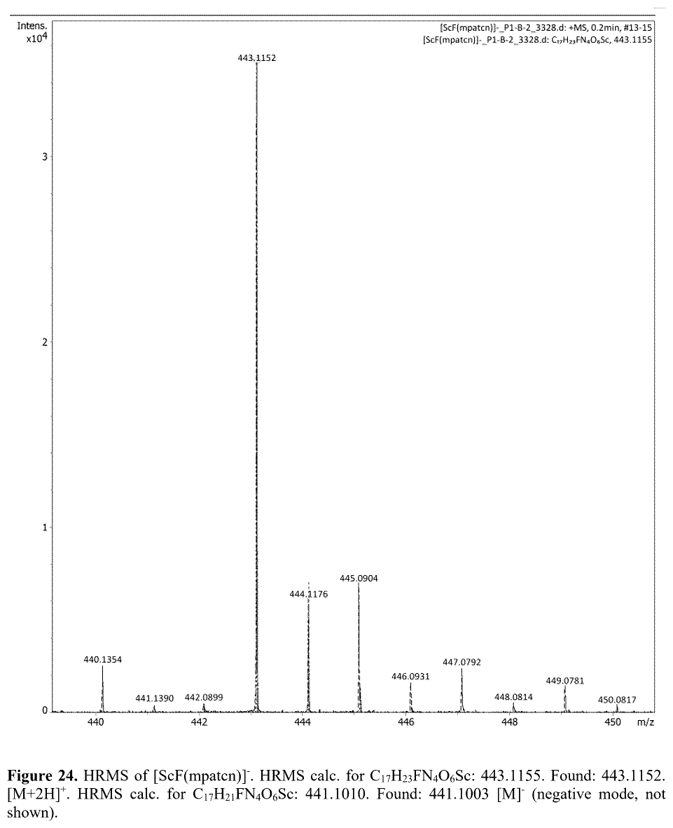

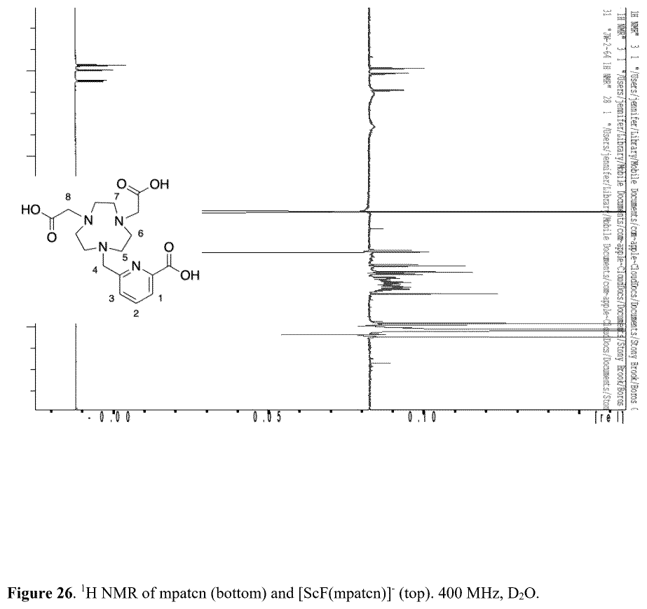

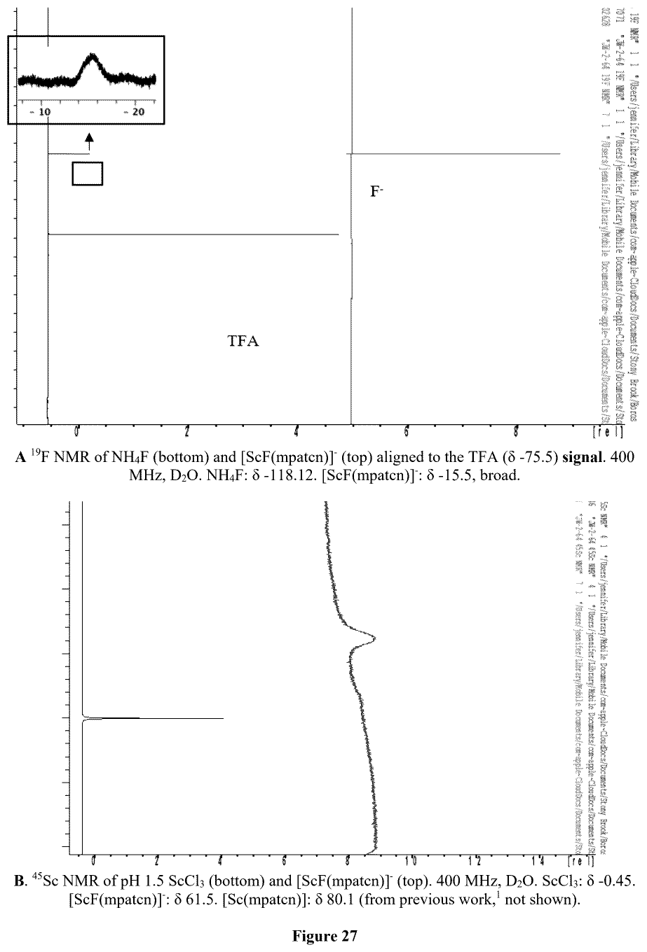

Supported by computational studies, [ScF(mpatcn)]⁻ on macroscopic scale was produced using direct fluorination of the open coordination site of [Sc(mpatcn)(H₂O)] with 5 equivalents of NH₄F, followed by isolation of the desired species using reverse phase chromatographic separation. In addition to conventional ¹H NMR, ¹⁹F and ⁴⁵Sc-NMR spectroscopy was utilized to characterize the [ScF(mpatcn)]⁻ ternary complex. FIGS. 26-27 show corresponding spectral data obtained for [ScF(mpatcn)]⁻. A more pronounced diastereotopic splitting pattern, especially for the macrocycle and methylene donor arm protons, combined with upfield shifts for the aromatic signals was observed in the ¹H spectrum, indicating enhanced rigidity. Both ¹⁹F and ⁴⁵Sc spectral data bear hallmarks of pronounced coupling, resulting in peak broadening of the 1° F. and ⁴⁵Sc signals. While the parent complex [Sc(mpatcn)(H₂O)] exhibits a ⁴⁵Sc chemical shift of δ=80.1 ppm, the signal for the [ScF (mpaten)] complex appears significantly broadened and shifted upfield at δ=61.5 ppm. The already anisotropic nature of the mpaten chelator leads to significant line broadening of the ⁴⁵Sc signal for the aqua complex due to the nuclear quadrupole; upon displacement of the aqua ion with F⁻ the signal further broadens indicating Sc—F coupling which could not be further resolved under these conditions. No exchange with bulk H₂O or formation of the corresponding OH bound species (δ=89.0 ppm) was observed. Similarly, ¹⁹F NMR showed a significantly broadened signal indicative of ¹⁹F-⁴⁵Sc coupling at δ=−15.5 ppm. This compares well to chemical shifts previously reported for more labile Lu—F and Y—F ternary complexes (Blackburn et al. 2015). Preceding literature spectroscopic data on Sc—F ternary complexes is scarce, but the observations match well with those reported (Curnock et al. 2018). Mass spectrometric analysis of the corresponding complex is straightforward and produces the expected complex species in positive and negative detection mode (FIG. 24).

With solution chemical studies affirming the formation and aqueous stability of the desired species, the current application validates possible methods for the formation of the corresponding radiofluorinated species. The initial attempts to fluorinate the pre-formed [Sc(mpaten)(H₂O)] complex in direct homology with the macroscopic fluorination strategy produced <5% product, possibly due to a lack of electrostatic interaction between the neutral aqua complex and the negatively charged F. In direct analogy with Al-¹⁸F chemistry, the in situ pre-formation of the [Sc-¹⁸F] species, followed immediately by the addition of the mpaten ligand produced the desired product at significantly increased radiochemical yields (>20%) at pH 4.5 and incubation at 60° C., without the need for any organic solvent additives. High radiofluorination yields are observed at 80° C. (62%) and 100° C. (89%) after incubation for 30 minutes.

Various aspects of these initial results indicated very significant differences to Al-¹⁸F radiochemistry; specifically, the formation of the desired radiochemical species at 60° C. using a macrocyclic chelator system, without the need for any organic solvent additive represent marked improvements; of note, the addition of ethanol to the radiolabeling reaction did not lead to significant changes in radiochemical yield. An apparent molar activity of 20 mCi/μmol was determined using high-temperature radiolabeling conditions, which is comparable to various targeted radiopharmaceuticals and motivated the validation of the Sc-¹⁸F approach in vivo as a logical next step.

A small-molecule peptide conjugate ^{44/47}Sc(picaga)-DUPA as a preclinically validated theranostic pair was previously developed. The picaga-DUPA ligand would also efficiently produce the desired Sc-¹⁸F-complex species to form Sc-¹⁸F-(picaga)-DUPA. Indeed, using previously optimized radiolabeling conditions using the non-functionalized mpaten chelator, the desired complex forms readily. The target compound subsequently isolated using HPLC and formulated for injection in phosphate-buffered saline (PBS). Analysis of the formulated, purified Sc-¹⁸F-(picaga)-DUPA product demonstrated no detectable decomposition even after 4 hours (FIG. 34A). To assess the viability of the Sc-¹⁸F complex to serve as an ideal surrogate for Al-¹⁸F and as a potential direct diagnostic partner for ⁴⁷Sc, Sc-¹⁸F-(picaga)-DUPA was administered to mice bearing PSMA+ and PSMA-xenografts at a specific activity of 164 mCi/μmol. Mice were imaged at 90 minutes post injection, followed by biodistribution at the 2-hour time point. FIG. 34B shows representative PET-CT maximum injection projection volume rendering and the corresponding biodistribution analysis (FIG. 34C). As indicated by a complete lack of ¹⁸F in bone, excellent tumor conspicuity followed by near exclusive renal clearance of the construct, the Sc-¹⁸F(mpatcn) complexes can be considered fully in vivo compatible and inert to defluorination. A direct comparison with biodistribution data of ⁴⁷Sc(picaga)-DUPA shows excellent agreement with respect to target and off-target tissue uptake (FIG. 34C), demonstrating that the Sc-¹⁸F-(mpaten) type ternary complex is a suitable diagnostic partner for the emerging ⁴⁷Sc therapy isotope.

In summary, it was shown for the first time that the formation of ternary ^{nat}sc-¹⁸F complexes is not only feasible, but that these complexes are inert towards defluorination in vivo. Conclusively, Sc-¹⁸F complexes are ideally suited as an alternative to conventional C-¹⁸F bond formation or the use of large, lipoliphilic prosthetic groups to incorporate ¹⁸F using time-intensive and low-yielding radiochemical approaches. Furthermore, the demonstrated biologically homologous behavior of Sc-¹⁸F-ternary complex when directly compared with the corresponding ⁴⁷Sc-complex renders the ¹⁸F/⁴⁷Sc isotope pair an unusual, yet fully viable theranostic couple with prospective clinical utility.

### REFERENCES

Anderson, C. J. & Ferdani, R. Copper-64 Radiopharmaceuticals for PET Imaging of Cancer: Advances in Preclinical and Clinical Research. Cancer Biotherapy and Radiopharmaceuticals. 2009, 24 (4), 379-393.
Bandara, N. et al. Novel Structural Modification Based on Evans Blue Dye to Improve Pharmacokinetics of a Somastostatin-Receptor-Based Theranostic Agent. Bioconjugate Chem. 2018, 29 (7), 2448-2454.
Benesova, M. et al. Preclinical Evaluation of a Tailor-Made DOTA-Conjugated PSMA Inhibitor with Optimized Linker Moiety for Imaging and Endoradiotherapy of Prostate Cancer. J. Nucl. Med. 2015, 56 (6), 914-20.
Benesova, M. et al. Preclinical Evaluation of a Tailor-Made DOTA-Conjugated PSMA Inhibitor with Optimized Linker Moiety for Imaging and Endoradiotherapy of Prostate Cancer. J. Nucl. Med. 2015, 56 (6), 914-20.
Benesova, M. et al. Albumin-Binding PSMA Ligands: Optimization of the Tissue Distribution Profile. Mol. Pharm. 2018, 15 (3), 934-946.
Bernard-Gauthier, V.; Bailey, J. J.; Liu, Z.; Wangler, B. r.; Wangler, C.; Jurkschat, K.; Perrin, D. M.; Schirrmacher, R., From unorthodox to established: The current status of 18F-trifluoroborate- and 18F-SiFA-based radiopharmaceuticals in PET nuclear imaging. Bioconjugate Chem. 2016, 27 (2), 267-279.
Bhalla, R.; Levason, W.; Luthra, S. K.; McRobbie, G.; Sanderson, G.; Reid, G., Radiofluorination of a pre-formed gallium (III) aza-macrocyclic complex: towards next-generation positron emission tomography (PET) imaging agents. Chem. Eur. J. 2015, 21 (12), 4688.
Chong, H. S.; Ma, X.; Le, T.; Kwamena, B.; Milenic, D. E.; Brady, E. D.; Song, H. A.; Brechbiel, M. W., Rational Design and Generation of a Bimodal Bifunctional Ligand for Antibody-Targeted Radiation Cancer Therapy. Journal of Medicinal Chemistry 2008, 51, 118-125.
Blackburn, O. A.; Kenwright, A. M.; Beer, P. D.; Faulkner, S., Axial fluoride binding by lanthanide DTMA complexes alters the local crystal field, resulting in dramatic spectroscopic changes. Dalton trans. 2015, 44 (45), 19509-19517.
Blackburn, O. A.; Chilton, N. F.; Keller, K.; Tait, C. E.; Myers, W. K.; McInnes, E. J.; Kenwright, A. M.; Beer, P. D.; Timmel, C. R.; Faulkner, S., Spectroscopic and crystal field consequences of fluoride binding by [Yb·DTMA]3+ in aqueous solution. Angew. Chem. Int. Ed. 2015, 54 (37), 10783-10786.
Bouvet, V.; Wuest, M.; Jans, H.-S.; Janzen, N.; Genady, A. R.; Valliant, J. F.; Benard, F.; Wuest, F., Automated synthesis of [18 F] DCFPyL via direct radiofluorination and validation in preclinical prostate cancer models. EJNMMI research 2016, 6 (1), 1-15.
Frisch, M. J. T., G. W.; Schlegel, H. B.; Scuseria, G. E.; Robb, M. A.; Cheeseman, J. R.; Scalmani, G.; Barone, V.; Mennucci, B.; Pe-tersson, G. A.; Nakatsuji, H.; Caricato, M.; Li, X.; Hratchian, H.; P.; Izmaylov, A. F. B., J.; Zheng, G.; Sonnenberg, J. L.; Hada, M.; Ehara, M.; Toyota, K.; Fukuda, R.; Hasegawa, J.; Ishida, M.; Nakajima, T.; Honda, Y.; Kitao, O.; Nakai, H.; Vreven, T.; Montgomery, J. A., Jr.; Peralta, J. E.; Ogliaro, F.; Bearpark, M.; Heyd, J. J.; Brothers, E.; Kudin, K. N.; Staroverov, V. N.; Kobayashi, R.; Normand, J.; Raghavachari, K.; Rendell, A. B., J. C.; Iyengar, S. S.; Tomasi, J.; Cossi, M.; Rega, N.; Millam, N. J.; Klene, M.; Knox, J. E.; Cross, J. B.; Bakken, V.; Adamo, C.; Jaramillo, J.; Gomperts, R.; Stratmann, R. E.; Yazyev, O.; Austin, A. J.; Cammi, R.; Pomelli, C.; Ochterski, J. W.; Martin, R. L.; Morokuma, K.; Zakrzewski, V. G.; Voth, G. A.; Salvador, P.; Dannenberg, J. J.; Dapprich, S.; Daniels, A. D.; Farkas, Ö.; Foresman, J. B.; Ortiz, J. V.; Cioslowski, J.; Fox, D. J. Gaussian 09, Revision D.01, G09, Revision D.01; Wallingford, CT, 2009.
Curnock, E.; Levason, W.; Light, M. E.; Luthra, S. K.; McRobbie, G.; Monzittu, F. M.; Reid, G.; Williams, R. N., Group 3 metal trihalide complexes with neutral N-donor ligands—exploring their affinity towards fluoride. Dalton Trans. 2018, 47 (17), 6059-6068.
Deng, X.; Rong, J.; Wang, L.; Vasdev, N.; Zhang, L.; Josephson, L.; Liang, S. H., Chemistry for positron emission tomography: recent advances in 11C—, 18F—, 13N—, and 15O-labeling reactions. Angew. Chem. Int. Ed. 2019, 58 (9), 2580-2605.
D'Souza, C. A.; McBride, W. J.; Sharkey, R. M.; Todaro, L. J.; Goldenberg, D. M., High-Yielding Aqueous 18F-Labeling of Peptides via A118F Chelation. Bioconj. Chem. 2011, 22 (9), 1793-1803.
Eder, M. et al. 68Ga-Complex Lipophilicity and the Targeting Property of a Urea-Based PSMA Inhibitor for PET Imaging. Bioconjugate Chem. 2012, 23 (4), 688-697.
Fersing, C.; Bouhlel, A.; Cantelli, C.; Garrigue, P.; Lisowski, V.; Guillet, B., A comprehensive review of non-covalent radiofluorination approaches using aluminum [18F]fluoride: Will [18F] AlF replace 68Ga for metal chelate labeling? Molecules 2019, 24 (16), 2866.
Fu, Y.; Helbert, H.; Simeth, N. A.; Crespi, S.; Spoelstra, G. B.; van Dijl, J. M.; van Oosten, M.; Nazario, L. R.; van der Born, D.; Luurtsema, G.; Szymanski, W.; Elsinga, P. H.; Feringa, B. L., Ultrafast Photoclick Reaction for Selective 18F-Positron Emission Tomography Tracer Synthesis in Flow. J. Am. Chem. Soc. 2021, 143 (27), 10041-10047.
Fuscaldi, L. L. et al. Standardization of the [⁶⁸Ga]Ga-PSMA-11 Radiolabeling Protocol in an Automatic Synthesis Module: Assessments for PET Imaging of Prostate Cancer. Pharmaceuticals. 2021, 14 (5), 385.
Ghosh, A. & Heston, W. D. Tumor target prostate specific membrane antigen (PSMA) and its regulation in prostate cancer. J. Cell. Biochem. 2004, 91 (3), 528-539.
Hillier, S. M. et al. 99mTc-labeled small-molecule inhibitors of prostate specific membrane antigen for molecular imaging of prostate cancer. J. Nucl. Med. 2013, 54 (8), 1369-1376.
Hu, A.; Aluicio-Sarduy, E.; Brown, V.; MacMillan, S. N.; Becker, K. V.; Barnhart, T. E.; Radchenko, V.; Ramogida, C. F.; Engle, J. W.; Wilson, J. J., Py-Macrodipa: A Janus Chelator Capable of Binding Medicinally Relevant Rare-Earth Radiometals of Disparate Sizes. J. Am. Chem. Soc. 2021, 143 (27), 10429-10440.
Koay, H.; Haskali, M. B.; Roselt, P. D.; White, J. M.; Donnelly, P. S., Gallium Fluoride Complexes with Acyclic Bispicolinic Ligands as Potential New Fluorine-18 Labelled Imaging Agents. Eur. J. Inorg. Chem. 2020, 2020 (35), 3378-3386.
Kuo, H.-T. et al. Enhancing Treatment Efficacy of ¹⁷⁷Lu-PSMA-617 with the Conjugation of an Albumin-Binding Motif: Preclinical Dosimetry and Endoradiotherapy Studies. Mol. Pharmaceutics 2018, 15 (11), 5183-5191.
Marenich, A. V.; Cramer, C. J.; Truhlar, D. G., Universal Solvation Model Based on Solute Electron Density and on a Continuum Model of the Solvent Defined by the Bulk Dielectric Constant and Atomic Surface Tensions. J. Phys. Chem. B 2009, 113 (18), 6378-6396.
McBride, W. J.; Sharkey, R. M.; Goldenberg, D. M., Radiofluorination using aluminum-fluoride (Al 18 F). EJNMMI res. 2013, 3 (1), 1-11.
McBride, W. J.; Sharkey, R. M.; Karacay, H.; D'Souza, C. A.; Rossi, E. A.; Laverman, P.; Chang, C. H.; Boerman, O. C.; Goldenberg, D. M., A novel method of 18F radiolabeling for PET. J Nucl Med 2009, 50 (6), 991-8.
Monzittu, F. M.; Khan, I.; Levason, W.; Luthra, S. K.; McRobbie, G.; Reid, G., Rapid Aqueous Late-Stage Radiolabelling of [GaF3 (BnMe2-tacn)] by 18F/19F Isotopic Exchange: Towards New PET Imaging Probes. Angew. Chem. Int. Ed. 2018, 57 (22), 6658-6661.
Nonat, A.; Giraud, M.; Gateau, C.; Fries, P. H.; Helm, L.; Mazzanti, M., Gadolinium (III) complexes of 1, 4, 7-triazacyclononane based picolinate ligands: simultaneous optimization of water exchange kinetics and electronic relaxation. Dalton Trans. 2009, (38), 8033-8046.
Nonat, A. M.; Gateau, C.; Fries, P. H.; Helm, L.; Mazzanti, M., New Bisaqua Picolinate-Based Gadolinium Complexes as MRI Contrast Agents with Substantial High-Field Relaxivities. Eur. J. Inorg. Chem. 2012, 2049-2061.
Parr, R. G.; Pearson, R. G., Absolute hardness: companion parameter to absolute electronegativity. J. Am. Chem. Soc. 1983, 105 (26), 7512-7516.
Price, E. W. et al. H₄octapa-trastuzumab: versatile acyclic chelate system for 111In and ¹⁷⁷Lu imaging and therapy. J. Am. Chem. Soc. 2013, 135 (34), 12707-12721.
Price, E. W. & Orvig, C. Matching chelators to radiometals for radiopharmaceuticals. Chem. Soc. Rev. 2014, 43 (1), 260-290.

Richter, S.; Wuest, M.; Bergman, C. N.; Way, J. D.; Krieger, S.; Rogers, B. E.; Wuest, F., Rerouting the metabolic pathway of 18F-labeled peptides: the influence of prosthetic groups. Bioconjugate Chem. 2015, 26 (2), 201-212.

Salaam, J.; Tabti, L.; Bahamyirou, S.; Lecointre, A.; Hernandez Alba, O.; Jeannin, O.; Camerel, F.; Cianferani, S.; Bentouhami, E.; Nonat, A. M.; Charbonniere, L. J., Formation of Mono- and Polynuclear Luminescent Lanthanide Complexes based on the Coordination of Preorganized Phosphonated Pyridines. Inorg Chem 2018, 57 (10), 6095-6106.

Schmitt, M. et al. Promising prospects for 44Sc—/47Sc-based theragnostics: application of 47Sc for radionuclide tumor therapy in mice. J. Nucl. Med. 2014, 55 (10), 1658-1664.

Sharninghausen, L. S.; Brooks, A. F.; Winton, W. P.; Makaravage, K. J.; Scott, P. J. H.; Sanford, M. S., NHC-Copper Mediated Ligand-Directed Radiofluorination of Aryl Halides. J. Am. Chem. Soc. 2020, 142 (16), 7362-7367.

Szabo, Z. et al. Initial Evaluation of [¹⁸F] DCFPyL for Prostate-Specific Membrane Antigen (PSMA)-Targeted PET Imaging of Prostate Cancer. Mol. Imaging Biol. 2015, 17 (4), 565-574.

Umbricht, C. A. et al. Preclinical Development of Novel PSMA-Targeting Radioligands: Modulation of Albumin-Binding Properties To Improve Prostate Cancer Therapy. Mol. Pharmaceutics 2018, 15 (6), 2297-2306.

Vaughn, B.; Koller, A.; Chen, Z.; Ahn, S. H.; Loveless, C.; Cingoranelli, S.; Yang, Y.; Cirri, A.; Johnson, C.; Lapi, S.; Chapman, K.; Boros, E., Enforcing Homologous Structural, Chemical and Biological Behavior of Sc and Lu Isotopes using Restrictive Chelate Architecture. Bioconjugate Chem. 2021, 32, 1232-1241.

Vaughn, B. A. et al. Chelation with a twist: a bifunctional chelator to enable room temperature radiolabeling and targeted PET imaging with scandium-44. Chem. Sci. 2020, 11 (2), 333-342.

Vaughn, B. A. et al. Homologous Structural, Chemical, and Biological Behavior of Sc and Lu Complexes of the Picaga Bifunctional Chelator: Toward Development of Matched Theranostic Pairs for Radiopharmaceutical Applications. Bioconjugate Chem. 2021, 32 (7), 1232-1241.

Wright, J. S.; Sharninghausen, L. S.; Preshlock, S.; Brooks, A. F.; Sanford, M. S.; Scott, P. J. H., Sequential Ir/Cu-Mediated Method for the Meta-Selective C—H Radiofluorination of (Hetero) Arenes. J. Am. Chem. Soc. 2021, 143 (18), 6915-6921.

Zheng, Q.; Xu, H.; Wang, H.; Du, W.-G. H.; Wang, N.; Xiong, H.; Gu, Y.; Noodleman, L.; Sharpless, K. B.; Yang, G.; Wu, P., Sulfur [¹⁸F]Fluoride Exchange Click Chemistry Enabled Ultrafast Late-Stage Radiosynthesis. J. Am. Chem. Soc. 2021, 143 (10), 3753-3763.

1. A compound having the structure: 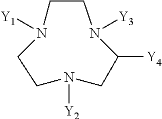
wherein
Y₁ and Y₂ are each, independently, —H, alkylheteroaryl, alkyl-CO₂H, alkylaryl-CO₂H, alkylheteroaryl-CO₂H, alkylheteroaryl-(NO₂)(CO₂H), alkyl-CO₂R₁, alkylaryl-CO₂R₁, alkylheteroaryl-CO₂R₁, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₁)₂, alkyl-N(alkylheteroaryl-CO₂R₁)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂, alkylheteroaryl-P(O)(OH)₂ or alkylheteroaryl-(NO₂)(P(O)(OH)₂),
wherein each occurrence of R₁ is, independently, —H, alkyl, alkenyl, alkynyl, alkyl-aryl, alkyl-heteroaryl, aryl, heteroaryl, alkyl-CF₃ or —Si(alkyl)₃; and
Y₃ and Y₄ are each, independently, —H, Z₁-L(A), Z₁-L(A)(B), L(A), L(A)(B), 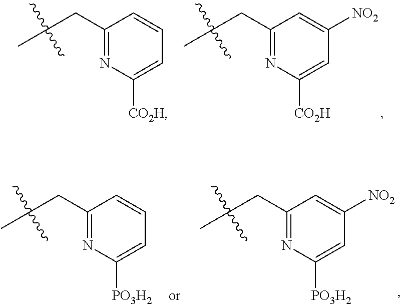
wherein
Z₁ is 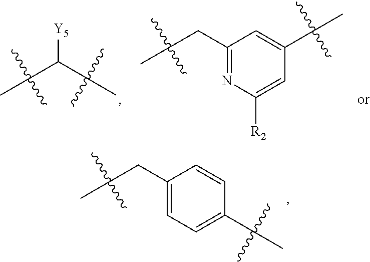
wherein
Y₅ is —CO₂H, —CO₂R₃, aryl-CO₂H, heteroaryl-CO₂H, aryl-CO₂R₃, heteroaryl-CO₂R₃, —P(O)(OH)₂, aryl-P(O)(OH)₂ or heteroaryl-P(O)(OH)₂,
wherein R₃ is, independently, —H, alkyl, alkenyl, alkynyl, alkyl-aryl, alkyl-heteroaryl, aryl, heteroaryl, alkyl-CF₃ or —Si(alkyl)₃,
R₂ is —CO₂H or —P(O)(OH)₂,
L is a chemical linker,
A is a targeting moiety, and
B is an albumin-binding moiety,
wherein the compound is not 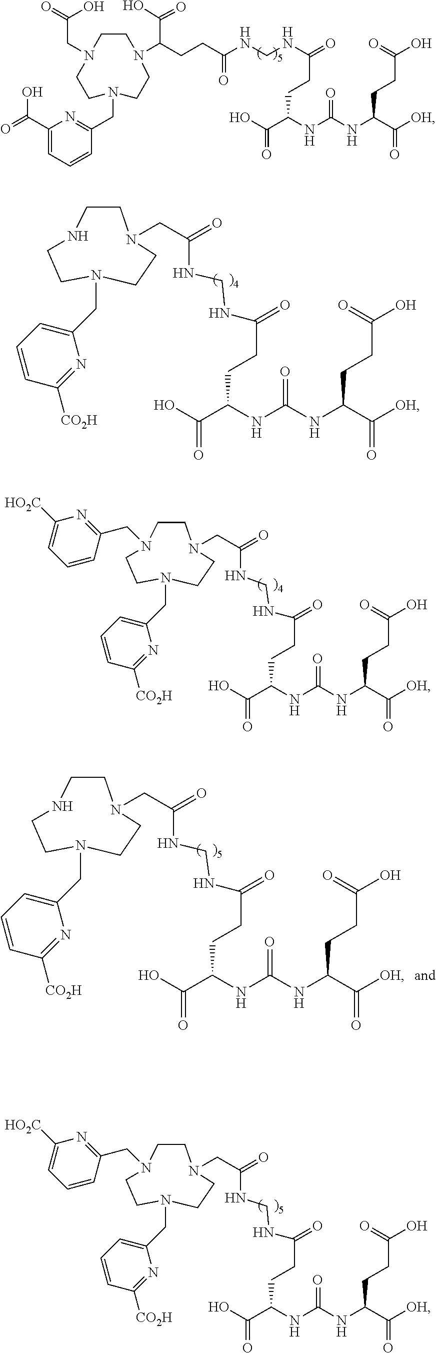
or a pharmaceutically acceptable salt of the compound.

2. The compound of claim 1, wherein
a) Y₃ is Z₁-L(A), Z₁-L(A)(B), L(A) or L(A)(B) and Y₄ is —H;
b) Y₄ is Z₁-L(A) or Z₁-L(A)(B) and Y₃ is —H; or
c) Y₁ and Y₂ are each, independently, alkyl-CO₂H, alkylaryl-CO₂H, alkylheteroaryl-CO₂H, alkylheteroaryl-(NO₂)CO₂H, alkyl-P(O)(OH)₂, alkylaryl-P(O)(OH)₂, alkylheteroaryl-P(O)(OH)₂ or alkylheteroaryl-(NO₂) P(O)(OH)₂, Y₃ is Z₁-L(A) or Z₁-L(A)(B) and Y₄ is —H, or Y₄ is Z₁-L(A) or Z₁-L(A)(B) and Y₃ is —H.

3. The compound of claim 2 having the structure:

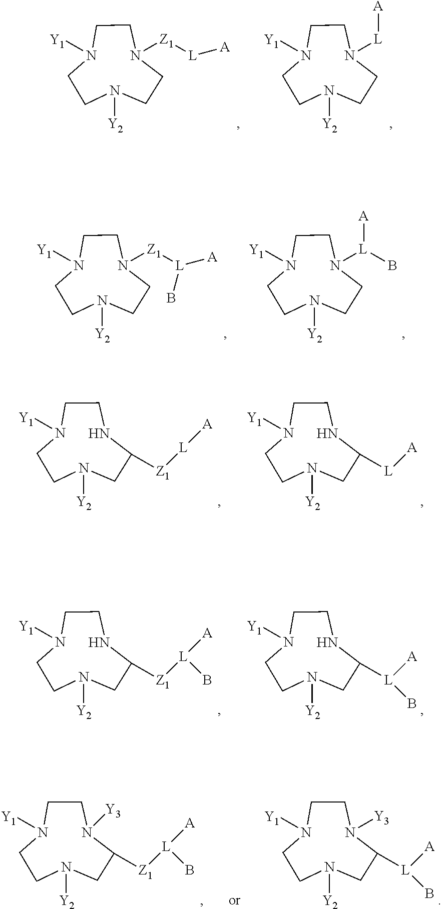

4. The compound of claim 1 having the structure: 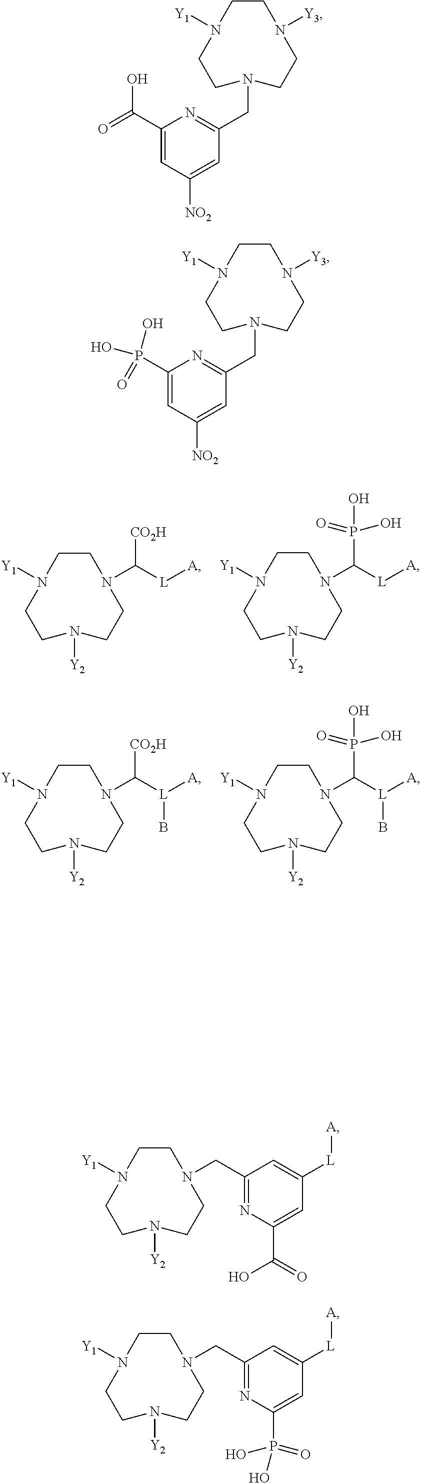 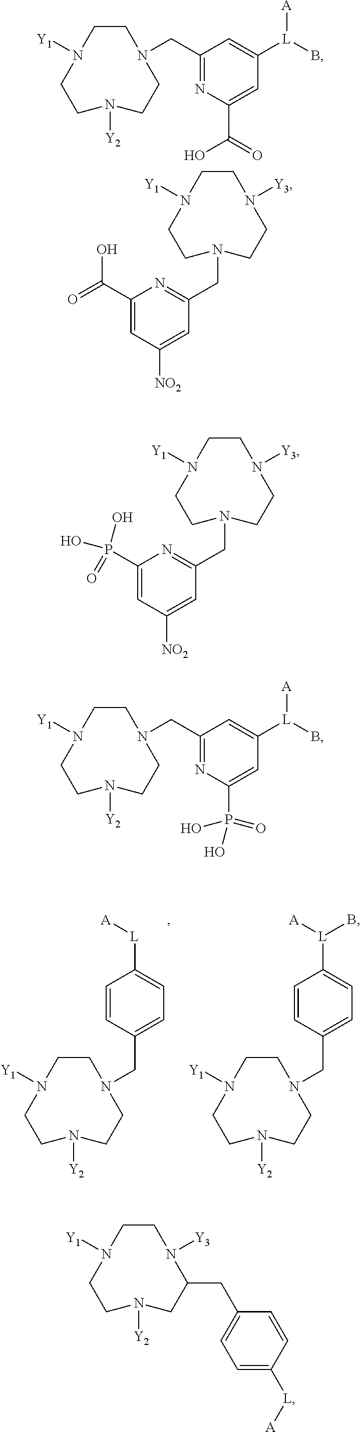 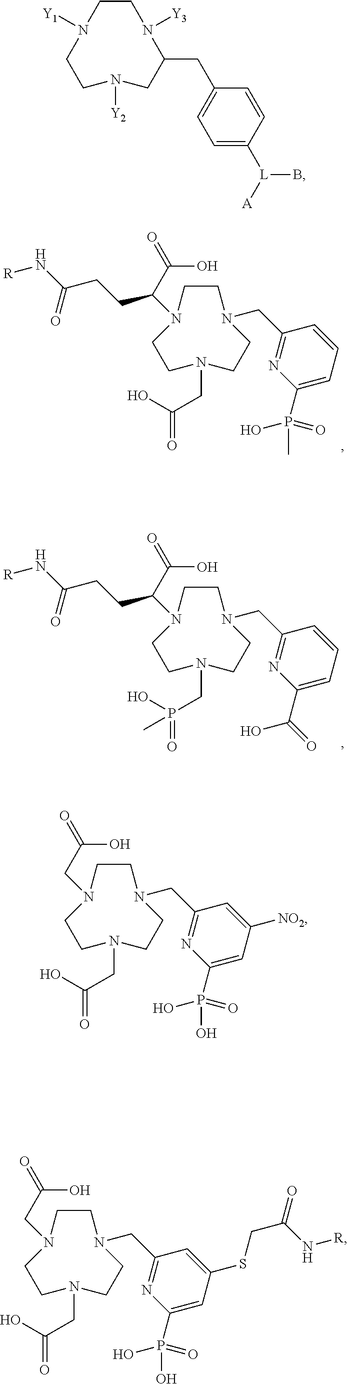 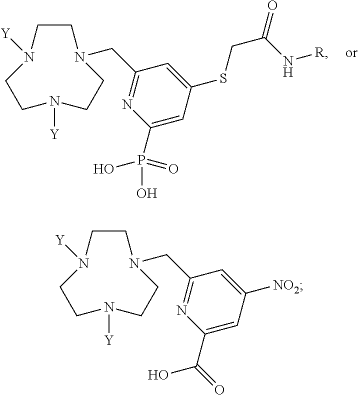
wherein R is H, alkyl, alkenyl, alkynyl, alkyl-aryl, alkyl-heteroaryl, aryl, heteroaryl, alkyl-CF₃ or —Si(alkyl)₃.

5. (canceled)

6. The compound of claim 4, wherein the targeting moiety A is
a. a moiety with specificity for a target protein on the surface of a cell;
b. a moiety with specificity for a target antigen on the surface of a cell; or
c. ((5-(2-(4-(aminomethyl)cyclohexane-1-carboxamido)-3-(naphthalen-2-yl)propanamido)-1-carboxypentyl)carbamoyl)glutamic acid or a derivative or fragment thereof; 2-[3-(1,3-dicarboxypropyl)ureido]pentanedioic acid (DUPA) or a derivative or fragment thereof; trastuzumab, bombesin or somatostatin or a derivative or fragment thereof; or
d. covalently attached to the chemical linker L or
wherein the albumin-binding moiety B is 4-(4-iodophenyl)butanoic acid or a derivative or fragment thereof, or the albumin-binding moiety B is 4-(4-methyl)butanoic acid or a derivative or fragment thereof;
wherein
a) the albumin-binding moiety B is covalently attached to the chemical linker L;
b) the targeting moiety A and the albumin-binding moiety B are both covalently attached to the chemical linker L; or the bond between the albumin-binding moiety B and the chemical linker L is formed by reacting a first terminal reactive group on the albumin-binding moiety B with a second terminal reactive group on the chemical linker L;
wherein L has the structure: 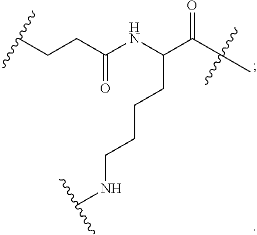 [SMILES: *CCC(=O)NC(CCCCN*)C(*)=O]
or wherein the chemical linker L is a releasable linker or a non-releasable linker.

7.-11. (canceled)

12. The compound of claim 1, wherein Y₁ and Y₂ are each, independently, —H, alkyl-CO₂H, alkylaryl-CO₂H, alkylheteroaryl-CO₂H, alkylheteroaryl-(NO₂)(CO₂H), alkyl-P(O)(OH)₂, alkylaryl-P(O)(OH)₂, alkylheteroaryl-P(O)(OH)₂ or alkylheteroaryl-(NO₂)(P(O)(OH)₂).

13. The compound of claim 12, wherein
a) one of Y¹ or Y₂ is —H, or each of Y₁ and Y₂ is —H;
b) one of Y¹ or Y₂ is alkyl-CO₂H, or each of Y₁ and Y₂ is alkyl-CO₂H;
c) one of Y¹ or Y₂ is alkylaryl-CO₂H or alkylheteroaryl-CO₂H, or each of Y₁ and Y₂ is alkylaryl-CO₂H or alkylheteroaryl-CO₂H;
d) one of Y¹ or Y₂ is alkyl-P(O)(OH)₂, or each of Y₁ and Y₂ is alkyl-P(O)(OH)₂;
e) one of Y¹ or Y₂ is alkylaryl-P(O)(OH)₂ or alkylheteroaryl-P(O)(OH)₂, or each of Y₁ and Y₂ is alkylaryl-P(O)(OH)₂ or alkylheteroaryl-P(O)(OH)₂;
f) Y₁ is alkyl-CO₂H, and Y₂ is alkylaryl-CO₂H or alkylheteroaryl-CO₂H;
g) Y₁ is alkyl-CO₂H, and Y₂ is alkylaryl-P(O)(OH)₂ or alkylheteroaryl-P(O)(OH)₂; or
h) Y₁ is alkyl-P(O)(OH)₂, and Y₂ is alkylaryl-CO₂H or alkylheteroaryl-CO₂H.

14. The compound of claim 1 having the structure:

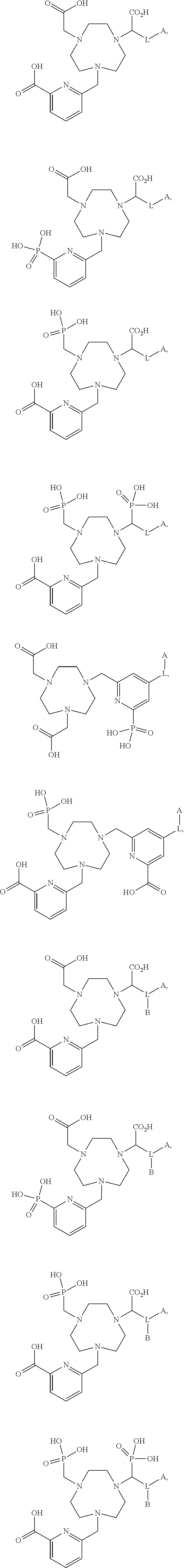

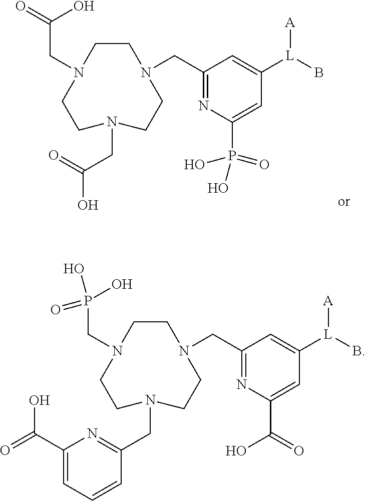

15. A metal complex comprising the compound of claim 1, wherein the compound coordinates to a metal or metal-ion (M), wherein the metal is Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³² La), Lanthanum-135 (¹³⁵ La), Yttrium-86 (⁸⁶Y), Yttrium-90 (⁹⁰Y), Lutetium-177 (¹⁷⁷Lu), Terbium-149 (¹⁴⁹Tb), Terbium-152 (¹⁵² Tb), Terbium-155 (¹⁵⁵Tb) or Terbium-161 (¹⁶¹ Tb).

16. The metal complex of claim 15 having the structure: 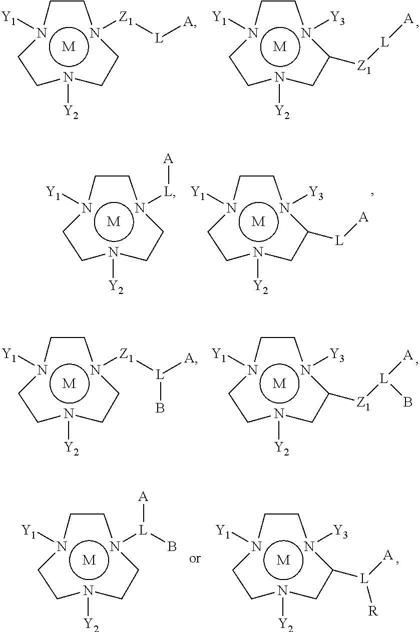
or a pharmaceutically acceptable salt thereof.

17. The metal complex of claim 16 having the structure:

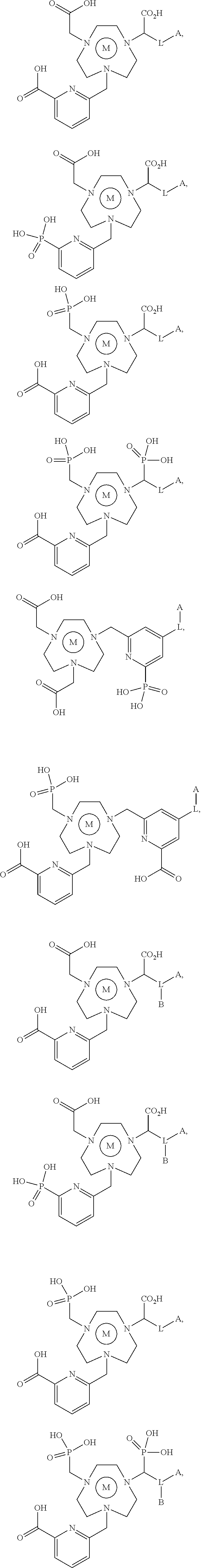

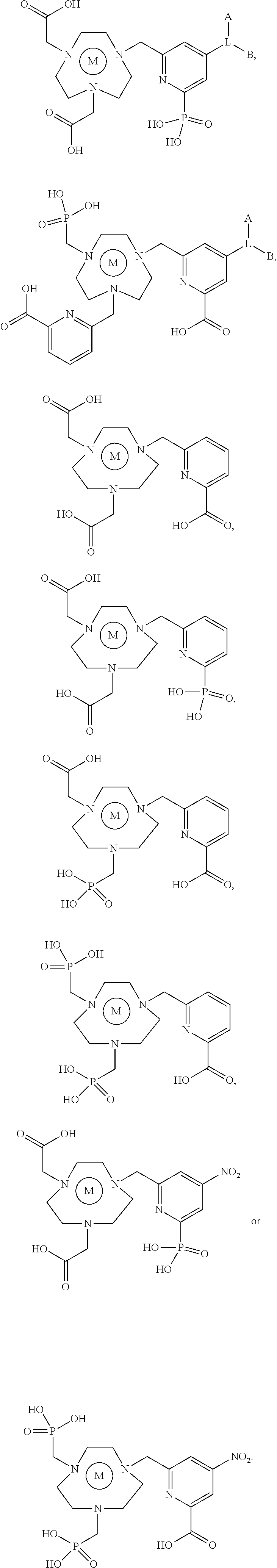

18. (canceled)

19. (canceled)

20. The compound of claim 17 having the structure: 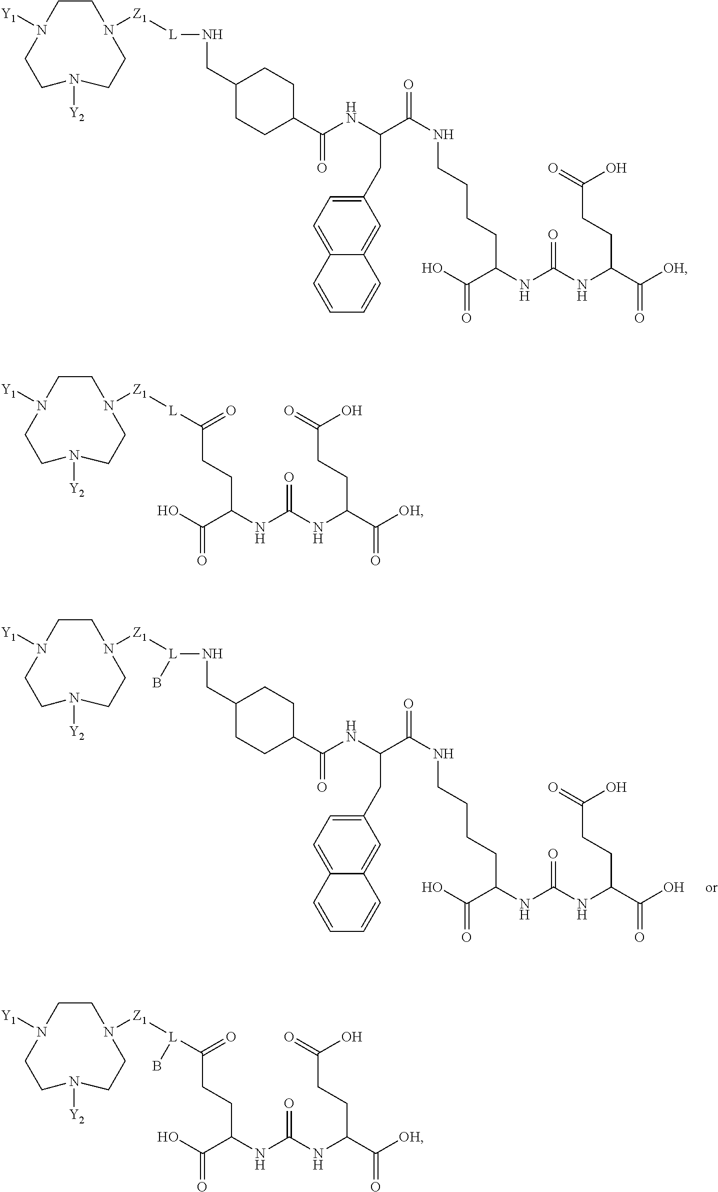
wherein B is an albumin-binding moiety.

21. The compound of claim 1,
a) the chemical linker L is alkyl, alkenyl, alkynyl, alkyl-O-alkyl, alkyl-O-alkyl-O-alkyl, alkyl-NH, alkyl-NH-alkyl, alkyl-C(O)O-alkyl, alkyl-OC(O)-alkyl alkyl-CO-alkyl, alkyl-C(O)NH-alkyl, alkyl-NHC(O)-alkyl or alkyl-C(O)NH-alkyl-NH or combinations thereof;
b) Y₁ and Y₂ are each, independently, —H, 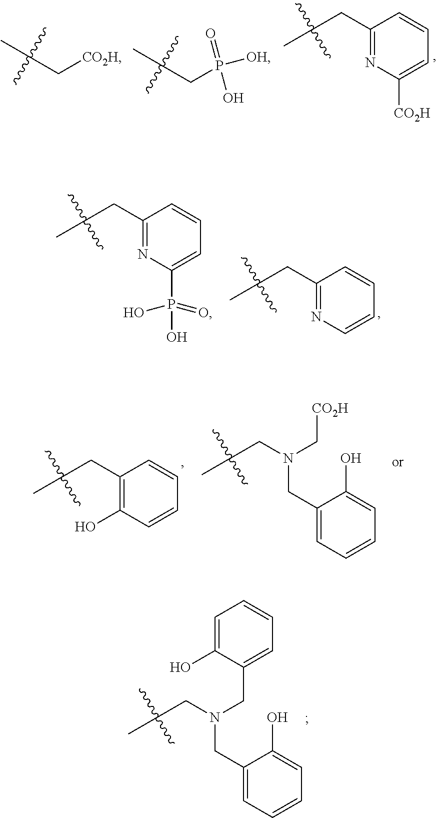
or
c) Z₁ is 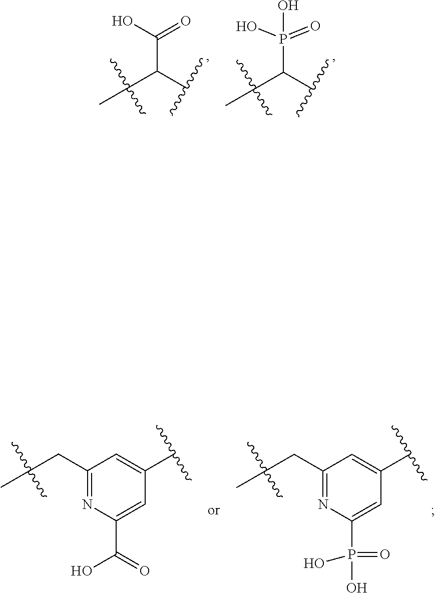 [SMILES: *C(*)C(=O)O ; *C(*)P(=O)(O)O ; *Cc1cc(*)cc(C(=O)O)n1 ; *Cc1cc(*)cc(P(=O)(O)O)n1]
or
the compound of claim 1 having the stucture: 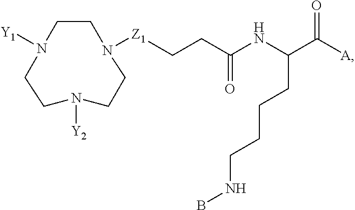
wherein
a) Z₁ is 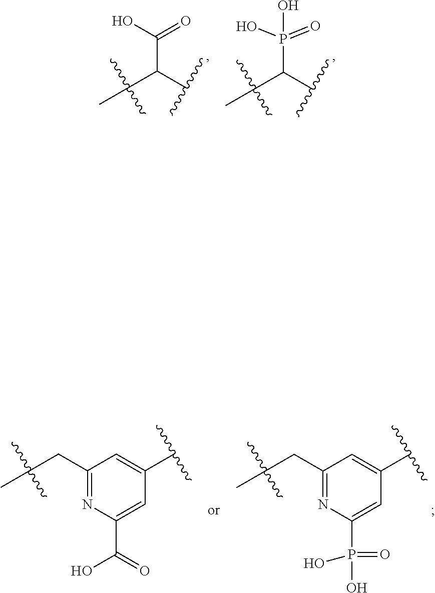 [SMILES: *C(*)C(=O)O ; *C(*)P(=O)(O)O ; *Cc1cc(*)cc(C(=O)O)n1 ; *Cc1cc(*)cc(P(=O)(O)O)n1]
b) Y₁ and Y₂ are each, independently, —H, 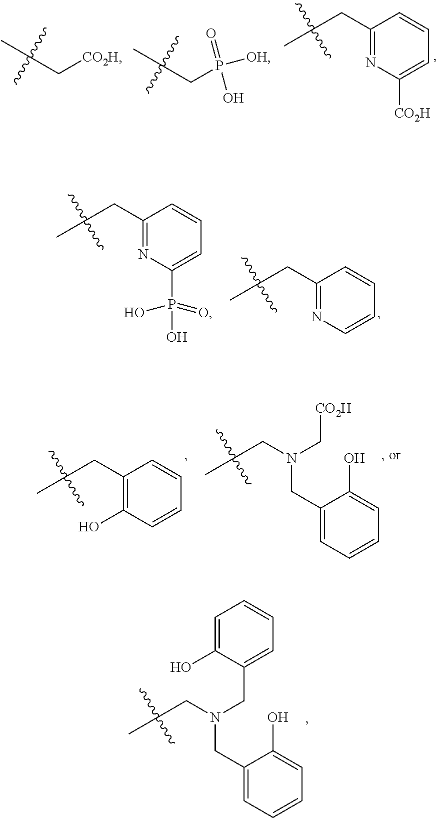
or
c) B has the structure: 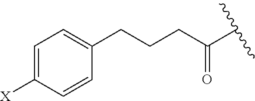
wherein X is halogen or alkyl.

22. The compound of claim 21 having the structure:

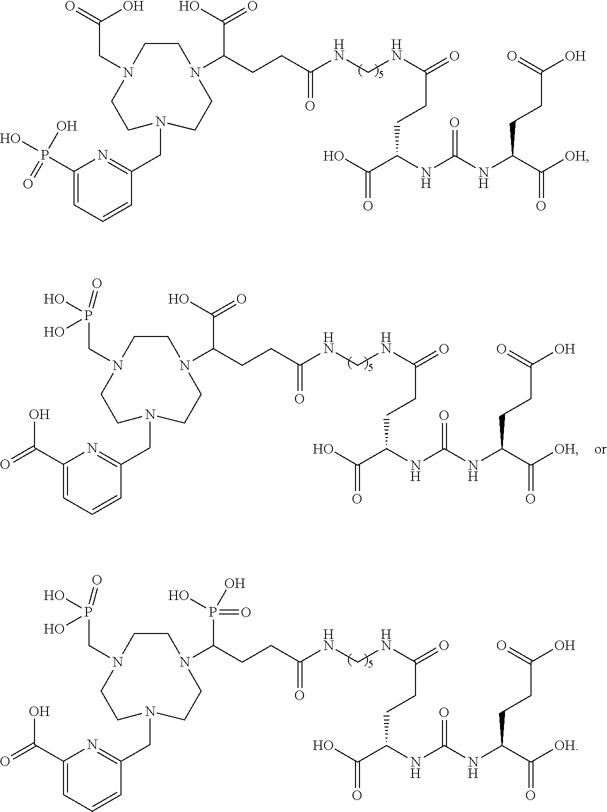

23.-26. (canceled)

27. The compound of claim 21 having the structure: 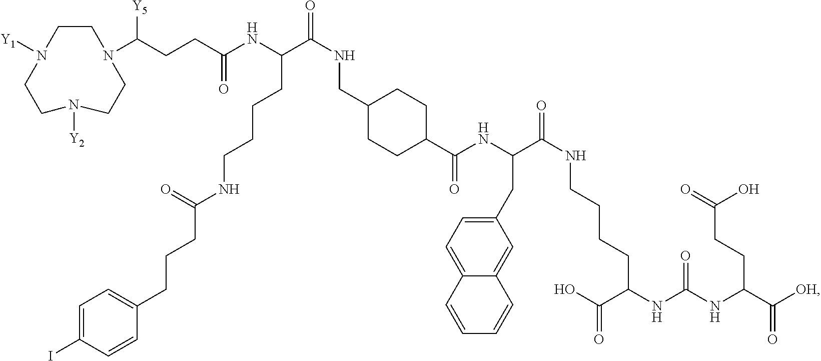
wherein
Y₁ and Y₂ are each, independently —H, 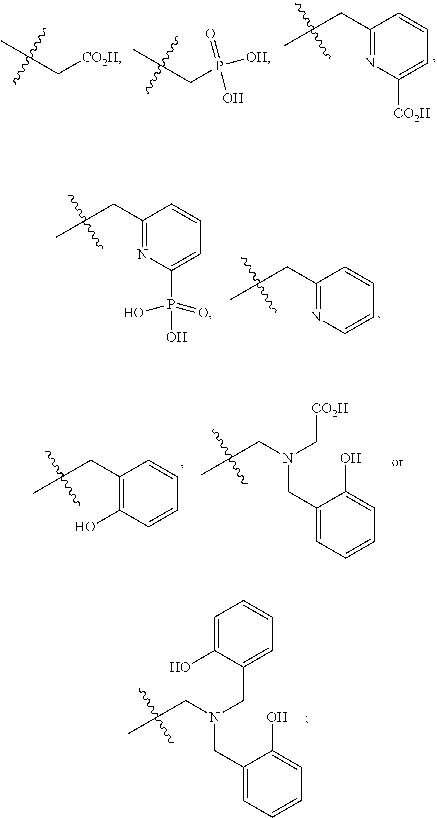
and
Y₅ is —CO₂H or —P(O)(OH)₂,
or a pharmaceutically acceptable salt of the compound.

28. (canceled)

29. (canceled)

30. The compound of claim 27, having the structure: 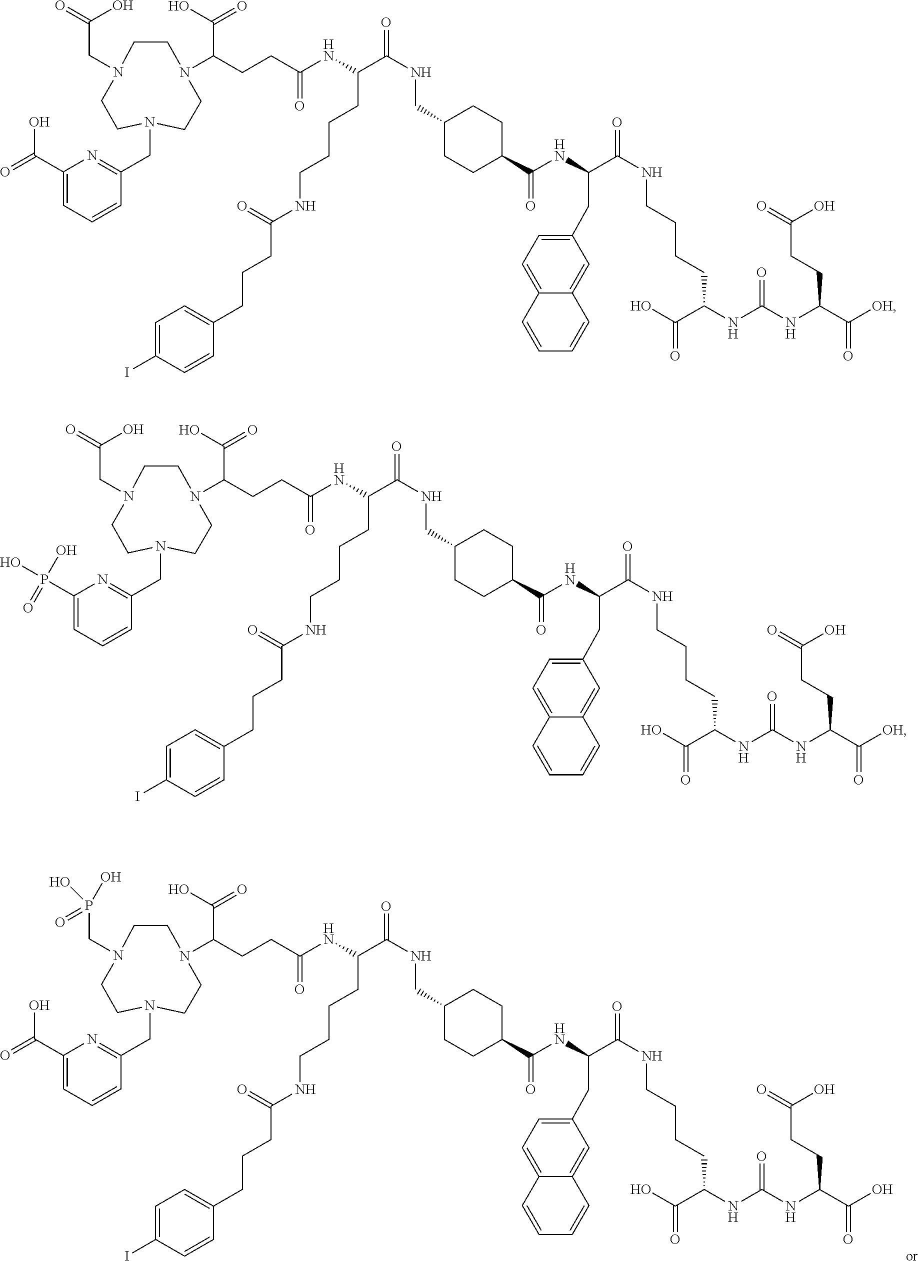 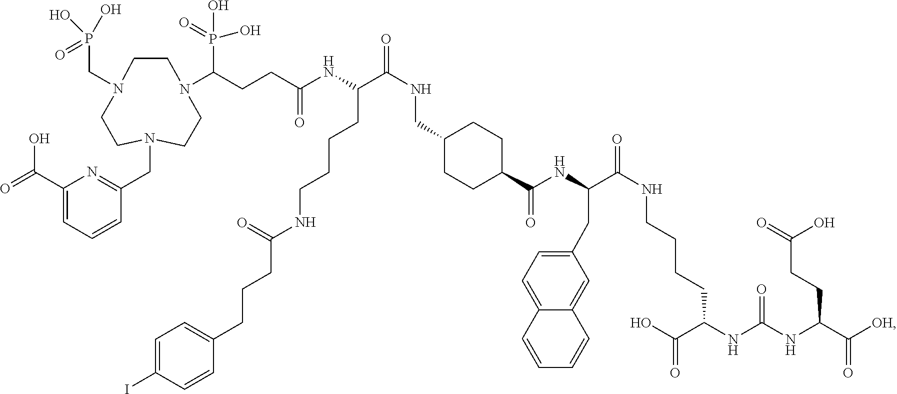
or a pharmaceutically acceptable salt of the compound.

31. (canceled)

32. The metal complex of claim 15 having the structure: 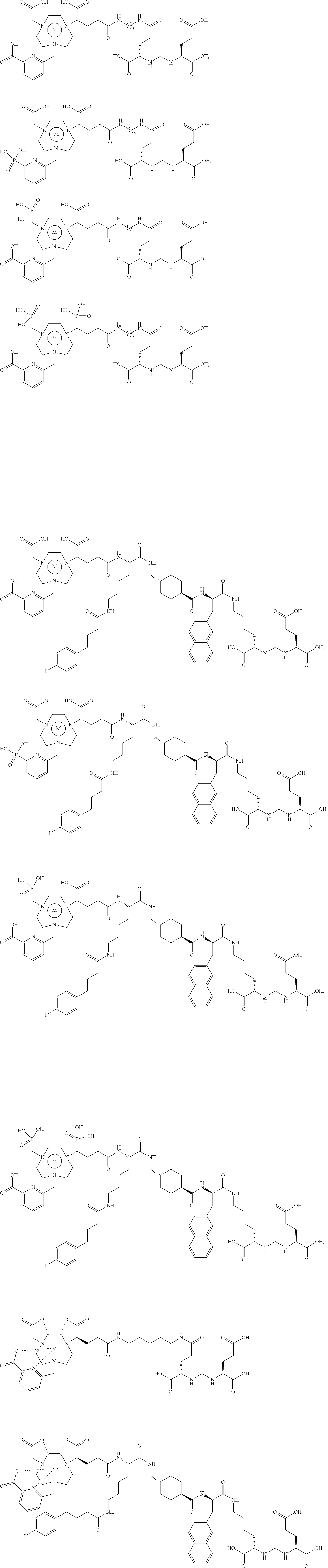 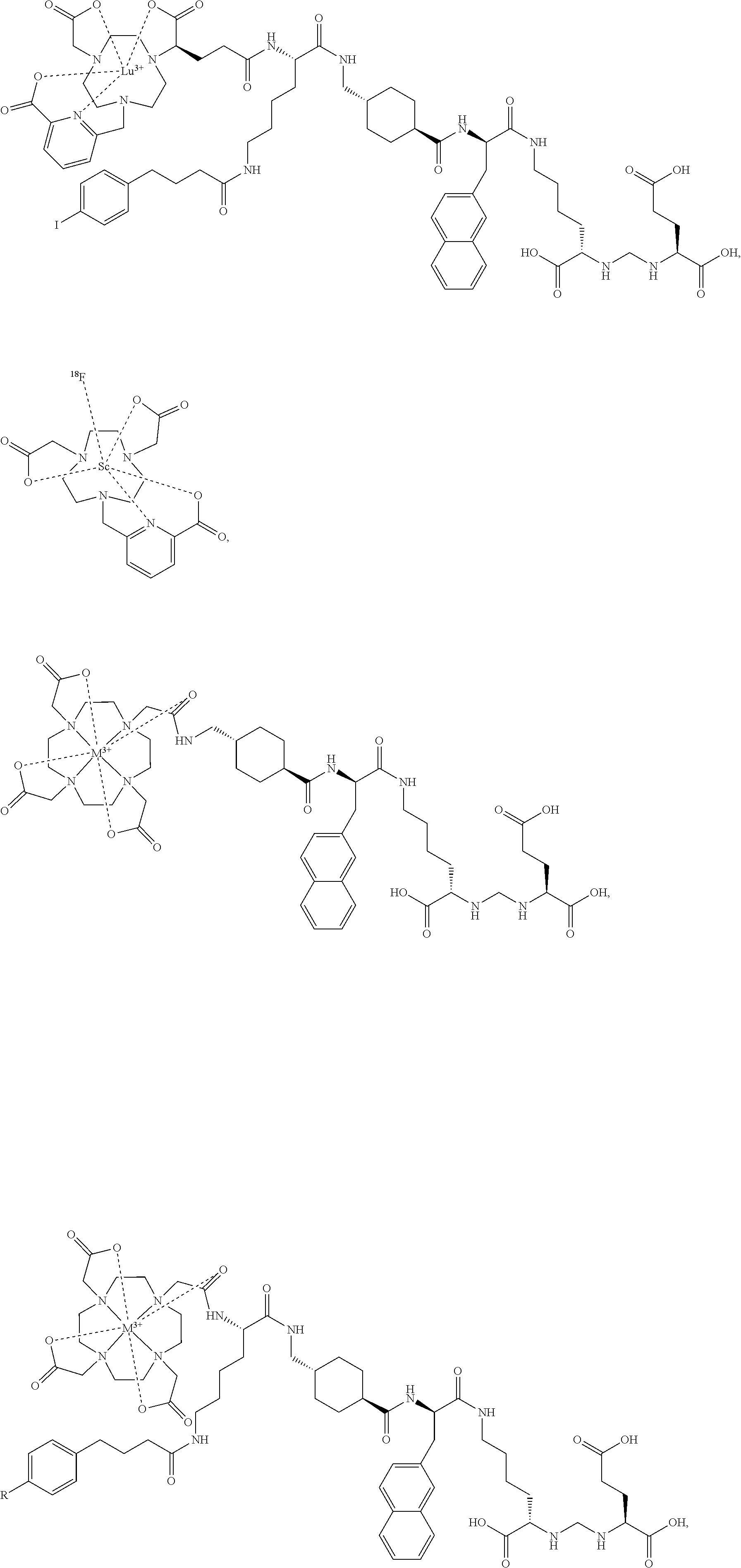 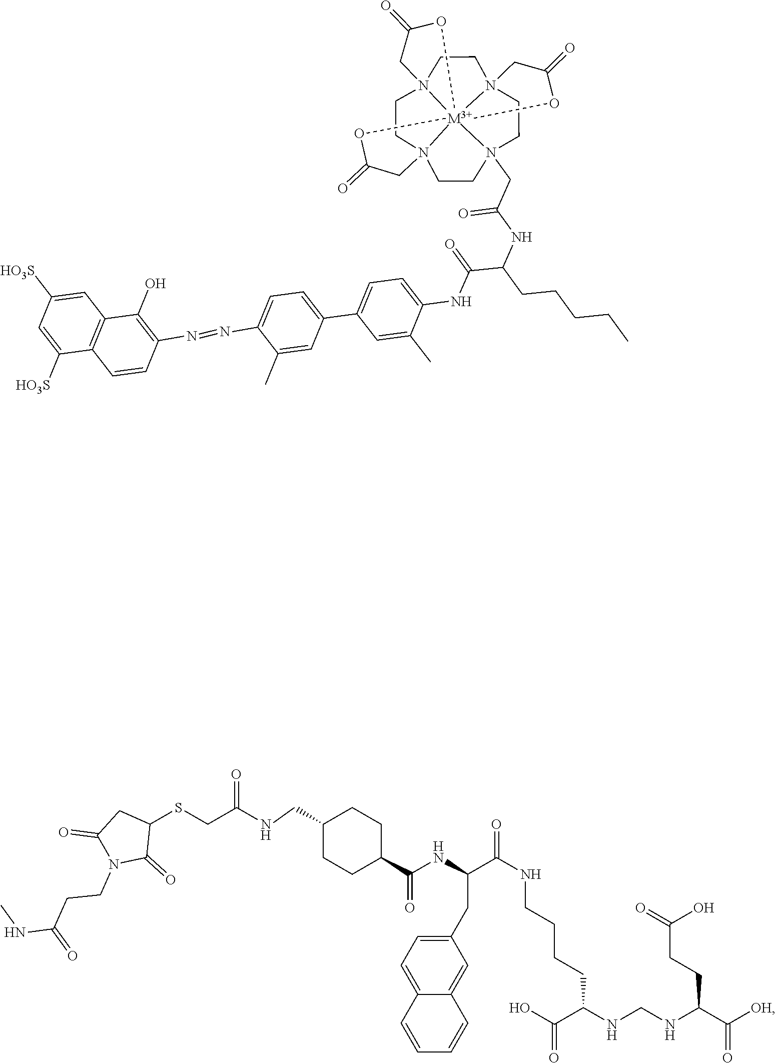 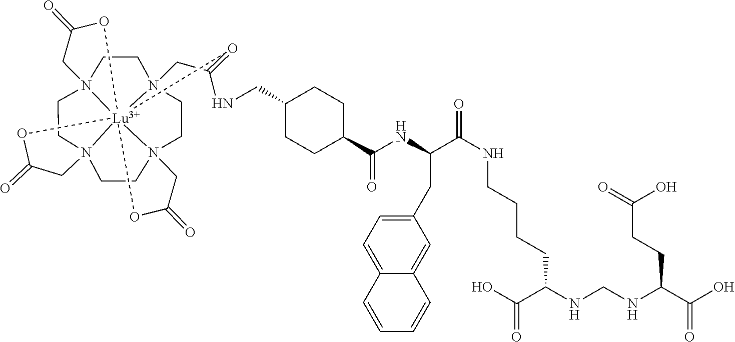
or a pharmaceutically acceptable salt of the compound.

33. A pharmaceutical composition comprising the metal complex of claim 15, and a pharmaceutically acceptable carrier.

34. A method of detecting target cells in a subject comprising administering an effective amount of the metal complex claim 15 to the subject, and imaging the subject with a molecular imaging device to detect the metal complex in the subject.

35. A method of imaging target cells in a subject comprising:
1) administering to the subject an effective amount of the metal complex of claim 15 or a pharmaceutically acceptable salt thereof, wherein the compound specifically accumulates at the target cells in the subject;
2) detecting in the subject the location of the metal complex or the composition; and
3) obtaining an image of the target cells in the subject based on the location of the metal complex in the subject.

36.-55. (canceled)
